(19) United States
(12) Patent Application Publication
(10) Pub. No.: US 2025/0221608 A1
(43) Pub. Date: Jul. 10, 2025

(54) ERGONOMIC URETEROSCOPE WITH POWER-ASSISTED CONTROLLED CANNULA ROTATION AND CONCURRENTLY OPERABLE CANNULA TIP DEFLECTION CONTROL

(71) Applicant: MicronVision Corp., Bellevue, WA (US)

(72) Inventor: Xiaolong OUYANG, Bellevue, WA (US)

(21) Appl. No.: 19/089,980

(22) Filed: Mar. 25, 2025

Related U.S. Application Data
(63) Continuation-in-part of application No. 18/628,133, filed on Apr. 5, 2024.
Application No. 18/628,133 is a continuation-in-part of application No. 18/083,209, filed on Dec. 16, 2022.
Application No. 18/083,209 is a continuation-in-part of application No. 17/941,884, filed on Sep. 9, 2022.
(63) Continuation-in-part of application No. 18/212,621, filed on Jun. 21, 2023.
Application No. 18/212,621 is a division of application No. 17/720,143, filed on Apr. 13, 2022, now Pat. No. 11,771,304.
Application No. 17/720,143 is a continuation-in-part of application No. 17/521,397, filed on Nov. 8, 2021, now Pat. No. 11,980,342.
(60) Provisional application No. 63/417,340, filed on Oct. 19, 2022.
(60) Provisional application No. 63/256,634, filed on Oct. 18, 2021.
(60) Provisional application No. 63/282,108, filed on Nov. 22, 2021.
(60) Provisional application No. 63/283,367, filed on Nov. 26, 2021.
(60) Provisional application No. 63/332,233, filed on Apr. 18, 2022.
(60) Provisional application No. 63/176,307, filed on Apr. 18, 2021.
(60) Provisional application No. 63/112,739, filed on Nov. 12, 2020.
(60) Provisional application No. 63/113,960, filed on Nov. 15, 2020.
(60) Provisional application No. 63/118,617, filed on Nov. 25, 2020.
(60) Provisional application No. 63/128,105, filed on Dec. 20, 2020.
(60) Provisional application No. 63/138,528, filed on Jan. 18, 2021.
(60) Provisional application No. 63/295,913, filed on Jan. 2, 2022.
(60) Provisional application No. 63/299,829, filed on Jan. 14, 2022.
(60) Provisional application No. 63/299,960, filed on Jan. 15, 2022.
(60) Provisional application No. 63/302,563, filed on Jan. 25, 2022.
(60) Provisional application No. 63/303,690, filed on Jan. 27, 2022.
(60) Provisional application No. 63/310,336, filed on Feb. 15, 2022.
(60) Provisional application No. 63/743,649, filed on Jan. 10, 2025.

(51) Int. Cl. A61B 1/00; A61B 1/015; A61B 1/018; A61B 1/05; A61B 17/02; A61B 17/34; G16H 30/40

(52) U.S. Cl. CPC A61B 1/00032; A61B 1/00131; A61B 1/015; A61B 1/018; A61B 1/05; A61B 17/0218; A61B 17/3421; G16H 30/40

(57) ABSTRACT

A ureteroscope or endoscope in which a reusable portion is inserted into a pistol-grip handle of a single-use portion and a resilient cap covers the reusable portion and a hollow end of the handle. A hub assembly and cannula of the single-use portion can be rotated either under power or manually, and a tip portion of the cannula can be deflected manually or under power in a deflection plane. One or more ports can be attached to the hub assembly communicate with one or more lumen in the cannula. The ports are aligned in a luer plane that may be in-line with the deflection plane or at an angle relative to the deflection plane. A remote display connects to the ureteroscope or endoscope wirelessly or through a cable. A display shows the current relative orientation of an image sensor and a working channel and the current direction of view of the image sensor relative to the patient.

### REFERENCE TO RELATED APPLICATIONS

This application is a continuation-in-part of U.S. patent application Ser. No. 18/628,133 filed Apr. 5, 2024, which is a continuation-in-part of U.S. patent application Ser. No. 18/083,209 filed Dec. 16, 2022, which is a continuation-in-part of U.S. patent application Ser. No. 17/941,884 filed Sep. 9, 2022, which claim priority to U.S. Provisional Appln. Ser. Nos. 63/417,340 filed Oct. 19, 2022; 63/256,634 filed Oct. 18, 2021; 63/282,108 filed Nov. 22, 2021; 63/283,367 filed Nov. 26, 2021; and 63/332,233 filed Apr. 18, 2022.

This application is a continuation-in-part of U.S. patent application Ser. No. 18/212,621 filed Jun. 21, 2023, which is a divisional of U.S. patent application Ser. No. 17/720,143 filed Apr. 13, 2022 (now U.S. Pat. No. 11,771,304), which is a continuation-in-part of U.S. patent application Ser. No. 17/521,397 filed Nov. 8, 2021, which claim priority to U.S. Provisional Appln. Ser. Nos. 63/176,307 filed Apr. 4, 2021; 63/112,739 filed Nov. 12, 2020; 63/113,960 filed Nov. 15, 2020; 63/118,617 filed Nov. 25, 2020; 63/128,105 filed Dec. 20, 2020; 63/138,528 filed Jan. 18, 2021; 63/295,913 filed Jan. 2, 2022; 63/299,829 filed Jan. 14, 2022; 63/299,960 filed Jan. 15, 2022; 63/302,563 filed Jan. 25, 2022; 63/303,690 filed Jan. 27, 2022; and 63/310,336 filed Feb. 15, 2022.

This application also claims priority to U.S. Provisional Appln. Ser. No. 63/743,649 filed Jan. 10, 2025.

This application incorporates by reference the entirety of the foregoing non-provisional and provisional patent applications and claims the benefit of the filing date of each as well as of the applications that they incorporated by reference, directly or indirectly, and the benefit of which they claim, including U.S. provisional applications, U.S. non-provisional applications, and international applications.

This patent application incorporates by reference each of the following U.S. patents and U.S. and international (PCT) patent applications:
Ser. No. 16/972,989 filed Dec. 7, 2020;
PCT/US21/50095 filed Sep. 13, 2021;
PCT/US2017/053171 filed Sep. 25, 2017
Ser. No. 17/835,624 filed Jun. 8, 2022;
Ser. No. 16/363,209, filed Sep. 25, 2017, now U.S. Pat. No. 11,832,797;
Ser. No. 17/843,217, filed Jun. 17, 2022
PCT/US16/18670 filed Feb. 19, 2016;
Ser. No. 14/913,867 filed Feb. 23, 2016, now U.S. Pat. No. 10,874,287;
PCT/US16/65396 filed Dec. 7, 2016;
Ser. No. 15/371,858 filed Feb. 20, 2018, now U.S. Pat. No. 9,895,048;
Ser. No. 15/462,331 filed Mar. 17, 2017, now U.S. Pat. No. 10,524,636;
Ser. No. 15/651,526 filed Jul. 17, 2017, now U.S. Pat. No. 10,278,563;
Ser. No. 15/855,532 filed Dec. 27, 2017, now U.S. Pat. No. 10,292,571;
PCT/US18/14880 filed Jan. 23, 2018;
Ser. No. 16/407,028 filed May 8, 2019, now U.S. Pat. No. 11,253,141;
Ser. No. 16/413,160 filed May 15, 2019, now U.S. Pat. No. 10,869,592;
Ser. No. 16/447,251 filed Jun. 20, 2019, now U.S. Pat. No. 11,013,141;
PCT/US20/38349 filed Jun. 18, 2020;
PCT/US20/46018 filed Aug. 12, 2020;
Ser. No. 17/122,282 filed Dec. 15, 2020;
Ser. No. 17/145,466 filed Jan. 11, 2021, now U.S. Pat. No. 11,395,579;
Ser. No. 17/370,575 filed Jul. 8, 2021;
Ser. No. 17/349,674 filed Jun. 16, 2021;
Ser. No. 17/573,095 filed Jan. 24, 2022;
PCT/US2017/053171 filed Sep. 25, 2017
Ser. No. 17/835,624 filed Jun. 8, 2022;
Ser. No. 16/363,209 filed Sep. 25, 2017, now U.S. Pat. No. 11,832,797;
Ser. No. 17/843,217 filed Jun. 17, 2022;
Ser. No. 17/362,043 filed Jun. 29, 2021, now U.S. Pat. No. 11,350,816;
Ser. No. 17/473,587 filed Sep. 13, 2021, now U.S. Pat. No. 11,330,973;
Ser. No. 17/745,526 filed May 16, 2022;
Ser. No. 17/521,397 filed Nov. 8, 2021;
Ser. No. 17/720,143 filed Apr. 13, 2022;
Ser. No. 18/212,621 filed Jun. 21, 2023;
Ser. No. 17/941,884 filed Sep. 9, 2022;
Ser. No. 17/835,624 filed Jun. 8, 2022, now U.S. Pat. No. 11,684,248
Ser. No. 18/083,209 filed Dec. 16, 2022;
Ser. No. 18/113,395 filed Feb. 23, 2023;
Ser. No. 18/211,486 filed Jun. 19, 2023;
Ser. No. 18/233,282 filed Aug. 11, 2023;
Ser. No. 18/374,740 filed Sep. 29, 2023;
Ser. No. 14/913,867 filed Feb. 23, 2016, now U.S. Pat. No. 10,874,287;
Ser. No. 16/407,028 filed May 18, 2019, now U.S. Pat. No. 11,253,141;
Ser. No. 16/413,160 filed May 15, 2019, now U.S. Pat. No. 10,869,592;
Ser. No. 17/122,282 filed Dec. 15, 2020, now U.S. Pat. No. 11,844,498;
Ser. No. 15/371,858 filed Dec. 7, 2016, now U.S. Pat. No. 9,895,048;
Ser. No. 13/094,415 filed Apr. 26, 2011, now U.S. Pat. No. 9,649,014;
Ser. No. 15/462,331 filed Mar. 17, 2017, now U.S. Pat. No. 10,524,636;
Ser. No. 15/651,526 filed Jul. 17, 2017, now U.S. Pat. No. 10,278,563;
Ser. No. 15/855,532 filed Dec. 27, 2017, now U.S. Pat. No. 10,292,571;
Ser. No. 15/484,953 filed Apr. 11, 2017, now U.S. Pat. No. 10,426,320;
PCT/US2020/038349 filed Jun. 18, 2020, now U.S. Pat. No. 11,013,396;
Ser. No. 17/370,575 filed Jul. 8, 2021;
Ser. No. 18/209,947 filed Jun. 14, 2023;
Ser. No. 17/145,466 filed Jan. 11, 2021, now U.S. Pat. No. 11,395,579;
Ser. No. 16/664,082 filed Oct. 25, 2019, now U.S. Pat. No. 11,071,442;
Ser. No. 17/349,674 filed Jun. 16, 2021
PCT/US2020/046018 filed Aug. 12, 2020;
Ser. No. 17/583,095 filed Jan. 24, 2022;
Ser. No. 13/276,839 filed Oct. 19, 2011, now U.S. Pat. No. 8,702,594; and
Ser. No. 13/094,415 filed Apr. 26, 2011, now U.S. Pat. No. 9,649,014.

### FIELD

This patent specification relates to endoscopes and more specifically to endoscopes and ureteroscopes that facilitate medical procedures, have lower manufacturing cost, and can be partly disposable and partly reusable, or fully disposable.

### DESCRIPTION OF RELATED ART

Clinicians use ureteroscopes to examine the upper urinary tract. This typically involves passing a ureteroscope cannula through the urethra into the bladder and then from the bladder into the ureter and possibly into a kidney. The procedure is useful in the diagnosis and treatment of disorders such as kidney stones and urothelial carcinoma of the upper urinary tract. Smaller stones in the bladder or lower ureter can be removed in one piece, while bigger ones are usually broken before removal during ureteroscopy. The examination may be performed with a flexible, semi-rigid or rigid device while the patient typically is under anesthesia. In pyeloscopy, the ureteroscope is designed to reach all the way to the renal pelvis, thereby allowing visualization of the entire drainage system of the kidney. The ureteroscope can contain an instrument port which allows for introduction of laser fibers to fragment stones, and micro-baskets to retrieve stone fragments. Ureteroscopes are a form of endoscopes that are especially useful or adapted for ureteroscopy and in some cases pyeloscopy as well. One example is a ureteroscope system offered by Boston Scientific—see https://www.bostonscientific.com/content/dam/bostonscientific/uro/lithovue-elite/URO-1328704-AA-Litho Vue_Elite_Brochure_US_510k-cleared_DIGITAL.pdf. Known commercially available ureteroscopes and their supporting equipment are believed to be expensive to manufacture and challenging to use, in part because they are not particularly ergonomic to operate during a medical procedure. For example, some may require unnatural and tiring ways of holding the ureteroscope during a medical procedure, excessive hand movement and wrist and elbow rotation to guide the distal tip of the ureteroscope to a target region in the patient and may lack convenient and unambiguous indications of where is the distal tip of the ureteroscope relative to a target region and how it is oriented relative to the target region.

Conventional endoscopy or direct vision used to examine the interior of a hollow organ or cavity of the body, uses a complex lens system for transmitting the image from the distal tip of the endoscope to a viewer. The lens system is typically an objective lens plus a relay lens system in the case of rigid endoscopes or a bundle of optic fibers in the case of flexible endoscopes. In the case of both rigid and flexible conventional endoscopes, the lens or fiber optic system is relatively expensive and is intended to be re-used many times. Therefore, stringent decontamination and disinfection procedures need to be carried out after each use.

Disposable endoscopy is a more recent category of endoscopic instruments. In some cases, the manufacture of endoscopes can be made sufficiently inexpensive to be used on a single patient only. Disposable or single-use endoscopy lessens the risk of cross-contamination and hospital acquired diseases, making it possible to perform procedures in doctor offices as well as in clinics and hospitals, and reduce the overall cost of medical procedures as they avoid expenses associated with sterilizing and maintaining traditional endoscopes and personnel needed for such maintenance.

The subject matter described or claimed in this patent specification is not limited to embodiments that solve any specific disadvantages or that operate only in environments such as those described above. Rather, the above background is only provided to illustrate one exemplary technology area where some embodiments described herein may be practiced.

### BRIEF DESCRIPTION OF THE DRAWINGS

To further clarify the above and other advantages and features of the subject matter of this patent specification, specific examples of embodiments thereof are illustrated in the appended drawings. It should be appreciated that these drawings depict only illustrative embodiments and are therefore not to be considered limiting of the scope of this patent specification or the appended claims, and that components of one or more of the embodiments may be used in any other embodiments as needed or suitable. The term endoscope as used in this patent specification is intended to encompass a ureteroscope. The subject matter hereof will be described and explained with additional specificity and detail through the accompanying drawings in which:

FIG. 1 is a perspective view of a first side of an exemplary embodiment of an endoscope according to the present disclosure, illustrating a single-use portion that includes an exemplary embodiment of a powered rotation drive system within a housing assembly, a hub assembly operatively coupled to the housing assembly, a cannula operatively coupled to the hub assembly, an imaging module at a distal part of the cannula and a cap in a closed position enclosing a wireless reusable portion inserted into an open bottom end of a handle of the housing assembly;

FIG. 2 is a perspective view of a second side of the assembled endoscope of FIG. 1, illustrating the cap in an open position and an exemplary embodiment of reusable portion of the endoscope according to the present disclosure staged for insertion into the open bottom end of the handle;

FIG. 3 is side elevation view of the first side of the endoscope of FIG. 1, illustrating a port plane of ports of the hub assembly in-line with a deflection plane of the cannula, and illustrating a body of the hub assembly having indicia indicating a position of a lumen of the cannula;

FIG. 4 is a top plan view of the endoscope of FIG. 1, illustrating an exemplary embodiment of an image control switch used to adjust an image generated by the imaging module based upon an orientation of a camera relative to a target area within a patient's body;

FIG. 5 is an exploded perspective view of the endoscope of FIG. 1, illustrating the housing assembly having a hollow housing and hollow handle extending from the housing forming a pistol-grip type handle, and illustrating a motor of the powered rotation drive system;

FIG. 6 is an exploded perspective view of an exemplary embodiment of the reusable portion of the endoscope according to the present disclosure;

FIG. 7 is a perspective view of a first side of another exemplary embodiment of an endoscope according to the present disclosure, illustrating a single-use portion that includes an exemplary embodiment of a manual rotation drive system within a housing assembly, a hub assembly operatively coupled to the housing assembly, a cannula operatively coupled to the hub assembly, an imaging module at a distal part of the cannula and a cap in a closed position enclosing a wireless reusable portion inserted into an open bottom end of a handle of the housing assembly;

FIG. 8 is side elevation view of the first side of the endoscope of FIG. 7, illustrating a port plane of ports of the hub assembly in-line with a deflection plane of the cannula;

FIG. 9 is an exploded perspective view of the endoscope of FIG. 7, illustrating the housing assembly having a hollow housing and hollow handle extending from the housing forming a pistol-grip type handle, and illustrating a thumbwheel of the manual rotation drive system;

FIG. 10 is a perspective view of the endoscope of FIG. 7, illustrating the cap in an open position and an exemplary embodiment of reusable portion of the endoscope according to the present disclosure staged for insertion into the open bottom end of a handle of the housing assembly;

FIG. 11 is a perspective view of an external processing/display unit capable of interacting with the embodiments of an endoscope according to the present disclosure;

FIG. 12 is a side perspective view of a first side of another exemplary embodiment of an endoscope according to the present disclosure, illustrating a single-use portion that includes an exemplary embodiment of a powered rotation drive system within a housing assembly, a hub assembly operatively coupled to the housing assembly, a cannula operatively coupled to the hub assembly, an imaging module at a distal part of the cannula and a cap in a closed position enclosing a wired reusable portion inserted into an open bottom end of a handle of the housing assembly;

FIG. 13 is a perspective view of the first side of the assembled endoscope of FIG. 12, illustrating the cap in an open position and the wired reusable portion staged for insertion into the open bottom end of the handle;

FIG. 14 is a perspective view of an external processing/display unit capable of interacting with the embodiments of an endoscope according to the present disclosure;

FIG. 15 is a perspective view of a first side of another exemplary embodiment of an endoscope according to the present disclosure, illustrating a single-use portion that includes an exemplary embodiment of a powered rotation drive system within a housing assembly, a hub assembly operatively coupled to the housing assembly, a cannula operatively coupled to the hub assembly, an imaging module at a distal part of the cannula and a cap in a closed position enclosing a wireless reusable portion inserted into an open bottom end of a handle of the housing assembly;

FIG. 16 is side elevation view of the first side of the endoscope of FIG. 15, illustrating a second portion of the housing assembly removed revealing a port at a proximal part of a body of the housing assembly and a portion of a deflection control system;

FIG. 17 is a top plan view of the endoscope of FIG. 15, illustrating an exemplary embodiment of an image control switch used to adjust an image generated by the imaging module;

FIG. 18 is an exploded perspective view of the endoscope of FIG. 15, illustrating the housing assembly having a hollow body and hollow handle extending from the housing forming a pistol-grip type handle, and illustrating a motor of the powered rotation drive system;

FIG. 19 is a perspective view of a first side of another exemplary embodiment of an endoscope according to the present disclosure, illustrating a single-use portion that includes an exemplary embodiment of a powered rotation drive system within a housing assembly, a hub assembly operatively coupled to the housing assembly, a cannula operatively coupled to the hub assembly, an imaging module at a distal part of the cannula and a cap in a closed position enclosing a wired reusable portion inserted into an open bottom end of a handle of the housing assembly;

FIG. 20 is an exploded perspective view of the endoscope of FIG. 19, illustrating the housing assembly having a hollow body and hollow handle extending from the body forming a pistol-grip type handle, and illustrating a motor of the powered rotation drive system;

FIG. 21 is a top perspective view of a first side of another exemplary embodiment of an endoscope according to the present disclosure, illustrating a single-use portion that includes a housing assembly with a port extending therefrom, a hub assembly operatively coupled to the housing assembly, a cannula operatively coupled to the hub assembly, an imaging module at a distal part of the cannula, and a cap in a closed position enclosing a wireless reusable portion inserted into an open bottom end of a handle of the housing assembly;

FIG. 22 is side elevation view of the first side of the endoscope of FIG. 21, illustrating a sight member extending from the hub assembly and aligned with a deflection plane of the cannula;

FIG. 23 is a top plan view of the endoscope of FIG. 21, illustrating an exemplary embodiment of the port extending from a top of the housing assembly;

FIG. 24 is an exploded perspective view of the endoscope of FIG. 21, illustrating the housing assembly having a hollow body and hollow handle extending from the body forming a pistol-grip type handle;

FIG. 25 is a top perspective view of a first side of another exemplary embodiment of an endoscope according to the present disclosure, illustrating a single-use portion that includes a housing assembly with a port extending therefrom, a hub assembly operatively coupled to the housing assembly, a cannula operatively coupled to the hub assembly, an imaging module at a distal part of the cannula, and a cap in a closed position enclosing a wired reusable portion inserted into an open bottom end of a handle of the housing assembly;

FIG. 26 is an exploded perspective view of the endoscope of FIG. 25, illustrating the housing assembly having a hollow body and hollow handle extending from the body forming a pistol-grip type handle;

FIG. 27 is a side perspective view of a first side of another exemplary embodiment of an endoscope according to the present disclosure, illustrating a single-use portion that includes a housing assembly with a pair of ports extending therefrom, a hub assembly operatively coupled to the housing assembly, a cannula operatively coupled to the hub assembly, an imaging module at a distal part of the cannula, and a cap in a closed position enclosing a wireless reusable portion inserted into an open bottom end of a handle of the housing assembly;

FIG. 28 is a top perspective view of the first side of the endoscope of FIG. 27;

FIG. 29 is side elevation view of the first side of the endoscope of FIG. 27, illustrating a sight member extending from the hub assembly and aligned with a deflection plane of the cannula, and a rocker switch for activating a motor of the rotation drive system;

FIG. 30 is side elevation view of the second side of the endoscope of FIG. 27, illustrating a sight member extending from the hub assembly and aligned with a deflection plane of the cannula, and a rocker switch for activating a motor of the rotation drive system;

FIG. 31 is a top plan view of the endoscope of FIG. 27, illustrating an exemplary embodiment of the pair of ports extending from a top of the housing assembly;

FIG. 32 is a bottom plan view of the endoscope of FIG. 27;

FIG. 33 is an elevation view form the proximal part of the endoscope of FIG. 27;

FIG. 34 is an elevation view form the distal part of the endoscope of FIG. 27;

FIG. 35 is a side elevation view of the of the endoscope of FIG. 29 with the right housing cover of the housing assembly removed revealing the reusable portion of the endoscope, the deflection control system and the pair of ports extending from a top of the housing assembly;

FIG. 36 is an exploded perspective view of the endoscope of FIG. 27, illustrating the housing assembly having a hollow body and hollow handle extending from the body forming a pistol-grip type handle;

FIG. 37 is a side perspective view of a first side of another exemplary embodiment of an endoscope according to the present disclosure, illustrating a single-use portion that includes a housing assembly with a pair of ports extending therefrom, a hub assembly operatively coupled to the housing assembly, a cannula operatively coupled to the hub assembly, an imaging module at a distal part of the cannula, and a cap in a closed position enclosing a wired reusable portion inserted into an open bottom end of a handle of the housing assembly;

FIG. 38 is an exploded perspective view of the endoscope of FIG. 37, illustrating the housing assembly having a hollow body and hollow handle extending from the body forming a pistol-grip type handle;

FIG. 39 is a perspective view of the endoscope of FIG. 1, illustrating the endoscope in a starting position prior to insertion into a patient, where the hub assembly is positioned so that a luer plane is aligned with a deflection plane;

FIG. 40 is a perspective view of the endoscope of FIG. 39, illustrating the endoscope rotated counter-clockwise 90 degrees from the starting position;

FIG. 41 is a perspective view of the endoscope of FIG. 39, illustrating the endoscope rotated clockwise 90 degrees from the starting position;

FIG. 42a is a schematic representation of the endoscope of FIG. 40 inserted into the right kidney of a patient;

FIG. 42b is a schematic representation of the endoscope of FIG. 41 inserted into the left kidney of a patient;

FIG. 43 is a perspective view of the endoscope of FIG. 1, illustrating the endoscope in a starting position prior to insertion into a patient, where the hub assembly is positioned so that the luer plane is orthogonal to the deflection plane;

FIG. 44 is a perspective view of the endoscope of FIG. 43, illustrating the endoscope rotated counter-clockwise 90 degrees from the starting position;

FIG. 45 is a perspective view of the endoscope of FIG. 43, illustrating the endoscope rotated clockwise 90 degrees from the starting position;

FIG. 46a is a schematic representation of the endoscope of FIG. 44 with the hub assembly positioned so that the deflection plane is aligned with the patient's right kidney and the display of the external processing/display unit displays the image of the patient's right kidney in an upright orientation and displays an icon of the tip of the cannula showing the real time orientation of the imaging module and cannula lumen;

FIG. 46b is a schematic representation of the endoscope of FIG. 45 with the hub assembly positioned so that the deflection plane is aligned with the patient's left kidney and the display of the external processing/display unit displays the image of the patient's left kidney in an upright orientation and displays an icon of the tip of the cannula showing the real time orientation of the imaging module and cannula lumen;

FIG. 47 is a schematic representation of exemplary angles of deflection of the tip portion of the cannula according to the present disclosure;

FIG. 48 is a front elevation view of an exemplary embodiment of the tip portion of the cannula according to the present disclosure, illustrating an exemplary embodiment of the imaging module according to the present disclosure and a channel extending through the cannula from a distal part to a proximal part;

FIG. 49 is a tip deflection and cannula axial rotation chart that illustrates the orientation of the imaging module at various angles of rotation of the cannula;

FIG. 50a is schematic representation of the endoscope of FIG. 44 inserted into the right kidney of a patient and illustrating the orientation of the imaging module;

FIG. 50b is a schematic representation of the endoscope of FIG. 45 inserted into the left kidney of a patient and illustrating the orientation of the imaging module;

FIG. 51 is a top perspective view of a first side of another exemplary embodiment of an endoscope according to the present disclosure, illustrating a single-use portion that includes a housing assembly with a port extending therefrom, a hub assembly operatively coupled to the housing assembly, a cannula operatively coupled to the hub assembly, an imaging module at a distal part of the cannula, and a cap in a closed position enclosing a wireless reusable portion inserted into an open bottom end of a handle of the housing assembly;

FIG. 52 is side elevation view of the first side of the endoscope of FIG. 51, illustrating a sight member extending from a top and bottom of the hub assembly and aligned with a deflection plane of the cannula;

FIG. 53 is a top plan view of the endoscope of FIG. 51, illustrating an exemplary embodiment of the port extending from the top of the housing assembly;

FIG. 54 is a side elevation view of the housing assembly of the endoscope of FIG. 52 with the right housing cover of the housing assembly removed revealing the reusable portion of the endoscope, the deflection control system and a pair of ports extending from a top of the housing assembly;

FIG. 55 is an exploded perspective view of the endoscope of FIG. 51, illustrating the housing assembly having a hollow body and hollow handle extending from the body forming a pistol-grip type handle;

FIG. 56 is an exploded perspective view of another exemplary embodiment of the reusable portion of the endoscope according to the present disclosure;

FIG. 57 is a top perspective view of a first side of another exemplary embodiment of an endoscope according to the present disclosure, illustrating a single-use portion that includes a housing assembly with a port extending therefrom, a hub assembly operatively coupled to the housing assembly, a cannula operatively coupled to the hub assembly, an imaging module at a distal part of the cannula, and a cap in a closed position enclosing a wireless reusable portion inserted into an open bottom end of a handle of the housing assembly;

FIG. 58 is a bottom perspective view of the endoscope of FIG. 57, illustrating a single-use portion that includes a housing assembly with a port extending from a top of the housing assembly and a port extending from a bottom of the housing assembly;

FIG. 59 is side elevation view of the first side of the endoscope of FIG. 57, illustrating the ports extending from the top and bottom of the housing assembly, and a sight member extending from a top and bottom of the hub assembly and aligned with a deflection plane of the cannula;

FIG. 60 is a top plan view of the endoscope of FIG. 59, illustrating the port extending from the top of the housing assembly;

FIG. 61 is a side elevation view of the housing assembly of the endoscope of FIG. 59 with the right housing cover of the housing assembly removed revealing the reusable portion of the endoscope, the deflection control system and the ports extending from the top and bottom of the housing assembly;

FIG. 62 is an exploded perspective view of the endoscope of FIG. 57, illustrating the housing assembly having a hollow body and hollow handle extending from the body forming a pistol-grip type handle;

FIG. 63 is a top perspective view of a first side of another exemplary embodiment of an endoscope according to the present disclosure, illustrating a single-use portion that includes a housing assembly with a port extending therefrom, a hub assembly operatively coupled to the housing assembly, a cannula operatively coupled to the hub assembly, an imaging module at a distal part of the cannula, and a cap in a closed position enclosing a wireless reusable portion inserted into an open bottom end of a handle of the housing assembly;

FIG. 64 is a bottom perspective view of the endoscope of FIG. 63, illustrating a single-use portion that includes a housing assembly with a port extending from a top of the housing assembly and a port extending from a bottom of the housing assembly;

FIG. 65 is side elevation view of the first side of the endoscope of FIG. 63, illustrating the ports extending from the top and bottom of the housing assembly, and a sight member extending from a top and bottom of the hub assembly and aligned with a deflection plane of the cannula;

FIG. 66 is a top plan view of the endoscope of FIG. 63, illustrating the port extending from the top of the housing assembly;

FIG. 67 is a side elevation view of the housing assembly of the endoscope of FIG. 65 with the right housing cover of the housing assembly removed revealing the reusable portion of the endoscope, the deflection control system and the ports extending from the top and bottom of the housing assembly;

FIG. 68 is an exploded perspective view of the endoscope of FIG. 63, illustrating the housing assembly having a hollow body and hollow handle extending from the body forming a pistol-grip type handle;

FIG. 69 is a top perspective view of a first side of another exemplary embodiment of an endoscope according to the present disclosure, illustrating a single-use portion that includes a housing assembly with a port extending therefrom, a hub assembly operatively coupled to the housing assembly, a cannula operatively coupled to the hub assembly, an imaging module at a distal part of the cannula, and a cap in a closed position enclosing a wireless reusable portion inserted into an open bottom end of a handle of the housing assembly;

FIG. 70 is a bottom perspective view of the endoscope of FIG. 69, illustrating a single-use portion that includes a housing assembly with a port extending from a top of the housing assembly and a port extending from a bottom of the housing assembly;

FIG. 71 is side elevation view of the first side of the endoscope of FIG. 69, illustrating the ports extending from the top and bottom of the housing assembly, and a sight member extending from a top and bottom of the hub assembly and aligned with a deflection plane of the cannula;

FIG. 72 is a top plan view of the endoscope of FIG. 69, illustrating the port extending from the top of the housing assembly;

FIG. 73 is a side elevation view of the housing assembly of the endoscope of FIG. 71 with the right housing cover of the housing assembly removed revealing the reusable portion of the endoscope, the deflection control system and the ports extending from the top and bottom of the housing assembly, and FIG. 73a is a perspective view of a detail of FIG. 73;

FIG. 74 is an exploded perspective view of the endoscope of FIG. 69, illustrating the housing assembly having a hollow body and hollow handle extending from the body forming a pistol-grip type handle;

FIG. 75 is a top perspective view of a first side of another exemplary embodiment of an endoscope according to the present disclosure, illustrating a single-use portion that includes a housing assembly with ports extending therefrom, a hub assembly operatively coupled to the housing assembly, a cannula operatively coupled to the hub assembly, an imaging module at a distal part of the cannula, and a cap in a closed position enclosing a wireless reusable portion inserted into an open bottom end of a handle of the housing assembly;

FIG. 76 is a bottom perspective view of the endoscope of FIG. 75, illustrating a single-use portion that includes a housing assembly with a port extending from a top of the housing assembly and a port extending from a bottom of the housing assembly;

FIG. 77 is side elevation view of the first side of the endoscope of FIG. 75, illustrating the ports extending from the top and bottom of the housing assembly, and a sight member extending from a top and bottom of the hub assembly and aligned with a deflection plane of the cannula;

FIG. 78 is a side elevation view of the housing assembly of the endoscope of FIG. 75 with the right housing cover of the housing assembly removed revealing the reusable portion of the endoscope, the deflection control system and the ports extending from the top and bottom of the housing assembly;

FIG. 79 is an exploded perspective view of the endoscope of FIG. 75, illustrating the housing assembly having a hollow body and hollow handle extending from the body forming a pistol-grip type handle;

FIG. 80 is a side view of an endoscope scaled to a selected example, together with a perspective view of a portion thereof and a processing/display unit;

FIGS. 81 and 82 illustrate examples of relative positions of a patient, a camera at the tip of a cannula, a channel in the cannula, and a direction of view of the cannula, and associated displays of such positions;

FIGS. 83a-85b illustrate positions of a deflection place of a cannula and corresponding displays on a screen of a processing/display unit; and

FIGS. 86-89 illustrate images on a display unit for different steps in using an example of an endoscope described in this patent specification to carry out a medical procedure on a patient's kidney.

### SUMMARY OF THE DISCLOSURE

According to some embodiments, an ergonomic ureteroscope with power-assisted, controlled cannula rotation relative to a handle and concurrently operable cannula tip deflection control, comprises: a housing 120, a pistol-grip handle 122 extending down from the housing, a cannula 112 extending distally from the hosing along a cannula axis, a channel 115 through the cannula, and an imaging module 115 at a distal tip of the cannula, wherein the cannula is sufficiently long to reach into an adult patient's kidneys through the patient's urethra and ureter passages; an upper port 194 that is at a proximal upper portion of the housing, in inclined toward the proximal direction, and is configured to communicate with the channel through the cannula, and a lower port 195 that at a lower portion of the housing, is inclined toward the distal direction, and also is configured to communicate with the channel through the cannula; a motorized deflection drive system 130 configured to deflect the distal tip of the cannula relative to the cannula axis in opposed angular directions in a cannula deflection plane, said assembly comprising a motor 152 located in the housing and operatively coupled to the cannula to drive deflection of the cannula and a thumb-operated pivoting lever 240 on each side of the housing for ambidextrous control over the motor to initiate the deflection and to set the deflection angle by pivoting the lever; and a rotation drive system 150 configured to rotate the cannula and thus the cannula deflection plane relative to the housing and comprising a finger operated slider that is at a front side of a proximal portion of housing and is operatively coupled to the cannula to rotate the cannula relative to handle through an angle corresponding to sliding motion of the slider relative to the handle; wherein the deflection drive system and the rotation drive system are configured to enable concurrent operation to rotate the cannula relative to the handle and to deflect the cannula tip relative to the cannula axis.

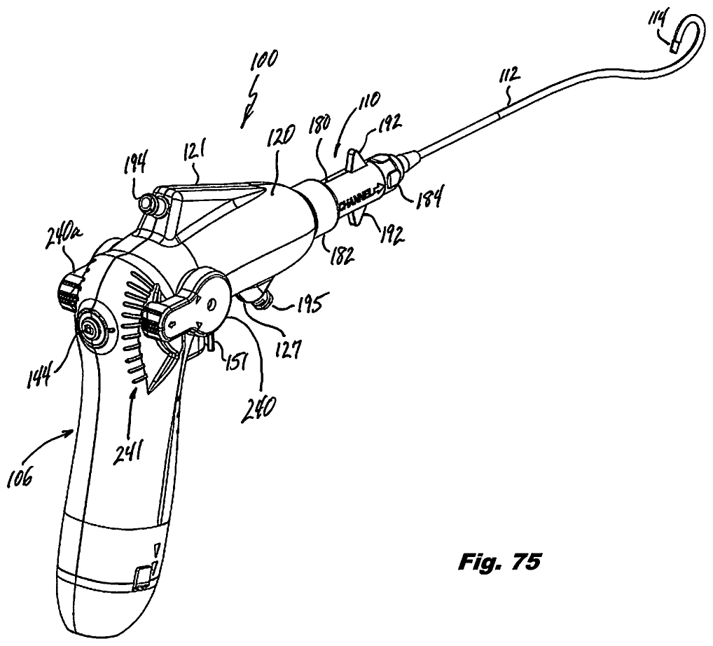

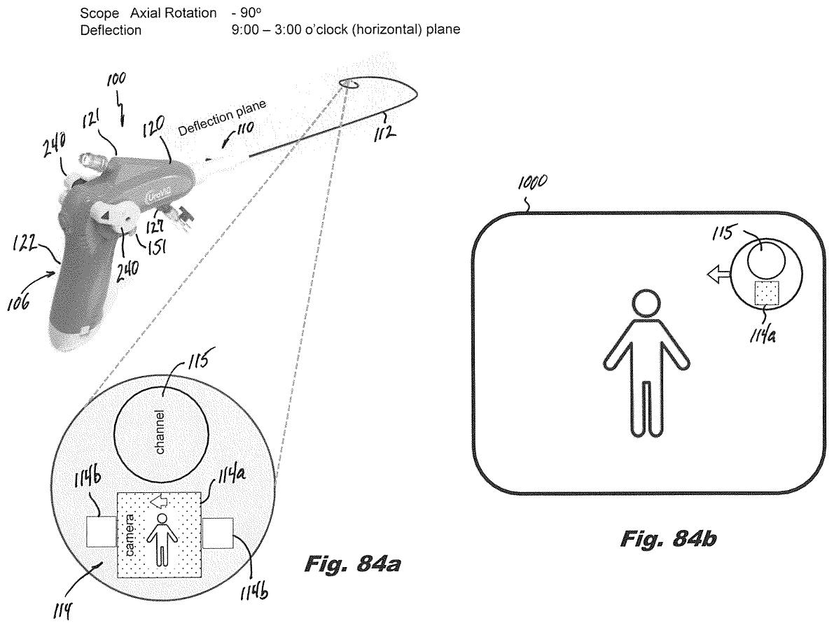

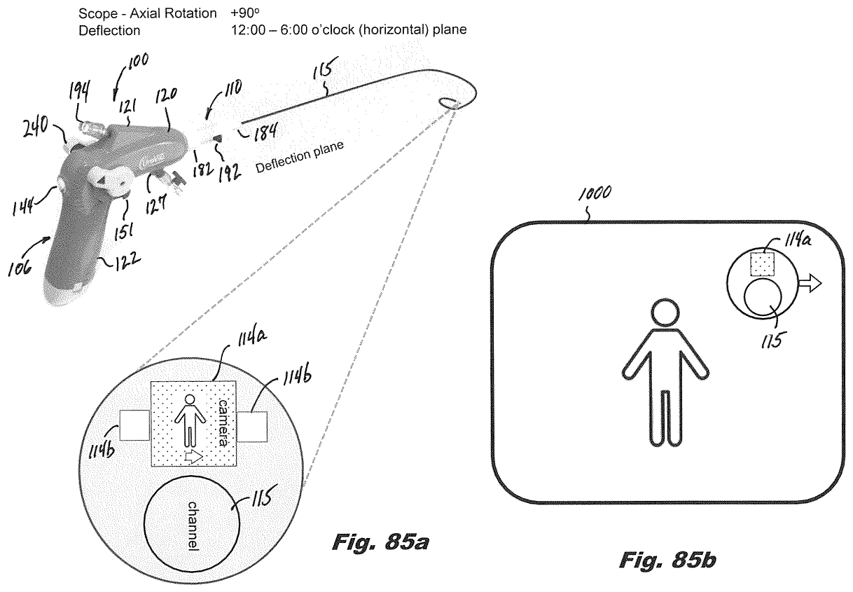

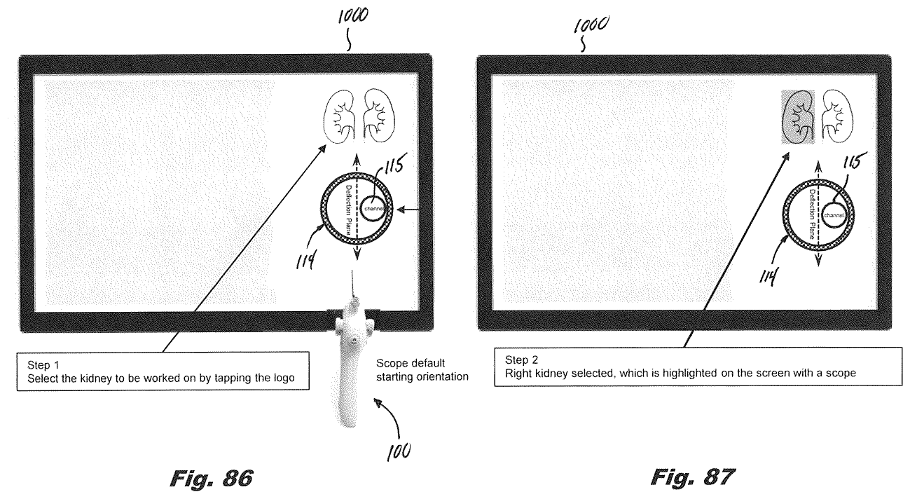

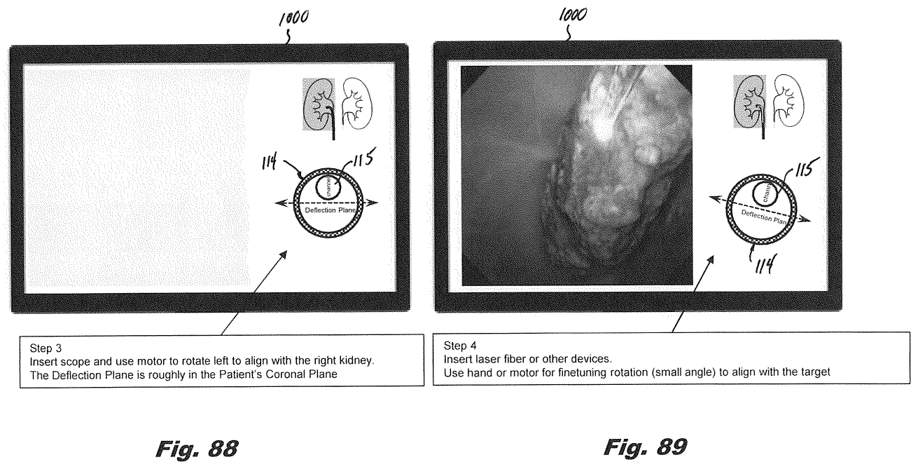

According to some embodiments, the ureteroscope further includes one or more of: (a) an image processor 1000 and display 1002 are operatively coupled with the deflection drive system and with the rotation drive system and are configured to display an image (FIGS. 86-88) that shows a current relative position of the imaging module and the channel and the current direction of view of the imaging module as the cannula rotates relative to the handle and the cannula tip deflects relative to the cannula axis; (b) the image processor and display are further configured to show an icon of a patient (FIGS. 84a-85b) such that the displayed current relative position and direction of view also indicate how the patient's is positioned relative to the imaging module, the channel, and the direction of view; (c) the image processor 1000 and display 1002 are further configured to display icons of left and right kidneys in a first step of a medical procedure and in response to a tap on a selected kidney icon to highlight the kidney icon and to show entry of the cannula tip in the kidney (FIGS. 86-87); (d) the image processor 1000 and display 1002 are further configured to show an image of the kidney interior taken with the imaging module and to respond to rotation of the deflection plane after the cannula tip has entered the kidney to update the displayed current relative position and direction of view; (e) a hub 180 between the housing and the cannula is configured to rotate with the cannula relative to the handle, and a structural indicium (FIG. 75) on a side of the hub is indicative of the relative position of the imaging module and the channel; (f) a sight member 192 extends up from the hub when the hub is at an initial rotation position relative to the handle and rotates with the hub as the cannula rotates relative to the handle; (g) a port extension 121 rising up from the housing 120 and supporting the upper port at a position spaced up from the housing to facilitate insertion of a surgical instrument through the upper port without interfering with the pivoting levers; and (h) structural marks 241 on the housing are adjacent a moving end of the pivoting levers and are configured to facilitate holding the pivoting levers in position and avoid slippage of a finger operating the pivoting levers.

In minimally invasive endoscopic procedures, a clinician typically does not have a direct view of the insertion portion, e.g., the tip and shaft, of the ureteroscope or endoscope that is inserted inside the patient. In addition, clinicians have to control the cannula and instruments that are inserted inside the ureteroscope or endoscope. It is very useful for the operator to be able to track and control the operation of the insertion portion of the ureteroscope or endoscope that is only partially visible. It is also useful to have an ergonomic proximal design of the ureteroscope or endoscope for controlling complex motions of the ureteroscope or endoscope and the surgical instruments. An ureteroscope or endoscope that is able to track and control the operation of the insertion portion of the ureteroscope or endoscope and that includes a single use portion and a reusable portion, significantly reduces the manufacturing cost of such endoscope. Furthermore, an ureteroscope or endoscope with a simple and ergonomic design of the proximal housing assembly and ports, e.g., luer ports, on a hub assembly allows ease of control of the ureteroscope or endoscope and surgical instruments inserted through the ports into a lumen or channel within a cannula of the endoscope. This is particularly important for ureteroscope because the medical procedure is particularly difficult as the distal tip of the ureteroscope needs to navigate through urethra, bladder, ureter and possibly inside the kidney. Ergonomic design of the ureteroscope and ease of use and of control of its functions therefore are especially important for ureteroscopy and pyeloscopy.

The present disclosure provides embodiments of ureteroscope or endoscopes with some or all such features. For example, the present disclosure provides embodiments of single use portions of ureteroscope or endoscopes with a manual deflection control system that deflects the tip of the insertion portion and a motorized or manual rotation drive system that rotates the tip of the insertion portion allowing clinicians to control the rotation and deflection of the tip portion of the insertion portion more efficiently. In some embodiments, the ureteroscope or endoscope includes a manual deflection control system and a motorized rotation drive system to assist the clinician with the particular procedure. In some embodiments, the ureteroscope or endoscope includes a manual deflection control system and a manual rotation drive system to assist the clinician with the particular procedure. And, in some embodiments, the ureteroscope or endoscope includes a manual deflection control system, and the clinician manually rotates the housing assembly during a particular procedure to rotate the tip portion of the ureteroscope or endoscope. For particular ureteroscope or endoscopic procedures, e.g., ureteroscopy, rotation of the ureteroscope or endoscope may be further divided into “Major Rotation” referring to axial rotation of the cannula when the clinician's activates the motorized or manual rotation drive system built into the endoscope or upon the turn of the clinician's hand holding a handle of the housing assembly. “Minor Rotation” refers to subtle rotation of the tip portion of the cannula by the clinician manually rotating a proximal part of the cannula.

In an exemplary embodiment, an endoscope according to the present disclosure is a ureteroscope having a single use portion and a reusable portion that is inserted into the single use portion and encased within the single use portion during a procedure. The single use portion includes a pistol grip housing assembly, a hub assembly, a cannula having one or more channels or lumen extending from its proximal part to its distal part. The cannula ends with a tip portion that includes an imaging module. Within the housing assembly is a motorized rotation drive system and a manual deflection control system. The motorized rotation drive system includes a miniature motor that drives rotation of the cannula, and/or the hub assembly and the cannula. The manual deflection control system includes a lever and a push-pull type mechanism or system that is responsive to the lever. The lever is attached to an exterior of the housing assembly so that the lever is movable relative to the housing assembly. For example, movement, e.g., rotation, of the lever in a first direction pushes the tip portion of the cannula in a first deflection direction, and movement, e.g., rotation, of the lever in a second direction pulls the tip portion of the cannula in a second deflection direction. The second direction may be opposite the first direction. The imaging module may include image optical components such as a camera capable of taking still pictures or generating video images and an illumination source, such as one or more LEDs. The hub assembly may include one or more ports, e.g., two ports, used for the introduction of fluid to the target site within the patient and/or the introduction of surgical instruments to the target site. Generally, the reusable portion is capable of wireless transmission of images from the imaging module to a stand-alone monitor, to a monitor in the procedure room and/or to a processing/display unit. However, the reusable portion may have a wired connection to a stand-alone monitor, to a monitor in the procedure room and/or to a processing/display unit. The reusable portion is located within the housing assembly, e.g., a handle, of the single use portion. The reusable portion may include a rechargeable battery, and one or more printed circuit boards with electronics that facilitate video capture, camera control and wireless transmission functions, such as Wi-Fi transmission.

During a procedure, the ureteroscope or endoscope may interact with an external processing and display unit so that image data and control signals may be exchanged between the ureteroscope or endoscope and the external processing and display unit. Exchange of image data and control signals may be via wireless transmission using a Point-to-Point (PtP) Wi-Fi protocol to provide a secure communication link between connection between the ureteroscope or endoscope and the external processing and display unit. The external processing and display unit may include external connectors, e.g., HDMI connectors, so that image data can be projected to larger displays in the procedure room that are remote from the external processing and display unit.

In some embodiments, plane of deflection of the tip portion of the cannula coincides with the plane formed by the one or more lures ports on the hub assembly. This feature allows the clinician or user to infer the deflection plane of the insertion portion or the distal potion of the cannula that are directly visible inside the patient's body.

In some embodiments, an image flip control switch is provided to allow the displayed image to reflect the orientation of the camera relative to a target within the surgical site. For example, the image flip control switch can be used to allow the displayed image to reflect the orientation of the camera relative to −90° or +90° rotation of the tip portion of the cannula.

In another exemplary embodiment, an endoscope according to the present disclosure is a ureteroscope having a single-use portion, a reusable portion and a cap. The single-use portion includes a housing assembly, a hub assembly, a cannula, an internal channel, a rotation drive system and a deflection control system. The housing assembly has a body and a pistol-grip handle that extends away from the body. The pistol-grip handle has a proximal part that is integral with the housing and an open distal part. The hub assembly extends distally along a cannula axis from the body. The hub assembly includes a body and two proximal ports that extend in different directions transverse to the cannula axis such that at least one of the ports is in a line of sight along the cannula axis from a viewpoint a selected distance proximal from the housing assembly. The cannula extends distally from the hub assembly along the cannula axis and has an imaging module at a distal part. The hub assembly and the cannula are mounted for rotation about the cannula axis relative to the housing assembly body and the cannula is sufficiently long and flexible to reach a kidney of an adult patient when inserted through a urethra, bladder and ureter. The internal channel has a proximal part at the proximal ports and extends therefrom to a distal port at a distal part of the cannula and is configured to provide a path for one or more surgical implements through one of both of said proximal ports to and distally out of the distal part. The rotation drive system is located at the housing assembly and is operatively coupled with the hub assembly to rotate the hub assembly and the cannula about the cannula axis relative to the housing assembly. The rotation drive system includes a manually operated control configured to move relative to the handle to provide a visual and tactile indication of a rotated position of the cannula relative to the handle. The deflection control system is located at the housing assembly and is interactive with the distal part of the cannula, such that when the deflection control system is activated the distal part of the cannula deflects in a positive direction between a first position and a second position along and in a negative direction between the first position and a third position along a deflection plane. The positions of the proximal ports relative to the handle provide an immediate visual and tactile indication of the deflection plane in which the distal part of the cannula is located relative to the orientation of the handle. The reusable portion of the ureteroscope includes a housing and at least one electrical contact. The housing has a proximal part and a distal part and is configured for insertion in the open distal part of the handle. The at least one electrical contact is located at the proximal part of the housing and is accessible from an exterior of the reusable portion housing. The at least one electrical contact is configured to mate with one or more electrical contacts within the handle when the proximal part of the housing is inserted in the handle. The cap of the ureteroscope is configured to releasably mate with the open distal part of the handle so that when the reusable portion is inserted in the open distal part of the handle, the cap covers the open distal part of the handle and encloses the distal part of the reusable portion housing.

In another exemplary embodiment, an endoscope according to the present disclosure is a ureteroscope having a single-use portion, a reusable portion and a cap. The single-use portion includes a housing, a hub assembly, a cannula, a rotation drive system and a deflection control system. The housing assembly has a body and a handle extending away from the housing body. The handle has a proximal part that is integral with the housing and an open distal part. The hub assembly extends distally along a cannula axis from the body. The hub assembly has a body and at least one port extending from the body at a predefined angle relative to the hub assembly body. The cannula extends distally from the hub assembly along the cannula axis and has an imaging module at a distal part. The hub assembly and the cannula are mounted for rotation about the cannula axis relative to the housing assembly body. The cannula has at least one channel extending from a proximal part of the cannula to the distal part of the cannula. The rotation drive system is located within the housing assembly and is operatively coupled with the hub assembly to rotate the hub assembly and the cannula about the cannula axis. The deflection control system is located within the housing assembly and is interactive with the distal part of the cannula, such that when the deflection control system is activated the distal part of the cannula can deflect in a positive direction between a first position and a second position along a deflection plane, or the distal part of the cannula can deflect in a negative direction between the first position and a third position along the deflection plane. The reusable portion of the ureteroscope includes a housing and at least one electrical contact. The housing has a proximal part and a distal part and is configured for insertion in the open distal part of the handle. The at least one electrical contact is located at the proximal part of the housing and is accessible from an exterior of the reusable portion housing. The at least one electrical contact is configured to mate with one or more electrical contacts within the handle when the proximal part of the housing is inserted in the handle. The cap is configured to releasably mate with the open distal part of the handle so that when the reusable portion is inserted in the open distal part of the handle the cap covers the open distal part of the handle and encloses the distal part of the reusable portion housing.

In some embodiments, the rotation drive system includes a powered rotation drive system to rotate the hub assembly and the cannula about the cannula axis. In some embodiments, the powered rotation drive system includes at least one selectively activated motor to rotate the hub assembly and the cannula about the cannula axis.

In some embodiments, the rotation drive system includes a manual rotation drive system to rotate the hub assembly and the cannula about the cannula axis. The manual rotation drive system includes at least one thumbwheel to rotate the hub assembly and the cannula about the cannula axis.

In some embodiments, the deflection control system includes a manually operated deflection control system interactive with the distal part of the cannula. In some embodiments, the manually operated deflection control system includes a lever positioned on an exterior of the housing assembly and at least one push-pull cable operatively connected to the lever and the cannula, such that motion of the lever in a first direction causes the distal part of the cannula to deflect in a positive direction between the first position and the second position, and motion of the lever in a second direction causes the distal part of the cannula to deflect in a negative direction between the first position and the third position. In some embodiments, the manually operated deflection control system includes at least one deflection wheel lever positioned on an exterior of the housing assembly and at least one push-pull cable operatively connected to the deflection wheel lever and the cannula, such that motion of the deflection wheel lever in a first direction causes the distal part of the cannula to deflect in a positive direction between the first position and the second position, and motion of the deflection wheel lever in a second direction causes the distal part of the cannula to deflect in a negative direction between the first position and the third position.

In some embodiments, the reusable portion includes a battery and control and processing electronics configured to control the imaging module to take images in a field of view and to receive image data from the imaging module. In some embodiments, the reusable portion includes a facility to convey image data from the endoscope to an external processing/display unit. In some embodiments, the facility is configured to convey image data by wireless transmission using a Point-to-Point Wi-Fi protocol. In some embodiments, the facility in the reusable portion is configured to convert received image data into display images and convey display images to an external unit for display.

In some embodiments, the ureteroscope according to the present disclosure may include a manual switch at the distal part of the reusable portion, and at least the portion of the cap that is over the switch is sufficiently flexible for manual operation of the switch through the cap.

### DETAILED DESCRIPTION

A detailed description of examples of preferred embodiments is provided below. While several embodiments are described, the new subject matter described in this patent specification is not limited to any one embodiment or combination of embodiments described herein, but instead encompasses numerous alternatives, modifications, and equivalents. In addition, while numerous specific details are set forth in the following description to provide a thorough understanding, some embodiments can be practiced without some or all such details. Moreover, for the purpose of clarity, certain technical material that is known in the related art has not been described in detail in order to avoid unnecessarily obscuring the new subject matter described herein. It should be clear that individual features of one or several of the specific embodiments described herein can be used in combination with features of other described embodiments or with other features. Further, like reference numbers and designations in the various drawings indicate like elements.

As described in detail below, a ureteroscope or endoscope according to a preferred embodiment is essentially self-contained, communicating with a processing/display unit wirelessly, for example via Wi-Fi or a near field link, but can have a port for a cable connection to a processing/display unit in case a wireless connection is not available or desirable at a medical site. The ureteroscope or endoscope may include a power source, e.g., a battery, as well as sufficient electronics to control an imaging module at the distal part of a cannula and for processing image data from the imaging module into images for display so that only minimal control and/or processing is required at an external display to show the images. In other preferred examples, the processing/display unit can contain facilities to control some or all the functions of the imaging module and to do some or all the processing of image data from the imaging module for display.

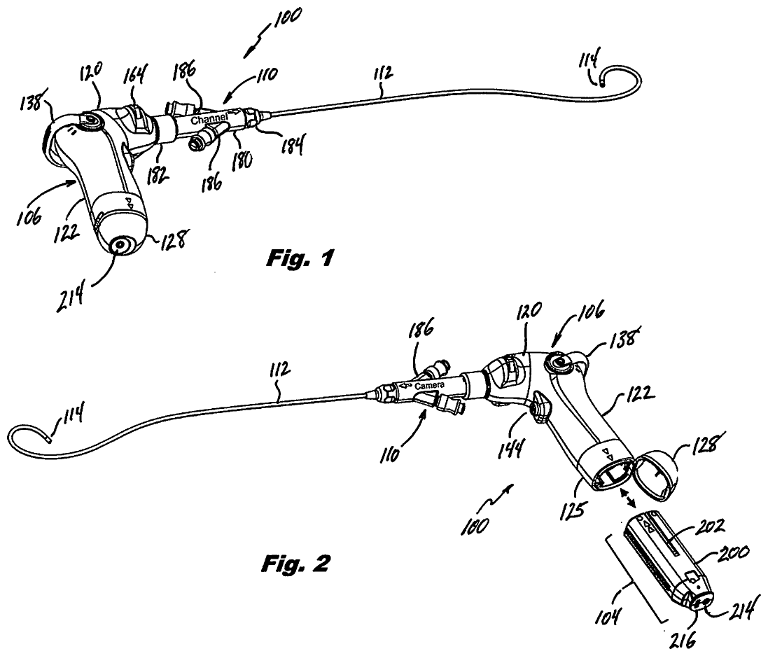

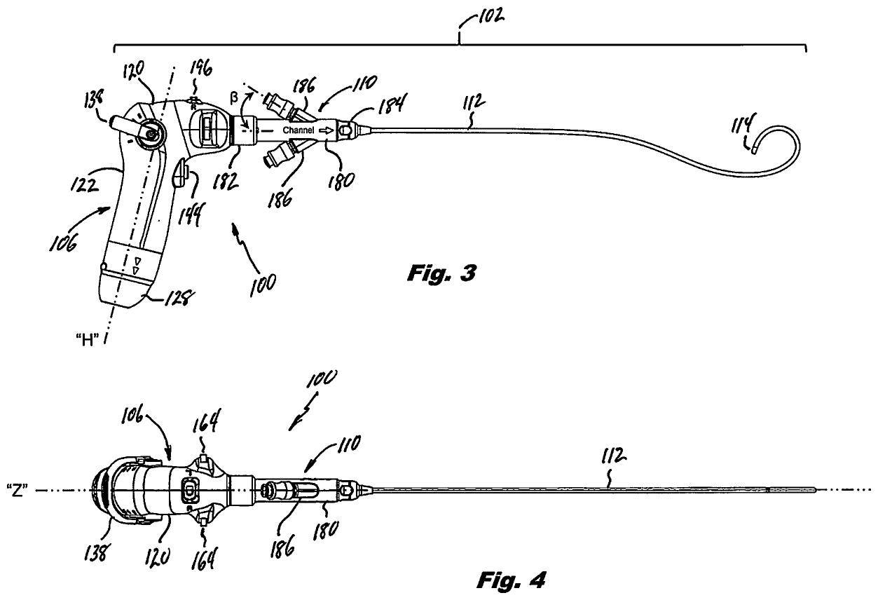

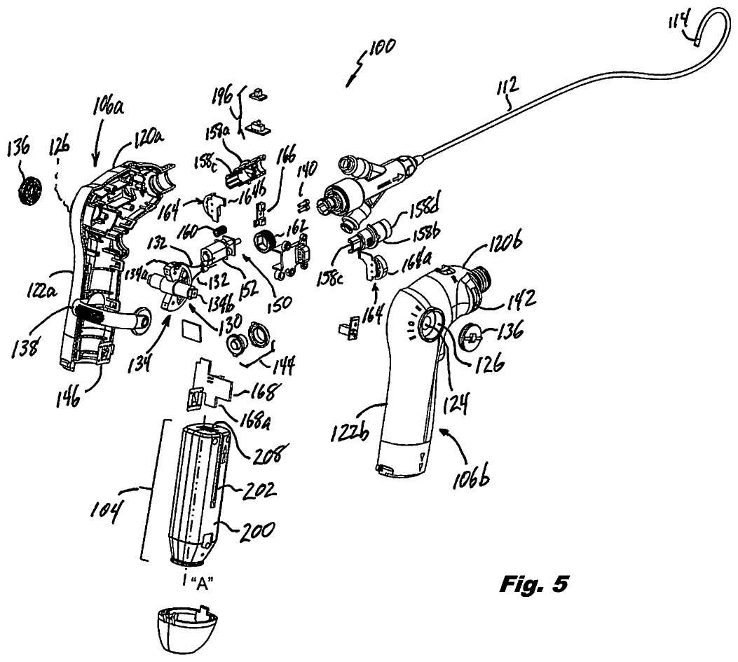

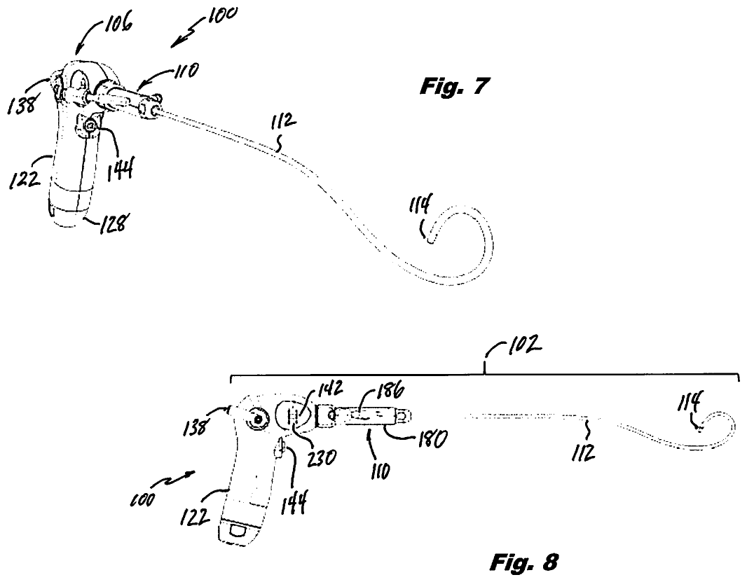

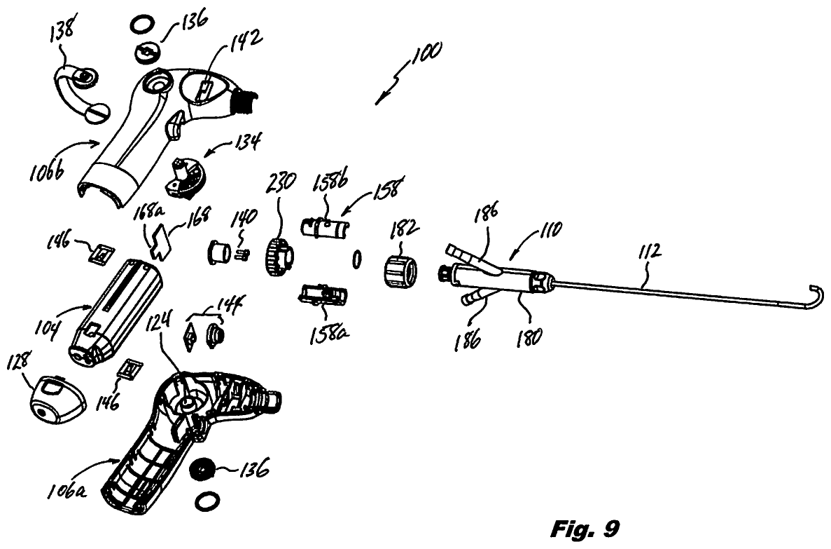

FIGS. 1-4 are various views of an exemplary embodiment of an ureteroscope or endoscope 100 according to the present disclosure, and FIG. 5 is an exploded view of the ureteroscope or endoscope 100. Ureteroscope 100 includes a single-use portion 102 and a reusable portion 104 that when assembled form the ureteroscope 100. The single-use portion 102 includes a housing assembly 106, a hub assembly 110, a cannula 112 and an imaging module 114. In the exemplary embodiment shown, housing assembly 106 includes a body 120, a handle 122 and a cap 128. The body 120, handle 122 and cap 128 are preferably made of a plastic material, such as a thermoplastic material. Non-limiting examples of such thermoplastic materials include polycarbonate (PC), and a combination of polycarbonate (PC) and acrylonitrile-butadiene-styrene terpolymer (ABS). The body 120 is a hollow member that extends along a cannula axis “Z”, seen in FIG. 4. The handle 122 is a hollow, pistol-grip type handle that extends from the body 120 preferably along a handle axis “H” (FIG. 3) that is at an angle relative to a longitudinal axis of the body 120. The handle 122 has a proximal part that is integral with or monolithically formed into the body 120 and has an open distal part 125, seen in FIG. 2. The hollow portion of the handle 122 is configured and dimensioned to receive the reusable portion 104 as described in more detail below. The cap 128 may be removably secured to the distal part 125 of the handle 122 to close the open distal part 125 of the handle 122. In the exemplary embodiment shown in FIGS. 1-5, the cap 128 is secured to the distal part 125 of the handle 122 by a hinge, e.g., a living hinge or a mechanical hinge, such that the cap 128 is pivotable between an open position permitting access to the open distal part 125 of the handle 122 and a closed position preventing access to the open distal part 125 of the handle 122. In the exemplary embodiment shown in FIGS. 7-10, cap 128 is removably secured to the distal part 125 of the handle 122 by a snap-fit connection. It is noted that when the reusable portion 104 is properly inserted into the ureteroscope 100, a part of the reusable portion 104 may protrude distally from handle 122. When the cap 128 is in the closed position, the cap 128 covers not only the part of reusable portion 104 that protrudes distally from the handle 122, but also an adjacent part of the distal part 125 of the handle 122 to seal the interface between the open end of handle 122 and reusable portion 104. It is also noted that for ease of manufacture, the body 120 and handle 122 of the housing assembly 106 may be a two-piece structure having a left housing cover 106a and a right housing cover 106b, seen in FIG. 5, that are joined together by for example, ultrasonic welds, adhesives or mechanical connections, such as snap-fit connections. In such a configuration, the left housing cover 106a and the right housing cover 106b (FIG. 5) are substantially the same except for orientation so that the left housing cover 106a can be attached to the right housing cover 106b top form the housing assembly 106.

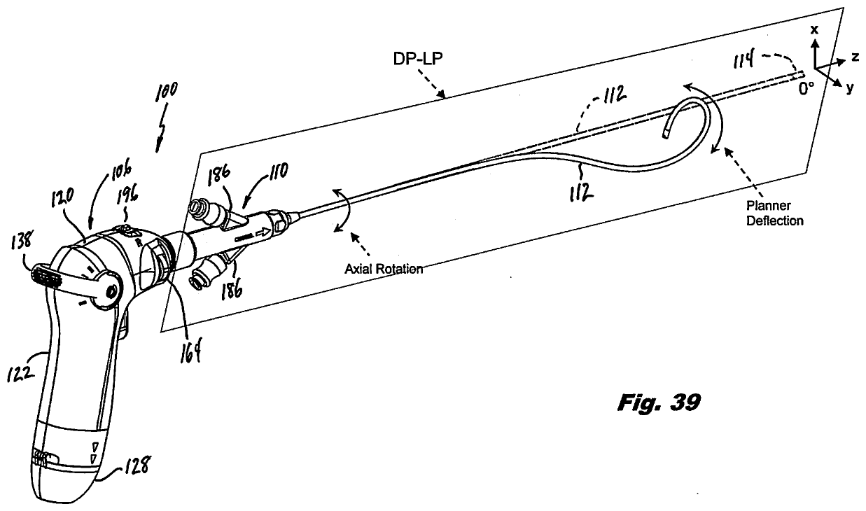

Within housing assembly 106 is a deflection control system 130 and a rotation drive system 150. The deflection control system 130 is provided to deflect or bend the distal part of the cannula 112 within a deflection plane, seen in FIGS. 39-41, between a first full deflection position and a second full deflection position. A non-limiting example of a range of deflection for the distal part of the cannula −270 degrees and +270 degrees. In the exemplary embodiment shown, the deflection control system 130 includes one or more cables or wires 132, a cable wheel 134, one or more lever wheels 136 and a level 138, seen in FIG. 5. The one or more cables or wires 132 pass through a cable housing 140 and through the hub assembly 110 to the distal part of the cannula 112 as is known. The free ends of the one or more cables 132 are attached to the cable wheel 134. The cable wheel 134 is an arcuate shaped member 134a having an arm 134b that is transverse to the arcuate shaped member. An end of the arm 134b associated with the left housing cover 106a (the “left arm end”) passes through an aperture 124 in the left housing cover 106a, and the end of the arm 134b associated with the right housing cover 106b (the “right arm end”) passes through an aperture 124 in the right housing cover 106b. A left lever wheel 136 is attached to the left arm end and rests within a recess 126 in an exterior of the left housing cover 106a. A right lever wheel 136 is attached to the right arm end and rests within a recess 126 in an exterior of the right housing cover 106b. A lever 138 is then attached to the left and right lever wheels 136 as shown in FIGS. 1, 2 and 4 so that the lever 138 is rotatable relative to the housing assembly 106.

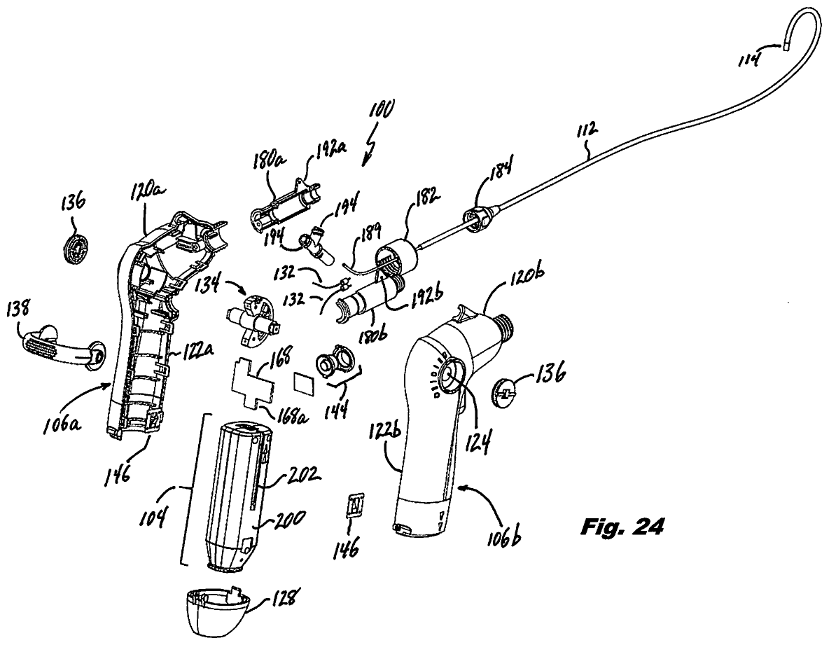

Continuing to refer to FIG. 5, the rotation drive system 150 includes a motor 152 mounted to a motor base board 164, a gear assembly attached to the drive shaft of the motor and to a proximal part of a motor adapter 158 formed of portions 158a-d. The gear assembly reduces the rate of rotation of the motor drive shaft. As an example, motor 152 may be configured to rotate the motor drive shaft at a rate in the range of about 17 rpm. In this configuration, the battery voltage may be in the range of about 3.7 VDC. The gear assembly may reduce the rate of rotation of the motor drive shaft to a rate below 17 rpm as desired. In the exemplary embodiment shown, the gear assembly includes a first gear 160 attached to the motor drive shaft that meshes with a second gear 162 such that rotation of the first gear 160 causes rotation of the second gear 162 at a slower rate. The motor adapter may be a separate component or the motor adapter may be part of the hub assembly 110. In the exemplary embodiment shown in FIG. 5, the motor adapter is a separate component. For ease of manufacture, the motor adapter 158 may be a two-piece structure having a left adapter cover 158a and a right adapter cover 158b that are joined together by for example, ultrasonic welds, adhesives or mechanical connections, such as snap-fit connections. The motor adapter has a gear shaft 158c at a proximal part configured and dimensioned to receive the second gear 160 such that rotation of the second gear is transferred to rotation of the motor adapter. A hub interface 158d of the motor adapter is operatively connected to the hub assembly 110 such that rotation of the motor adapter is translated to rotational movement of the hub assembly 110. To activate the motor 152 and limit the rotation of the motor adapter, the rotation drive system 150 also includes a pair of motor switches 164 and a limit switch 166. One of the pair of motor switches 164 is secured within the left body 120a of the left housing cover 106a so that an actuating arm 164a of the motor switch 164 extends through an opening 142 in the left body 120a. Similarly, the other motor switch 164 is secured within the right body 120b of the right housing cover 106b so that an actuating arm 164a of the motor switch 164 extends through an opening 142 in the right body 120b, as shown in FIGS. 1 and 2, enabling ambidextrous operation. The pair of motor switches 164 are electrically connected to the motor base board 154 via one or more electrical contacts 164b, and the motor base board 154 is electrically connected to printed circuit board (PCB) 168 within the handle 122 via internal wires (not shown). Electrical contacts 168a of the printed PCB 168 are electrically connected to electrical contacts 208 within reusable portion 104. Control and processing electronics 210 within the reusable portion 104 may control the operation of the motor 152 in response to clinician actuation of the left or right motor switches 164. The limit switch 166 shuts the motor 150 off in the event the axial rotation of the motor adapter 158 or hub assembly 110 reaches a predefined range of axial rotation. As a non-limiting example, the predefined range of axial rotation of the motor adapter 158 or hub assembly 110 may be between about −150 degrees and about +150 degrees, such that if the motor adapter 158 or hub assembly 110 rotates to about −150 degrees the limit switch 166 shuts off the motor 150, and if the motor adapter 158 or hub assembly 110 rotates to about +150 degrees the limit switch 166 shuts off the motor 150. However, other ranges are contemplated by the present disclosure. For example, using particular types of materials for the cannulas 112 and/or semi-rigid tube(s) 189 (FIG. 24), it may be important that the integrity of the semi-rigid tube(s) 189 and/or the channel 115 (FIG. 48) of the cannula 112 is maintained so that surgical instruments and fluid can pass readily without affecting the handling and/or operations of the ureteroscope 100. In such instances, it may be important to limit the range of axial rotation of the motor adapter 158 or hub assembly 110 to between about −135 degrees and about +135 degrees so that the semi-rigid tube(s) 189 and/or the channel in the cannula 112 are not overly twisted when the motor adapter 158 or hub assembly 110 is rotated. It is noted that the semi-rigid tubes 189 may be, for example, Teflon tubes with an inside diameter of about 1.2 mm and 0.1 mm wall thickness.

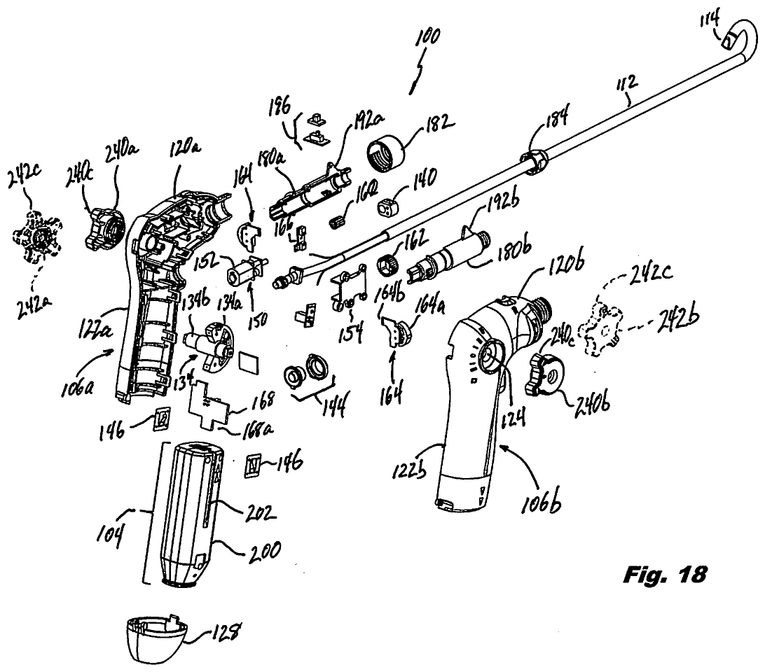

Continuing to refer to FIGS. 1-5, the hub assembly 110 extends distally from the body 120 of the housing assembly 110 along the cannula axis “Z”. The hub assembly 110 includes a hollow body 180 having a housing adapter 182 at a proximal part and a cannula adapter 184 at a distal part. For ease of manufacture, the body 180 may be a two-piece structure having a left body cover 180a and a right body cover 180b, seen in FIG. 18, that are joined together by, for example, ultrasonic welds, adhesives or mechanical connections, such as snap-fit connections. The housing adapter 182 is secured to the body 120 of the housing assembly 106 via a threaded nut rotatably secured to a proximal part of the body 180. The cannula adapter 184 is configured to secure the cannula 112 to the hub assembly 110. The hub assembly 110 may also include one or more ports or connectors 186. The one or more ports or connectors 186 may be luer ports or connectors and for ease of description, the one or more ports or connectors 186 may also be referred to herein as the luer ports. In the exemplary embodiment shown in FIGS. 1-5, there are two luer ports 186 extending from the body 180 preferably at an angle “β” relative to the longitudinal axis of the body 180. The angle may be in the range of about 30 degrees. The one or more luer ports 186 are in communication with the channel 115 in the cannula 112, seen in FIG. 48, so that a surgical instrument or fluid introduced into the luer port 186 can pass into the channel 115 toward the distal part of the cannula 112.

The cannula 112 is a flexible member that is removably coupled to the hub assembly 110 via the cannula adapter 184 and extends distally from the hub assembly 110 along the cannula axis “Z”. It may have sufficient length to reach the kidney of an adult by passing though the urethra and ureter. The imaging module 114 is attached to, formed into or integral with the distal part of the cannula 112. The imaging module 114 may have a housing that includes, as seen in FIG. 48, a camera 114a and one or more illumination sources 114b, e.g., LEDs, that fit in respective openings in the housing formed into or secured at the distal part of cannula 112 so that the camera 114a and LEDs 114b face in the distal direction. The camera 114a and LEDs 114b can be configured to have a field of view and a direction of illumination having a central axis that is at an angle relative to cannula axis “Z”, for example a 30-degree angle. A more detailed description of the imaging module is described in commonly owned U.S. Pat. No. 11,771,304, which is incorporated herein in its entirety.

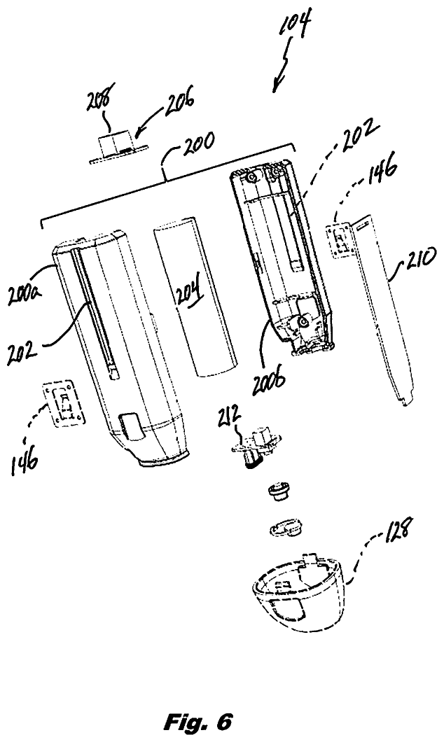

Referring to FIGS. 2, 5 and 6, the reusable portion 104 is elongated along a longitudinal axis “Z,” which also is called cannula axis or long axis, and is configured for insertion into the open distal part 125 of the handle 122. At a proximal part, the reusable portion 104 has one or more electrical contacts 208, seen in FIG. 6, that mate with one or more electrical contacts 168a, seen in FIG. 5, of the PCB 168 within the handle 122 when the reusable portion 104 is inserted all the way into handle 122. Internal wires or cables (not shown) connect the camera 114a and LED's 114b of the imaging module 114 to the PCB 168. For example, the internal wires can provide power to the imaging module 114 and to transfer image data from the imaging module 114 to the PCB 168.

Notably, to ensure ease of assembling the ureteroscope 100 and correctly fitting reusable portion 104 into handle 122, the handle and the reusable portion are configured or shaped such that the reusable portion 104 can be inserted into the handle 122 in only one orientation. For example, as seen in FIGS. 2, 5 and 6, the handle 122 may include one or more alignment clips 146 and a housing 200 of the reusable portion 104 may include one or more grooves or channels 202 such that when the one or more grooves or channels 202 are aligned with the one or more alignment clips 146, the insertion orientation of the reusable portion 104 is correct. With the correct insertion orientation, the reusable portion 104 can be inserted all the way into handle 122 so that the one or more electrical contacts 168a of the PCB 168 plug into the one or more electrical contacts 208 of the PCB 206. If the one or more alignment clips 146 are not properly aligned with the one or more grooves or channels 202, the reusable portion 104 is prevented from being inserted all the way into the handle 122.

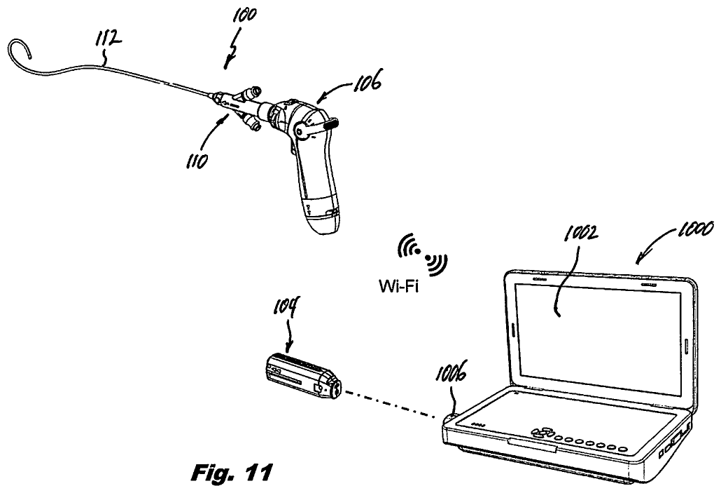

Referring to FIGS. 6 and 11, the reusable portion 104 includes the housing 200 defined by a first housing portion 200a and a second housing portion 200b that when joined form the housing 200. The first housing portion 200a and a second housing portion 200b can be joined by, for example, ultrasonic welds, adhesives or mechanical fasteners. A non-limiting example of mechanical fasteners include snap-fit connections. Exterior surfaces of the first housing portion 200a and the second housing portion 200b may include the grooves or channels 202 described above. The reusable portion 104 may include a rechargeable battery 204 within the housing 200 that is in electrical communication with a printed circuit board (“PCB”) 206 within the housing 200 via internal wires (not shown). The PCB 206 includes the one or more electrical contacts 208 that mate with the one or more electrical contacts 168a of the printed PCB 168 within handle 122 of the housing assembly 106. Preferably, electrical contacts 208 include power contacts and data contacts to provide power to the PCB 168 and data communication lines to the PCB 168. Electrical power at the PCB 168 can then be provided to the power imaging module 114. In instances where a photograph or a video clip of the target area within a patient is desired, a switch 144 within the housing assembly 106 of the single-use portion 102 and accessible from an exterior of the housing assembly 106 may be used. More specifically, the photo switch 144 may be electrically connected to the imaging module 114 via internal wires (not shown) in the single-use portion 102, so that when the clinician activates the photo switch 144, the camera 114a of the imaging module 114 snaps a photo or video of the target area. Electrical power at the PCB 168 can also be provided to the motor 152 through motor switches 164 within the housing assembly 106 of the single-use portion 102. Image data, e.g., video and photo data, from the imaging module 114 can be sent to the PCB 168 via internal wires (not shown) and to the PCB 206 via the data communication lines referenced above. The reusable portion 104 may also include a printed circuit board with the control and processing electronics 210 configured to communicate with the imaging module 114 and the motor 152 of the rotation drive system 150 via the electrical contacts 208. The control and processing electronics 210 may include a processor and related circuitry to control the operation of the imaging module 114, to process image data received from the imaging module 114 and to control the operation of the motor 152 in response to actuation of the motor switches 164. According to some embodiments, the control and processing electronics 210 can include Wi-Fi, near-filed or other wireless facility to communicate with an external processing/display unit 1000, seen in FIG. 11, and facilities to fully or nearly fully control the operation of imaging module 114 through suitable switches, and to fully or nearly fully process the image data received from the imaging module 114 into images for display so that the external processing/display unit 1000 needs to do only minimal processing of image data or of display images. The Wi-Fi, near-field or other wireless facility can also communicate with the external processing/display unit 1000 to facilitate full or nearly full control the operation of the motor 152 of the of the rotation drive system 150 through suitable switches. According to some embodiments, some or all of the control over the imaging module 114, some or all the processing of image data from the imaging module 114 and/or some or all of the control over the motor 152 can be done by or at the external processing/display unit 1000.

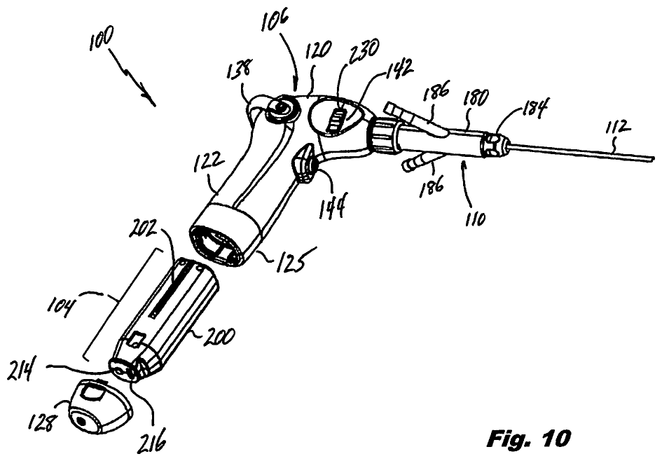

Continuing to refer to FIGS. 6 and 11, the reusable portion 104 may also include a printed circuit board with electronics 212 providing a power on/off switch 214 and a battery charging connector 216 (FIG. 10). The switch 214 turns power to the reusable portion 104 ON or OFF and thus power to the single-use portion 102 ON or OFF. The charging connector 216 is electrically connected to the rechargeable battery 204 so that the battery 204 can be connected to the external processing/display unit 1000 for recharging, as shown in FIG. 11. Non-limiting example of charging connectors 216 include Type C and Thunderbolt connectors.

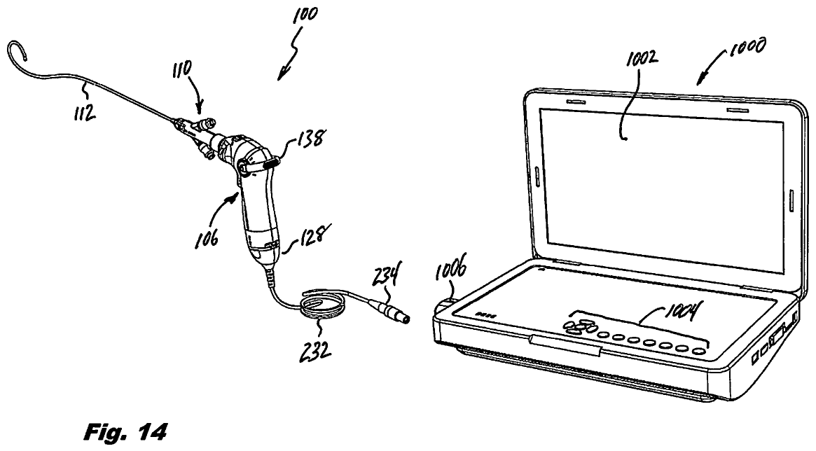

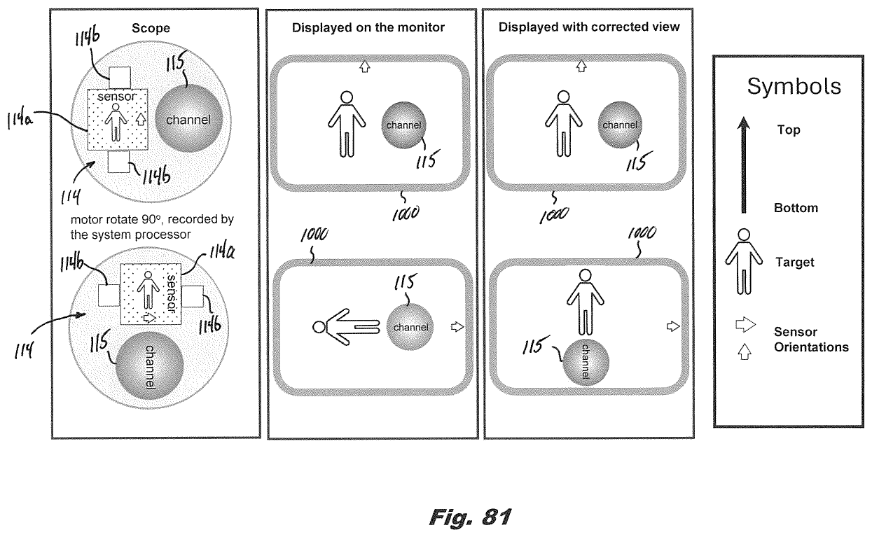

For ease of description, the external processing/display unit 1000 may also be referred to herein as the PD unit 1000 or an image processor. In an exemplary embodiment, the PD unit 1000 includes a display 1002, one or more switches or keys 1004 (FIG. 14) and/or an adapter or connectors 1006. The display 1002 is configured to display image data received from the camera 114a in the imaging module 114 and/or other data and images. For example, while displaying image data received from the camera 114a, the PD unit 1000 can be configured to overlay an image of the tip portion of the cannula 112 showing a real-time view of the orientation of the camera 114a and channel 115 during the procedure, as shown in FIGS. 81-8991. The one or more switches or keys 1004 can be configured to control functions such as operation of the imaging module 114 and the motor 152 of the ureteroscope 100 and the PD unit 1000. The adapter or connectors 1006 can be provided to, for example, connect the reusable portion 104 to the PD unit 1000 for charging the battery 204 in reusable portion 104. The adapter or connectors 1006 of the PD unit 1000 may also include one or more cable connectors, e.g., HDMI connector or other high-speed connectors, for a data link to an external display (not shown) that may be a large and/or high-definition monitor or a workstation. A wireless link also can be used for a connection of the ureteroscope 100 and/or PD unit 1000 with another device, such as a smartphone or a tablet or a workstation. The wireless link can be Wi-Fi link, or a Point-to-Point (PIP) Wi-Fi link, or can use a near filed communication protocol (NFC) or another protocol. According to some embodiments, the wireless link can be configured such that transmission between the ureteroscope 100 and PD unit 1000 is automatically established. The PD unit 1000 may be configured such that after being turned ON, the PD unit searches for wireless endoscopes 100 that are within range of the PD unit 1000. Once the PD unit 1000 finds endoscopes 100 that are within range, the PD unit 1000 can be configured to automatically connect to one or more endoscopes 100 within range for receiving and transmitting wireless data. This transmission can include image and/or other data from the one or more endoscopes 100 within range of the PD unit and/or commands and/or other information from PD unit 1000.

Referring now to FIGS. 7-10, another exemplary embodiment of an ureteroscope 100 according to the present disclosure is shown. In this exemplary embodiment, many of the components are designated by reference numerals discussed above and are the same as or similar to such like components and serve the same or similar functions as such like components. However, in this exemplary embodiment, the rotation drive system 150 differs. More specifically, the rotation drive system 150 is not motorized. Instead, the rotation drive system 150 is a manual system that includes a thumbwheel 230 that is secured to the proximal part of the body 180 of the hub assembly 110 such that rotation of the thumbwheel 230 causes the hub assembly 110 and thus the cannula 112 to rotate relative to handle 122. In order to rotate the thumbwheel 230, a portion of the thumbwheel 230 passes through the openings 142 in the left body 120a and the right body 120b of the housing assembly 106. To rotate the cannula 112 the clinician rotates the thumbwheel 230 either clockwise or counter-clockwise. It is noted that in this exemplary embodiment, the luer plane (LP), which is described below, is orthogonal to the deflection plane (DP), which is described below.

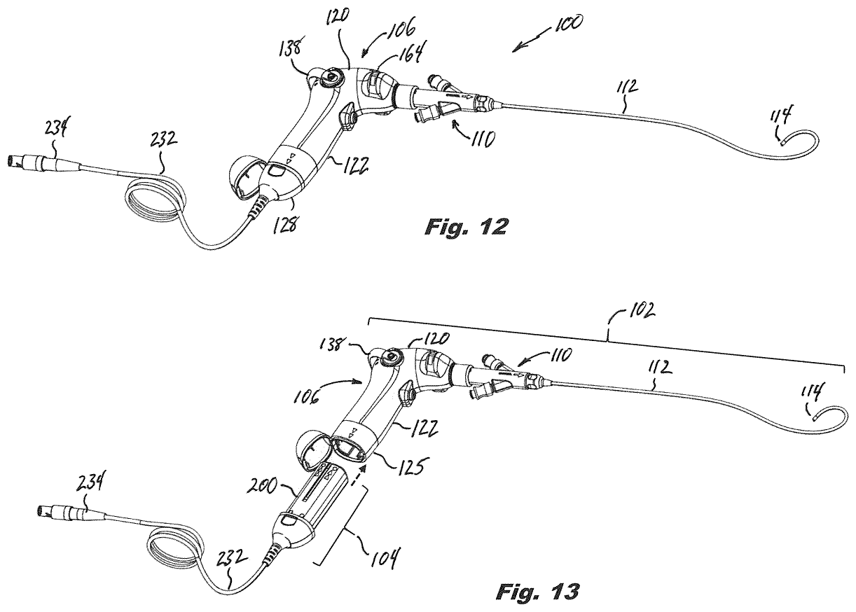

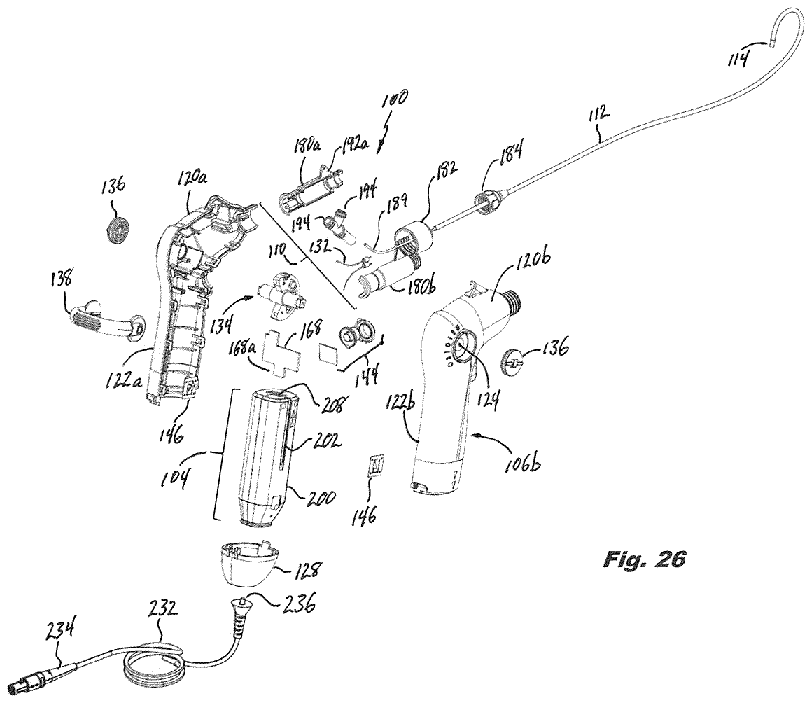

Referring to FIGS. 12-14, another exemplary embodiment of a ureteroscope 100 according to the present disclosure is shown. In this exemplary embodiment, many of the components are designated by reference numerals discussed above and are the same as or similar to such like components and serve the same or similar functions as such like components. However, in this exemplary embodiment, the reusable portion 104 differs. More specifically, the reusable portion 104 communicates with the external processing/display unit 1000, seen in FIG. 14, via a cable connection. In this exemplary embodiment, cable 232 has a proximal part that includes one or more electrical contacts 236 (FIG. 26) passed through an opening in the cap 128 and plugged into the charging connector 216 at the bottom end of the reusable portion 104. The distal part of the cable 232 includes a connector 234 that can be plugged into a suitable connector 1006 on the PD unit 1000. Operation of the ureteroscope 100 of FIGS. 12-14 is the substantially the same as the embodiments of the endoscope described herein, except that data is exchanged with the PD unit 1000 via cable 232. The battery 204 can also be recharged via cable 232.

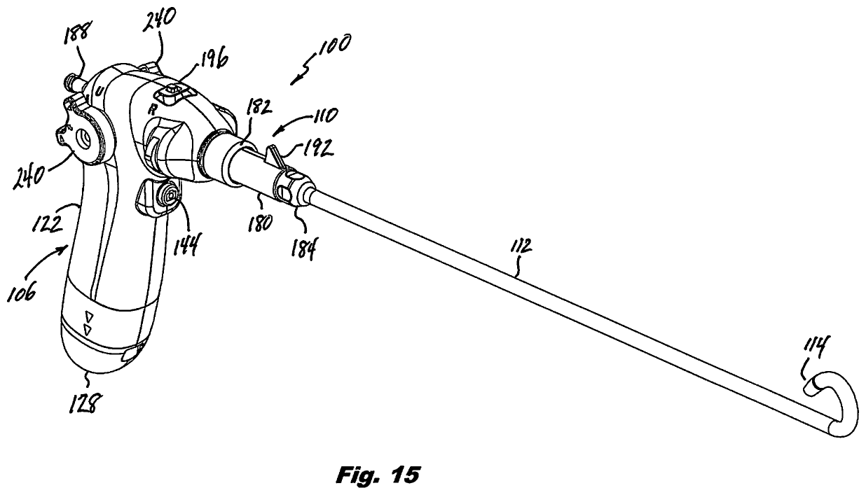

Referring now to FIGS. 15-18, another exemplary embodiment of a ureteroscope 100 according to the present disclosure is shown. In this exemplary embodiment, many of the components are designated by reference numerals discussed above and are the same as or similar to such like components and serve the same or similar functions as such like components. However, in this exemplary embodiment, the lever of the deflection control system 130 is different, a distal luer port 188 is included at a proximal part of the body 120 of the housing assembly 106 and a sight member 192 is position on the body 180 of the hub assembly 110. In this exemplary embodiment, the deflection control system 130 includes a pair of deflection wheel levers 240 or 242 (shown in phantom) attached to arm 134b of the cable wheel 134. More specifically, a left deflection wheel lever 240a or 242a (shown in phantom) is attached to the end of the arm 134b passing through the aperture 124 in the left housing cover 106a, and a right deflection wheel lever 240b or 242b (shown in phantom) is attached to the other end of the arm 134b passing through the aperture 124 in the right housing cover 106b. The left and right deflection wheel levers 240 or 242 are rotatable relative to the housing assembly 106 such that clockwise and counter-clockwise rotation of the left and/or right deflection wheel levers 240 or 242 causes the hub assembly 110 and cannula 112, and thus the imaging module 114, to deflect in the deflection plane (DP), seen in FIGS. 39-41, between the first full deflection position and the second full deflection position described above. It is noted that the deflection wheel levers 240 have two ribs 240c extending therefrom, seen in FIG. 18, and the deflection wheel levers 242 (shown in phantom) have five ribs 242c extending therefrom, seen in FIG. 18. However, the deflection wheel levers may have a single rib or any other number of ribs. The ribs facilitate easy rotation of the deflection wheel levers 240 or 242 by a clinician.

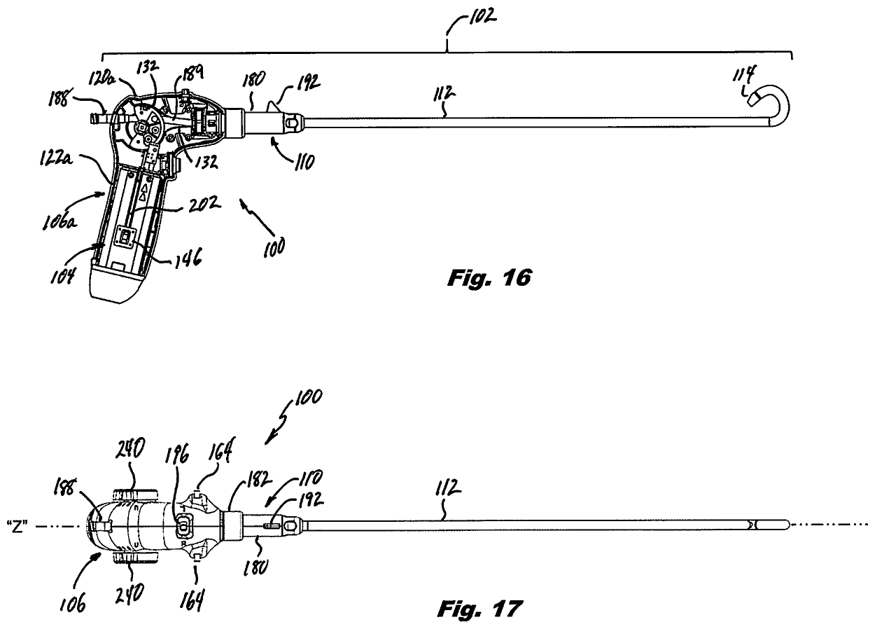

Continuing to refer to FIGS. 15-18, the proximal luer port 188 is secured to the proximal part of the body 120 of the housing assembly 106 so that part of the luer port 188 extends away from the body 120 substantially along the cannula axis “Z”, seen in FIG. 16. The luer port 188 is in communication with the channel 115 in the cannula 112 so that surgical instruments or fluid can be passed from the luer port 188, through the channel 115 and exit the distal part of the cannula 112. As a non-limiting example, the luer port 188 can be connected to the cannula channel 115 via a semi-rigid tube 189, seen in FIG. 16. In this exemplary embodiment, the luer port 188 remains fixed relative to the body 120 of the housing assembly 106.

Continuing to refer to FIGS. 15-18, to provide the clinician with the location of the deflection plane (DP) of the cannula 112, the hub assembly 110 includes the sight member 192 positioned on the body 180 of the hub assembly 110 so that the sight member 192 is aligned with the deflection plane (DP) of the cannula 112. As the hub assembly 110 is rotated by, for example, the rotation drive system 150, the sight member 192 rotates providing the clinician a visual indication of the deflection plane (DP) of the cannula 112. Sight member 192 can be colored in a readily visible color that contrasts with other colors typically found in medical procedure locations, such as black.

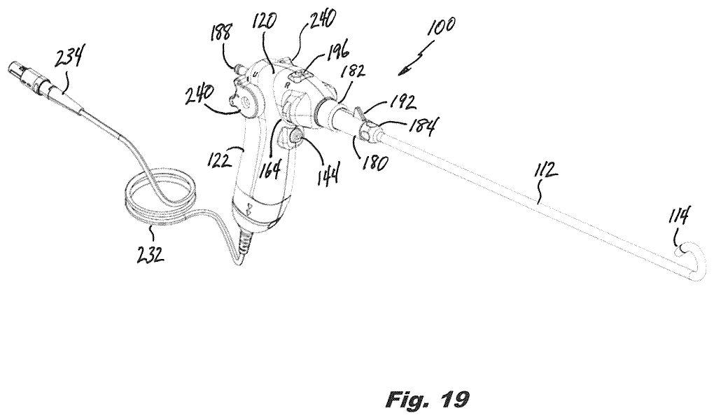

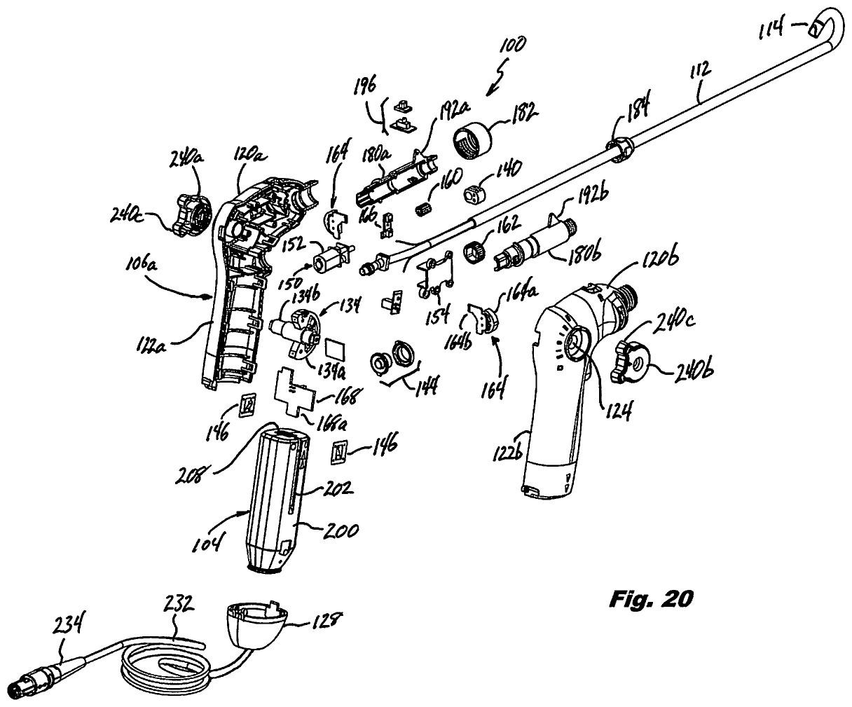

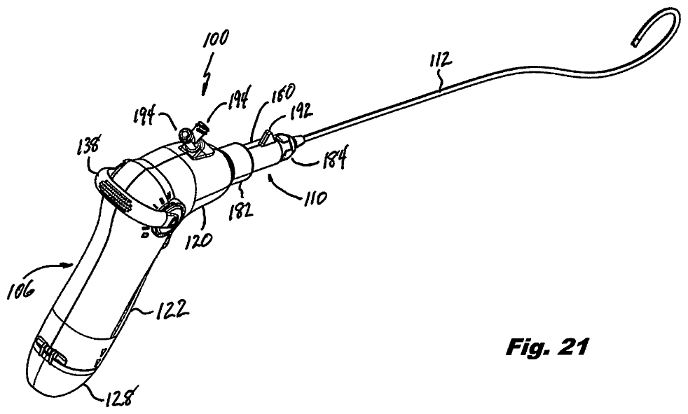

Referring now to FIGS. 19 and 20, another exemplary embodiment of a ureteroscope 100 according to the present disclosure is shown. In this exemplary embodiment, many of the components are designated by reference numerals discussed above and are the same as or similar to such like components and serve the same or similar functions as such like components. However, in this exemplary embodiment, the reusable portion 104 differs. More specifically, the reusable portion 104 communicates with the external processing/display unit 1000, seen in FIG. 11, via a cable connection. In this exemplary embodiment, cable 232 has a proximal part that includes one or more electrical contacts 236 passed through an opening in the cap 128 and are plugged into the charging connector 216 (FIG. 10) at the bottom end of the reusable portion 104. The distal part of the cable 232 includes a connector 234 that can be plugged into a suitable connector 1006 on the PD unit 1000. Operation of the ureteroscope 100 of FIGS. 19-22 is substantially the same as or similar to the embodiments of the endoscope described herein, except that data is exchanged with the PD unit 1000 via cable 232. The battery 204 can also be recharged via cable 232.

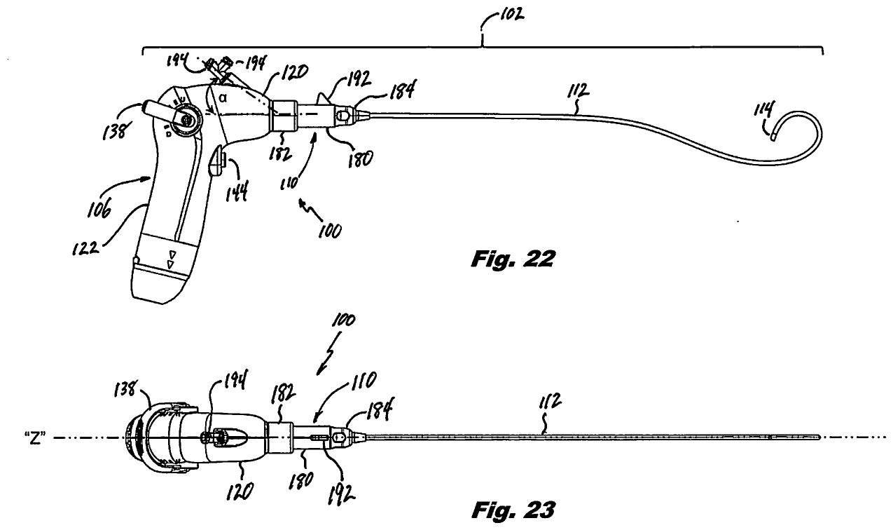

Referring now to FIGS. 21-24, another exemplary embodiment of a ureteroscope 100 according to the present disclosure is shown. In this exemplary embodiment, many of the components are designated by reference numerals discussed above and are the same as or similar to such like components and serve the same or similar functions as such like components. However, in this exemplary embodiment, the hub assembly 110 does not include the one or more luer ports 186, and there is no rotation drive system 150 as the hub assembly 110 generally remains fixed in position relative to the housing assembly 106 and the cannula 112. In this configuration, to change the orientation of the deflection plane “DP” of the cannula 112, the clinician rotates the handle 122 of the housing assembly 106 relative to the room. To provide the clinician with the location of the deflection plane (DP) of the cannula 112, the hub assembly 110 includes a sight member 192 positioned on the body 180 of the hub assembly 110 so that the sight member 192 is aligned with the deflection plane (DP) of the cannula 112. In this exemplary embodiment, one or more luer ports 194 are secured to a top of the body 120 of the housing assembly 106 so that part of the one or more luer ports 194 extend away from the body 120 at an angle “α” relative to the cannula axis “Z”, seen in FIG. 22. The one or more luer ports 194 are in communication with the channel 115 in the cannula 112 so that surgical instruments or fluid can be passed from the one or more luer ports 194, through the channel 115 in the cannula 112 and exit the distal part of the cannula. As a non-limiting example, the one or more luer ports 194 can be connected to channel 115 via a semi-rigid tube 189, seen in FIG. 24. In this exemplary embodiment, the one or more luer ports 194 remain fixed relative to the body 120 of the housing assembly 106.

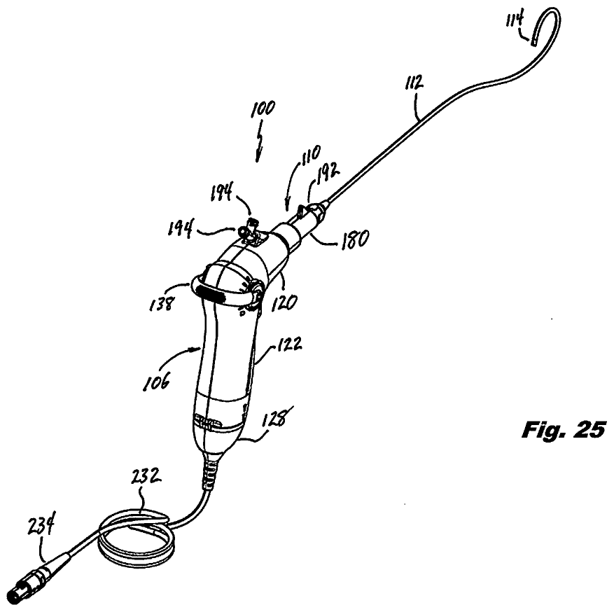

Referring now to FIGS. 25 and 26, another exemplary embodiment of an ureteroscope 100 according to the present disclosure is shown. In this exemplary embodiment, many of the components are designated by reference numerals discussed above and are the same as or similar to such like components and serve the same or similar functions as such like components. However, in this exemplary embodiment, the reusable portion 104 differs. More specifically, the reusable portion 104 communicates with the external processing/display unit 1000, seen in FIG. 11, via a cable connection. In this exemplary embodiment, cable 232 has a proximal part that includes one or more electrical contacts 236 passed through an opening in the cap 128 and are plugged into the charging connector 216 (FIG. 10) at the bottom end of the reusable portion 104. A distal part of the cable 232 includes a connector 234 that can be plugged into a suitable connector 1006 on the PD unit 1000. Operation of the ureteroscope 100 of FIGS. 25 and 26 is substantially the same as or similar to the embodiments of the endoscope described herein, except that data is exchanged with the PD unit 1000 via cable 232. The battery 204 of the reusable portion 104 can also be recharged via the cable 232.

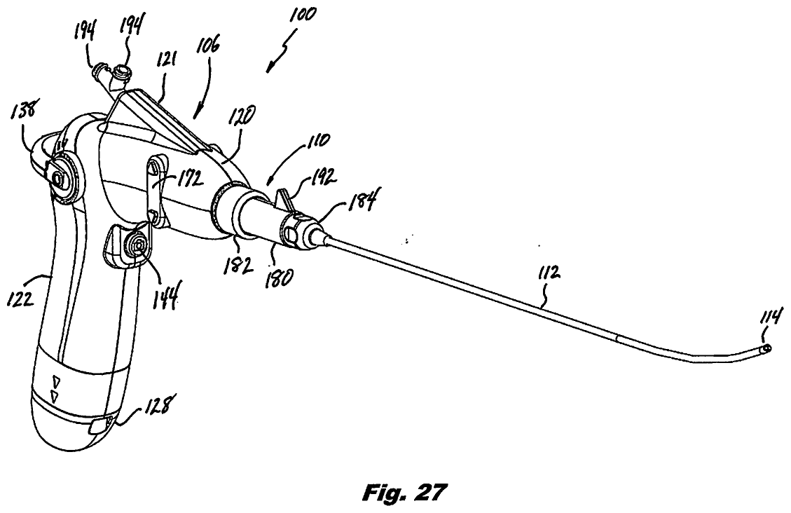

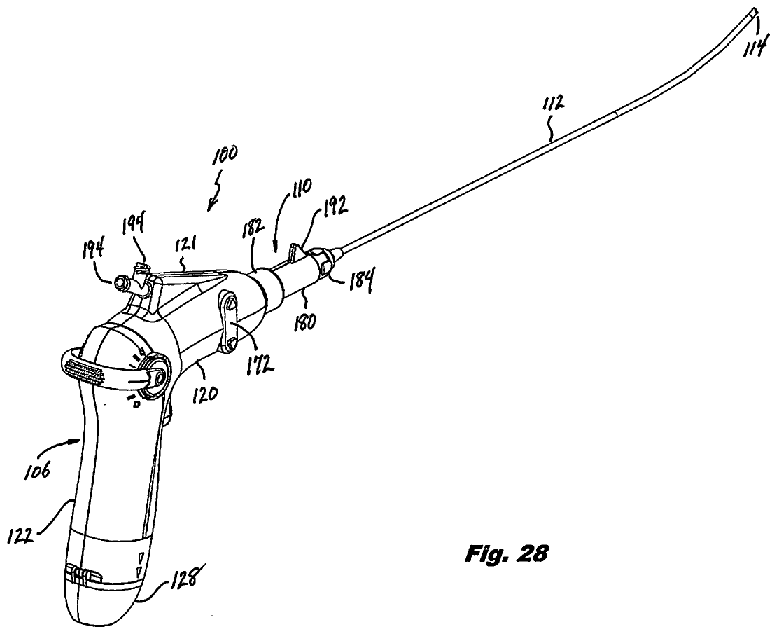

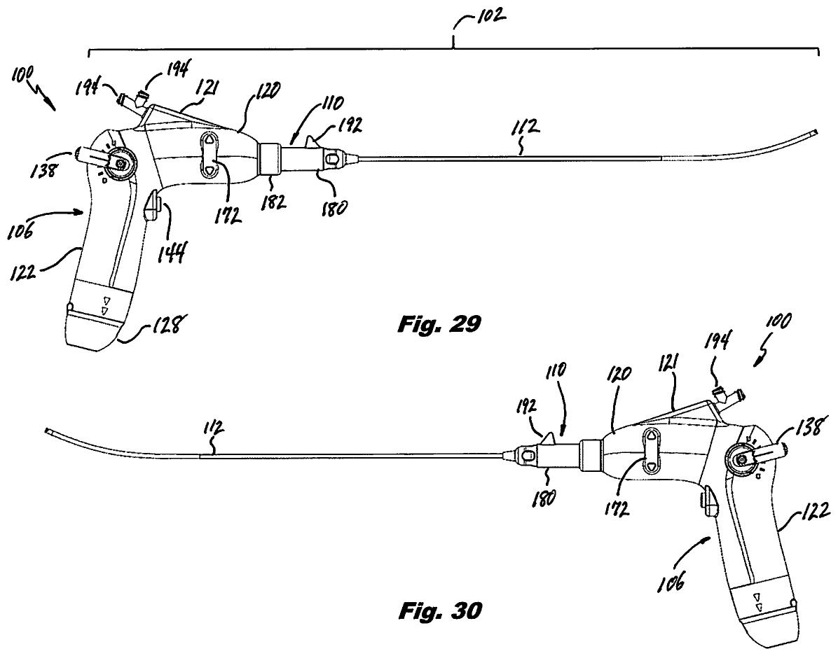

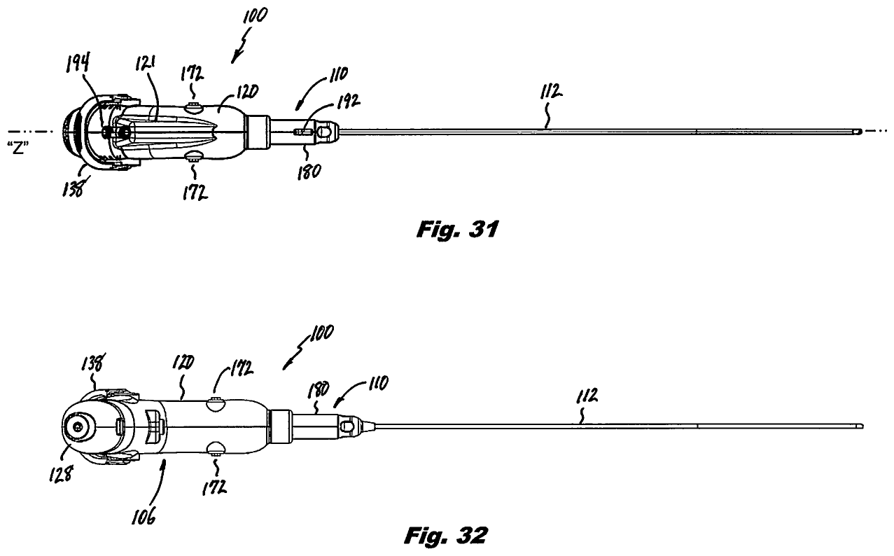

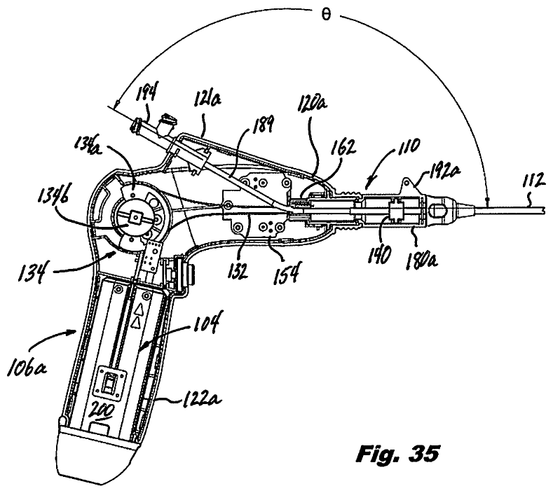

Referring now to FIGS. 27-36, another exemplary embodiment of a ureteroscope 100 according to the present disclosure is shown. In this exemplary embodiment, many of the components are designated by reference numerals discussed above and are the same as or similar to such like components and serve the same or similar functions as such like components. In this exemplary embodiment, the hub assembly 110 includes the sight member 192 described above. In addition, in this exemplary embodiment, the body 120 of the housing assembly 106 includes a port extension 121 defined by a first extension portion 121a of the first housing portion 200a and a second extension portion 121b of the second housing portion 200b that when joined form the port extension 121. The port extension 121 moves the one or more luer ports 194 proximally and raises the one or more luer ports 194 up and away from the lever 138 of the deflection control system 130, reducing possible interference with the operation of the lever 138 when surgical instruments are inserted into a luer port 194 or fluid is introduced into or aspirated from the luer ports 194. In the embodiment shown, the port extension 121 is configured so that at least one of the luer ports 194 is at a predefined angle “θ” relative to the cannula axis “Z” as shown in FIG. 35. As a non-limiting example, the predefined angle may be in the range of about 10 degrees to about 80 degrees. The one or more luer ports 194 are in communication with the channel 115 in the cannula 112 so that surgical instruments or fluid can be passed from the one or more luer ports 194, through the channel 115 in the cannula 112 and exit the distal part of the cannula. As a non-limiting example, the one or more luer ports 194 can be connected to the channel 115 via a semi-rigid tube 189, seen in FIG. 35. In this exemplary embodiment, the one or more luer ports 194 remain fixed relative to the port extension 121 of the housing assembly 106.

The exemplary embodiment of FIGS. 27-36, the rotation drive system 150 also includes a rocker switch 170 to control the operation of the motor 152 instead of the motor switches 164. The rocker switch 170 is electrically connected to the PCB 168 using internal wires (not shown). The rocker switch 170 has a rocker arm 172 that is pivotably mounted to the body 120 of the housing assembly 106 so that when the rocker arm 172 is pivoted in a first direction the motor 152 rotates in a first direction, and when the rocker arm 172 is pivoted in a second direction the motor 152 rotates in a second direction. For example, if the upper portion 172a of the rocker arm 172 is depressed, the motor 152 may rotate in a counter-clockwise direction, and if the lower portion 172b of the rocker arm 172 is depressed, the motor 152 may rotate in a clockwise direction.

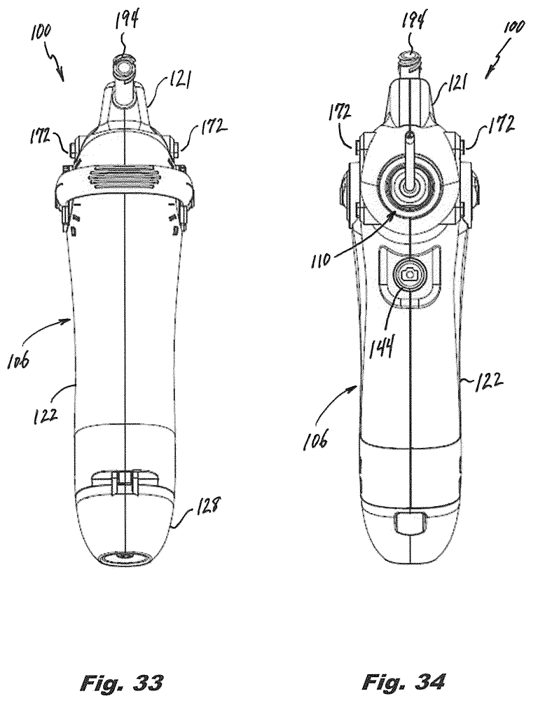

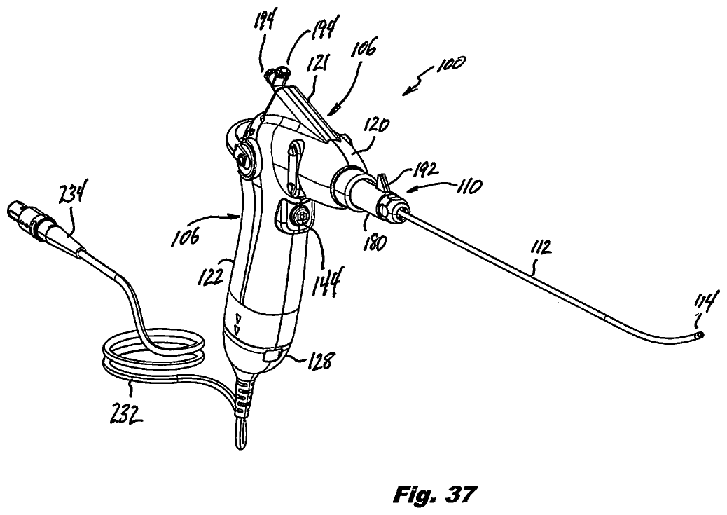

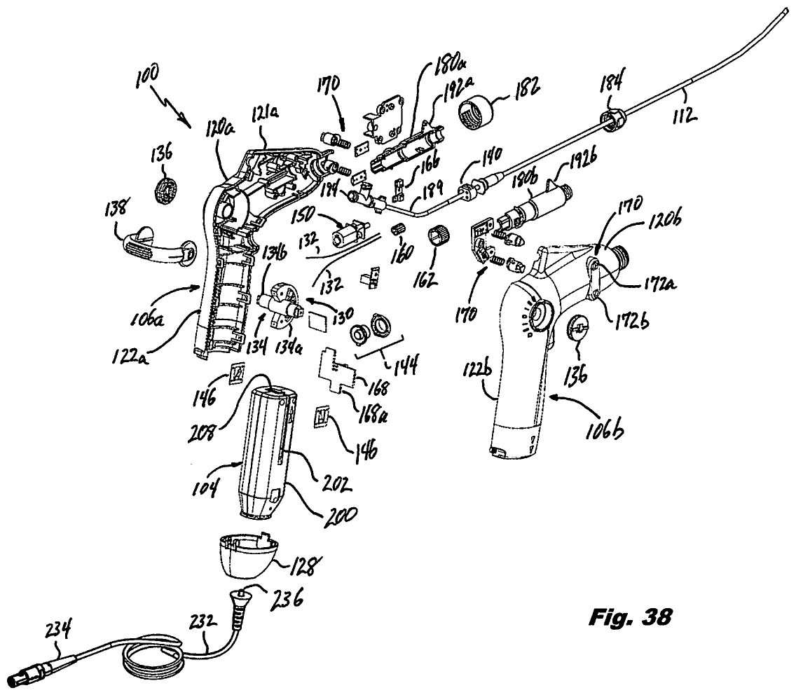

Referring now to FIGS. 37 and 38, another exemplary embodiment of an ureteroscope 100 according to the present disclosure is shown. In this exemplary embodiment, many of the components are designated by reference numerals discussed above and are the same as or similar to such like components and serve the same or similar functions as such like components. However, in this exemplary embodiment, the reusable portion 104 differs. More specifically, the reusable portion 104 communicates with the external processing/display unit 1000, seen in FIG. 11, via a cable connection. In this exemplary embodiment, cable 232 has a proximal part that includes one or more electrical contacts 236 passed through an opening in the cap 128 and are plugged into the charging connector 216 at the bottom end of the reusable portion 104. The distal part of the cable 232 includes a connector 234 that can be plugged into a suitable connector 1006 on the PD unit 1000. Operation of the ureteroscope 100 of FIGS. 33-36 is substantially the same as or similar to the embodiments of the endoscope described herein, except that data is exchanged with the PD unit 1000 via cable 232. The battery 204 of the reusable portion 104 can also be recharged via the cable 232.

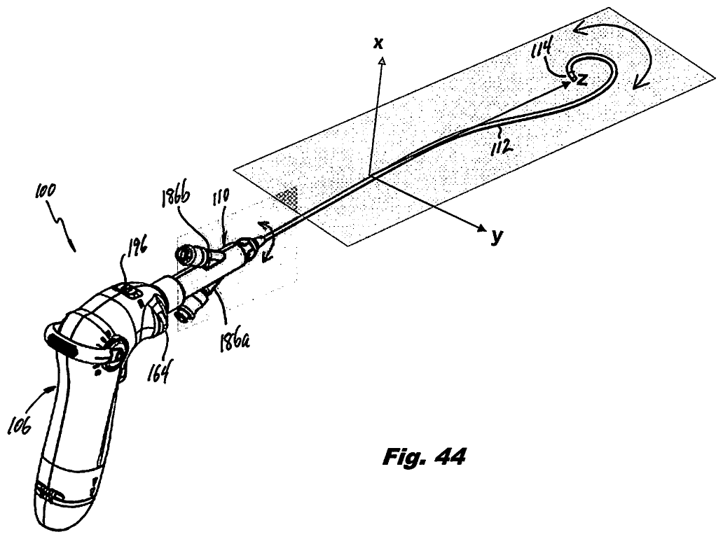

Turning now to FIGS. 39-50b, various operational features of the various embodiments of the ureteroscope 100 according to the present disclosure are shown and described. Endoscope 100 in this example is specially designed and useful as an ureteroscope although some embodiments thereof can be used in other medical procedures such as for male and female cystoscopy and for examining and treating the female reproductive system. The operational features of the ureteroscope 100 are described for kidney procedures. According to some embodiments, ureteroscope 100 can operate more accurately and efficiently than known ureteroscopes by enabling cannula rotation relative to a handle without requiring wrist or elbow rotation. Some embodiments enable particularly accurate and efficient positioning of a distal tip of a cannula tip relative to a target region or tissue in two types of rotation of the cannula relative to a handle-rotation of the cannula relative to the handle and rotation of the handle and thus the cannula relative to the patient and the room by hand motion. In some embodiments, the ureteroscope has a pistol-grip handle that enables a more natural and convenient way of holding the ureteroscope and controlling its functions. In some embodiments, the ureteroscope provides the clinician with an easy and unambiguous immediate indication that can be both visual and tactile of the current plane in which a curved distal part of a cannula is located. For the exemplary embodiments shown, there are two ports 186 that are aligned in a plane (the “luer plane” or “LP”) defined by a central axis of the luer ports. The two ports 186 are luer ports and for ease of description are designated with the identifiers 186a and 186b (FIG. 44). In addition, at least the tip portion of the distal part of the cannula 112 deflects in a plane (the “deflection plane” or “DP”) that includes the longitudinal axis “Z” of the cannula 112 that may also be referred to herein as the cannula axis “Z”. It is also noted that in some of the figures the cannula axis “Z” is aligned with the Z-axis of a three-dimensional cartesian coordinate system.

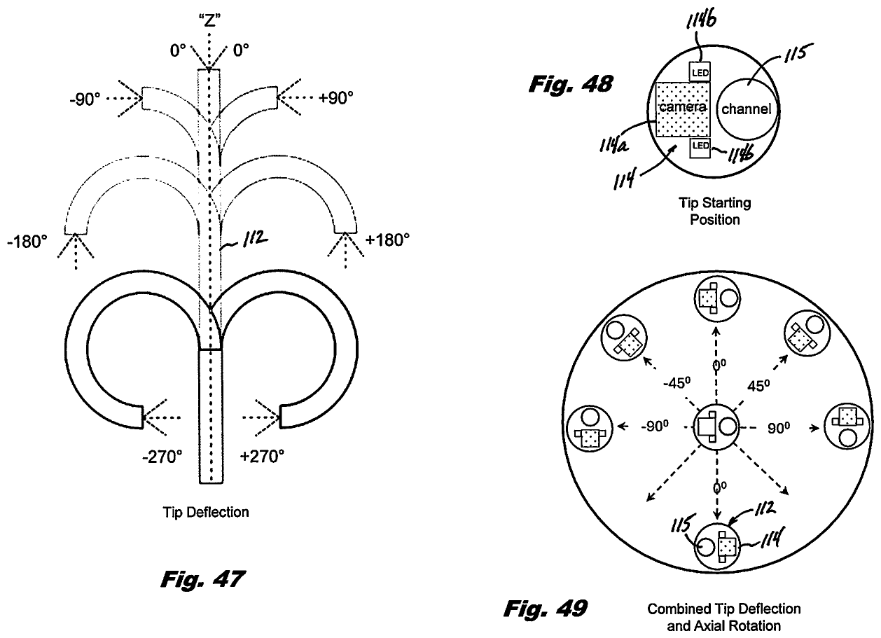

In the embodiment of FIGS. 39-41, the LP and DP are fixed relative to each other, and the resulting plane is referred to herein as the DP-LP plane. The cannula 112 can deflect in a positive or negative direction relative to the cannula axis “Z” in the DP-LP plane. For ease of description, the cannula axis “Z” is identified in FIGS. 39 and 47 as the zero-degree (0 degree) angle. FIG. 39 illustrates ureteroscope 100 in a starting position where the cannula 112 is deflected in a negative direction, e.g., upward in the X-Z plane, to an angle of about 270 degrees (−270 degrees). It is noted that the deflection of the cannula 112 is achieved by activating the deflection control system 130. For example, the deflection control system 130 can be manually activated using lever 138, seen in FIG. 39, or the deflection control system 130 can be manually activated using at least one of the deflection wheel levers 240 or 242, seen in FIG. 18. However, in this example the cannula deflection can be actuated using a motorized deflection control system 130 that can include a motor that is similar to the motor 152 coupled to the cable wheel 134 to which the cables 132 are attached.

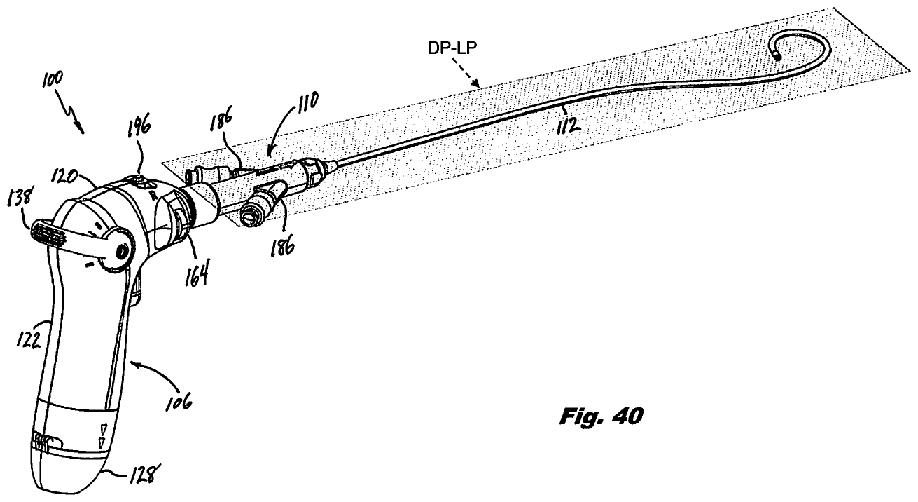

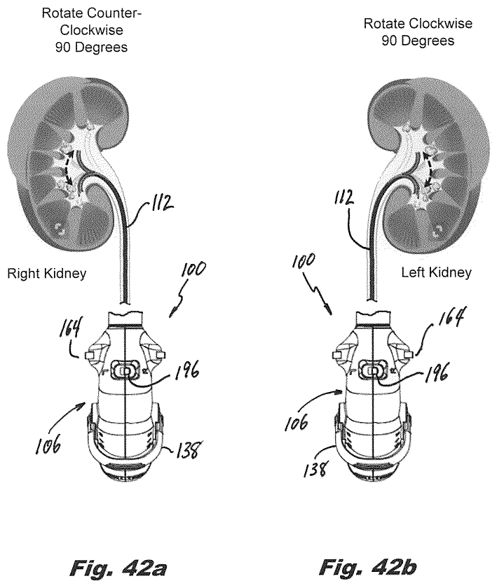

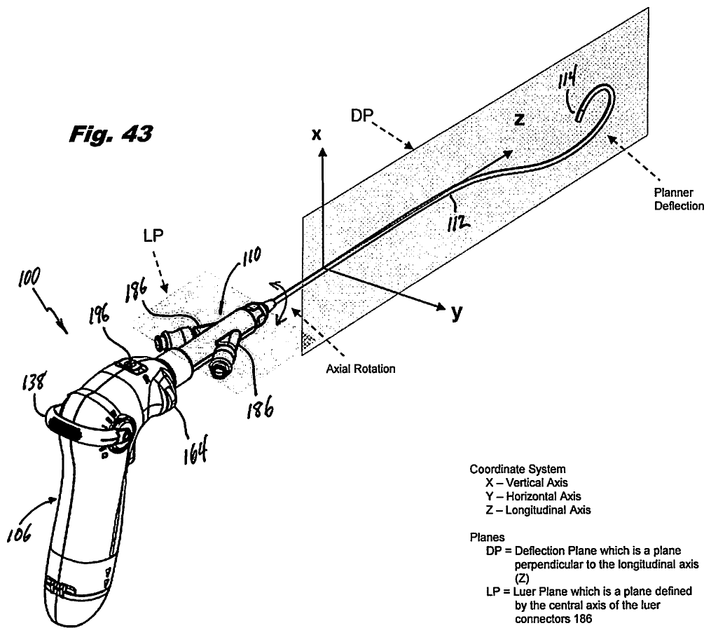

For a procedure on a patient's right kidney with the patient in the lithotomy position and with the ureteroscope 100 in the starting position of FIG. 39, the hub assembly 110 and cannula 112 can be rotated counter-clockwise (left) by moving the actuating arm 164a (FIG. 18) of the motor switch 164. In this example, the hub assembly 110 and cannula 112 are rotated about 90 degrees around the cannula axis “Z” to align the DP with the patient's right kidney, as shown in FIGS. 40 and 42a. With the DP aligned with the patient's right kidney, the luer port 186a that was oriented on the top relative to the housing assembly 106, e.g., at the 12:00 o'clock position looking distally, has rotated to the left side of the endoscope 100, e.g., at the 9:00 o'clock position looking distally, as shown in FIG. 40. With the DP aligned with the patient's right kidney, and after the distal end of cannula 112 has been angled relative to the Z axis as needed to reach the desired position at or in the right kidney, the deflection control system 130 can be actuated to deflect the tip portion of the cannula 112 to the desired position within the patient as shown in FIG. 42a. As noted, the tip portion of the cannula 112 can be deflected between +270 degrees and −270 degrees, seen in FIG. 47. With the DP aligned with the patient's right kidney, surgical instruments can be introduced into the luer ports 186 and thus to the target area within the patient's right kidney and/or fluid can be introduced into or aspirated from the luer ports 186 and thus to or from the target area within the patient's right kidney.

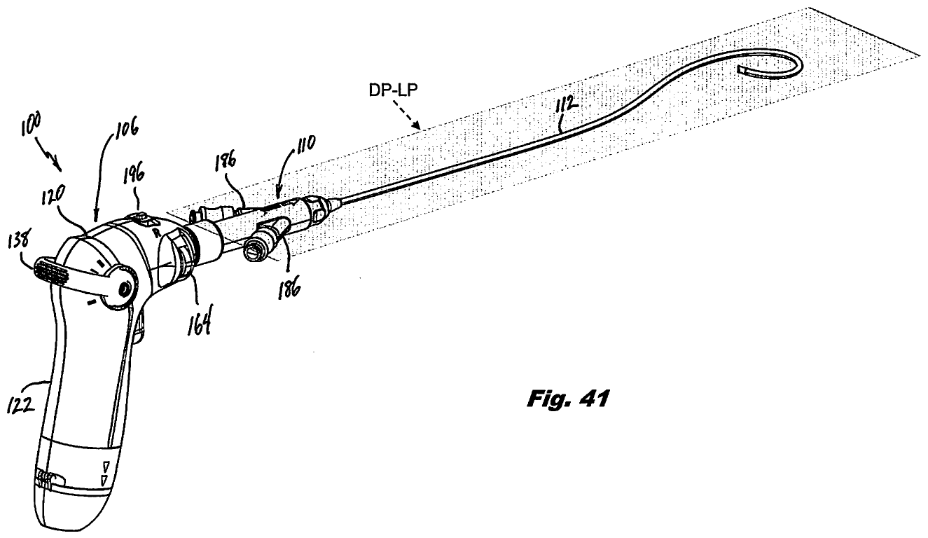

For a procedure on a patient's left kidney with the patient in the lithotomy position and with the ureteroscope 100 in the starting position of FIG. 39, the hub assembly 110 and cannula 112 can be rotated clockwise (right) by moving the actuating arm 164a of the motor switch 164. In this example, the hub assembly 110 and the cannula 112 are rotated about 90 degrees around the cannula axis “Z” to align the DP with the patient's left kidney, as shown in FIGS. 41 and 42b. With the DP aligned with the patient's left kidney, the luer port 186a that was oriented on the top relative to the housing assembly 106, e.g., at the 12:00 o'clock position looking distally, has rotated to the right side, e.g., at the 3:00 o'clock position looking distally, as shown in FIG. 41. With the DP aligned with the patient's left kidney, and after the distal end of cannula 112 has been angled relative to the Z axis as needed to reach the desired position at or in the left kidney the deflection control system 130 can be actuated by, for example, lever 138, to deflect the tip portion of the cannula 112 to the desired position within the patient as shown in FIG. 42b. As noted, the tip portion of the cannula 112 can be deflected between +270 degrees and −270 degrees, seen in FIG. 47. With the DP aligned with the patient's left kidney, surgical instruments can be introduced into the luer ports 186 and thus to the target area within the patient's right kidney and/or fluid can be introduced into or aspirated from the luer ports 186 and thus to or from the target area within the patient's left kidney.

It is noted, there are two kinds of rotations associated with the hub assembly 110 and the cannula 112. As described above, the first kind of rotation is Major Rotation (MAR) where the cannula 112 rotates with a large angle of rotation, e.g., a 22 degree, 45 degree, 90 degrees or 120 degree angle of rotation. As described above, the rotation drive system 150 can be a powered system that uses, for example, the motor 152 to achieve major rotation, or the rotation drive system 150 can be a manual system that uses, for example, thumbwheel 230, seen in FIG. 9, to achieve the Major Rotation. As described above, the second kind of rotation is Minor Rotation (MIR) where the cannula 112 rotates with smaller angles of rotation (subtle rotation), e.g., below a 22 degree angle of rotation. To achieve Major Rotation of the tip portion of the cannula, the clinician manually rotates the proximal part of hub assembly 110 or the cannula 110 relative to handle 122. The preferred method is to use MAR to align the DPs with the kidney space in the Y-Z horizontal plane and then use MIR to move the distal tip slightly in the X direction inside the kidney space. Minor rotation can be done during the medical procedure by grasping both handle 122 and hub 110 and rotating then around the cannula axis relative to each other, or by simply rotating the handle relative to the patient and the room.

In the embodiment of FIGS. 43-45, the LP and DP are orthogonal relative to each other. The cannula 112 can deflect in a positive or negative direction relative to the cannula axis “Z” in the DP. For ease of description, cannula axis “Z” is identified as the zero-degree (0 degree) angle, seen in FIGS. 39 and 47. FIG. 43 illustrates the ureteroscope 100 in a starting position where the cannula 112 is deflected in a negative direction, e.g., upward in the X-Z plane, to an angle of about 270 degrees (−270 degrees). It is noted that the deflection of the cannula 112 is achieved by activating the deflection control system 130. For example, the deflection control system 130 can be manually activated using lever 138, seen in FIG. 43, or the deflection control system 130 can be manually activated using at least one of the deflection wheel levers 240 or 242, seen in FIG. 18. However, the cannula deflection can be actuated using a motorized deflection control system 130 that can be a motor that is similar to the motor 152 coupled to the cable wheel 134 to which the cables 132 are attached.

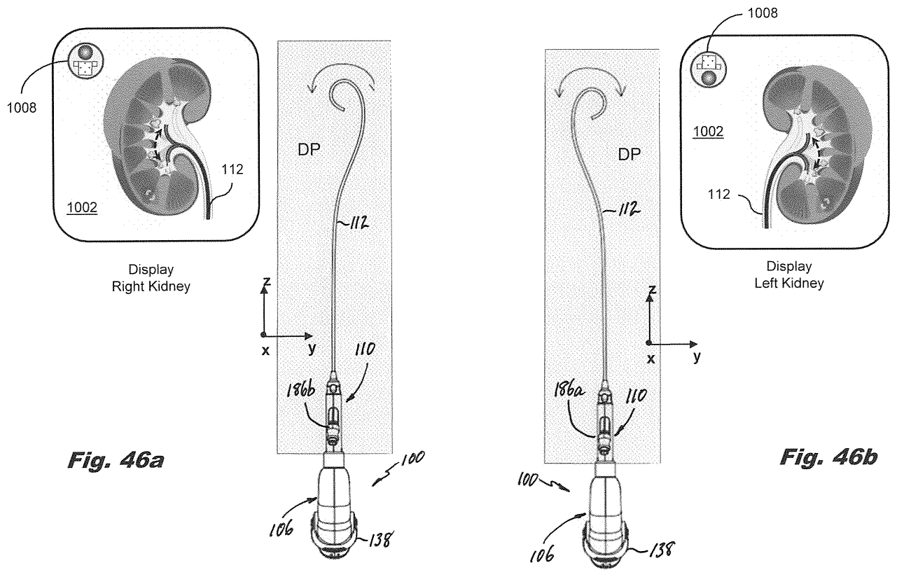

For a procedure on a patient's right kidney with the patient in the lithotomy position and with the ureteroscope 100 in the starting position of FIG. 43, the hub assembly 110 and cannula 112 can be rotated counter-clockwise (left) by moving the actuating arm 164a of the motor switch 164. In this example, the hub assembly 110 and cannula 112 are rotated about 90 degrees around the cannula axis “Z” to align the DP with the patient's right kidney, as shown in FIGS. 44 and 46a. With the DP aligned with the patient's right kidney, the luer port 186b that was oriented on the right side relative to the housing assembly 106, e.g., at the 3:00 o'clock position looking distally, has rotated to the top of the ureteroscope 100, e.g., at the 12:00 o'clock position looking distally, as shown in FIG. 44. In addition, the luer port 186a that was oriented on the left side relative to the housing assembly 106, e.g., at the 9:00 o'clock position looking distally, has rotated to the bottom of the ureteroscope 100, e.g., at the 6:00 o'clock position looking distally, as shown in FIG. 44. With the DP aligned with the patient's right kidney, the deflection control system can be actuated to deflect the tip portion of the cannula 112 to the desired position within the patient as shown in FIG. 46a. As noted, the tip portion of the cannula 112 can be deflected between +270 degrees and −270 degrees, seen in FIG. 47. With the DP aligned with the patient's right kidney and the luer ports 186 oriented in the top and bottom positions described above, surgical instruments can be introduced into luer port 186b and thus into the target area within the patient's right kidney, and fluid can be introduced into or aspirated from the luer port 186a and thus into or out of the target area within the patient's right kidney.

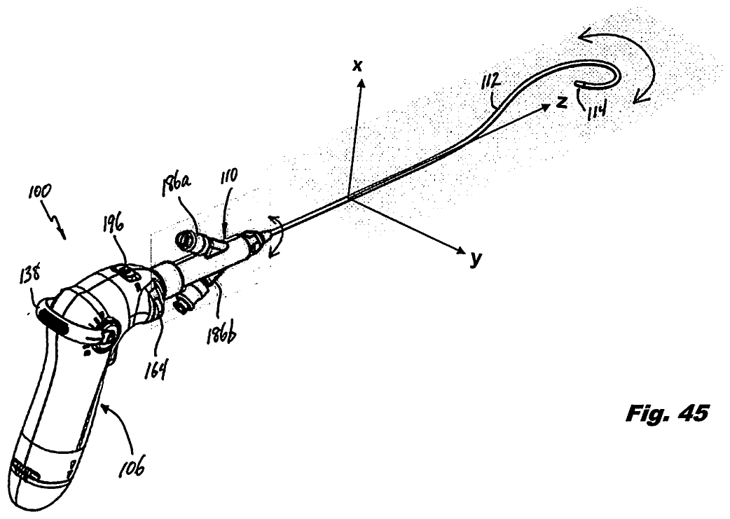

For a procedure on a patient's left kidney with the patient in the lithotomy position and with the ureteroscope 100 in the starting position, seen in FIG. 43, the hub assembly 110 and cannula 112 can be rotated clockwise (right) by moving the actuating arm 164a of the motor switch 164. In this example, the hub assembly 110 and the cannula 112 are rotated about 90 degrees around the cannula axis “Z” to align the DP with the patient's left kidney, as shown in FIGS. 45 and 46b. With the DP aligned with the patient's left kidney, the luer port 186a that was oriented on the left side relative to the housing assembly 106, e.g., at the 9:00 o'clock position looking distally, has rotated to the top of the ureteroscope 100, e.g., at the 12:00 o'clock position looking distally, as shown in FIG. 45. In addition, the luer port 186b that was oriented on the right side relative to the housing assembly 106, e.g., at the 3:00 o'clock position looking distally, has rotated to the bottom of the ureteroscope 100, e.g., at the 6:00 o'clock position looking distally, as shown in FIG. 45. With the DP aligned with the patient's left kidney, the deflection control system 130 can be actuated to deflect the tip portion of the cannula 112 to the desired position within the patient, as shown in FIGS. 45 and 46b. As noted, the tip portion of the cannula 112 can be deflected between +270 degrees and −270 degrees, seen in FIG. 47. With the DP aligned with the patient's left kidney and the luer ports 186 oriented in the top and bottom positions described above, preferably surgical instruments can be introduced into luer port 186a and thus into the target area within the patient's left kidney, and fluid can be introduced into or aspirated from the luer port 186b and thus into or out of the target area within the patient's left kidney.

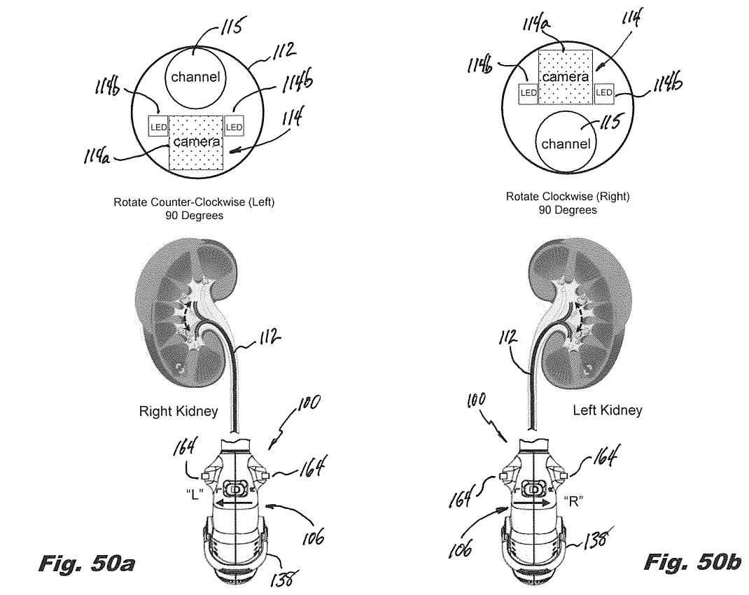

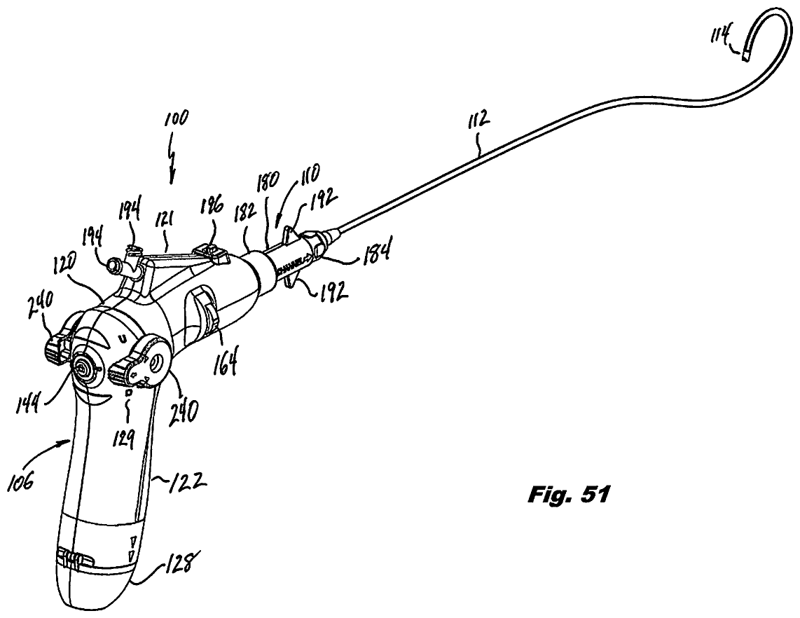

Referring now to FIGS. 50a-50b, an exemplary embodiment of an image orientation adjustment process is shown and described in relation to the insertion of a cannula 112 of the ureteroscope 100 of FIGS. 43-45 into a ureter of a patient. When the cannula 112 is staged for insertion, the ureteroscope 100 is in the starting position shown in FIG. 43. In this starting position, the tip portion of cannula 112, looking proximally. is shown in FIG. 48, where the channel 115 in the cannula is oriented on the right side of the tip portion and the imaging module 114 is oriented on the left side of the tip portion. As noted, the imaging module 114 includes a camera 114a and one or more illumination sources, here LEDs 114b. When the cannula 112 is rotated as described herein, the one or more channels 115 and the imaging module 114 rotate with the cannula 112. FIG. 49 shows a tip deflection and cannula axial rotation chart that illustrates the orientation of the imaging module 114 at various angles of rotation of the cannula 112.

For a procedure on a patient's right kidney with the patient in the lithotomy position, the cannula 112 in the starting position is inserted into the patient's right ureter, as shown in FIG. 50a. As the tip portion of the cannula 112 enters the kidney, the hub assembly 110 and cannula 112 can be rotated counter-clockwise 90 degrees by moving the actuating arm 164a of the motor switch 164 to align the DP with the right kidney. Rotating the hub assembly 110 and cannula 112 by 90 degrees (−90 degrees) rotates the channel 115 and imaging module 114 by 90 degrees (−90 degrees), as shown in FIG. 50a. Rotating the imaging module 114 by 90 degrees (−90 degrees) also rotates the image to be displayed on display 1002 of the PD unit 1000 by 90 degrees (−90 degrees). In order to present the image to the clinician in a way as if the cannula 112 had not been rotated, e.g., to present the kidney in an upright orientation, an image control switch 196 is provided within the housing assembly 106. The image control switch 196 is electrically connected to the PCB 168 and the electrical contacts 168a of the printed PCB 168 are electrically connected to the one or more electrical contacts 208 within the reusable portion 104. The image adjustment can be performed by the control and processing electronics 210 within the reusable portion 104 or by the processing/display unit 1000. Activation of the image control switch 196 in the direction “L” in FIG. 50a informs the control and processing electronics 210 or the processing/display unit 1000 that the ureteroscope 100 is pre-rotate 90 degrees (−90 degrees) axially. The control and processing electronics 210 or the processing/display unit 1000 then performs an autocorrect function so that the image of the right kidney is displayed in an upright manner as shown in FIGS. 46a and 50a. Similarly, activation of the image control switch 196 in the direction “R” in FIG. 50b informs the control and processing electronics 210 or the processing/display unit 1000 that the ureteroscope 100 is pre-rotate 90 degrees (+90 degrees) axially. The control and processing electronics 210 or the processing/display unit 1000 then performs an autocorrect function so that the image of the left kidney is displayed in an upright manner as shown in FIGS. 46b and 50b.

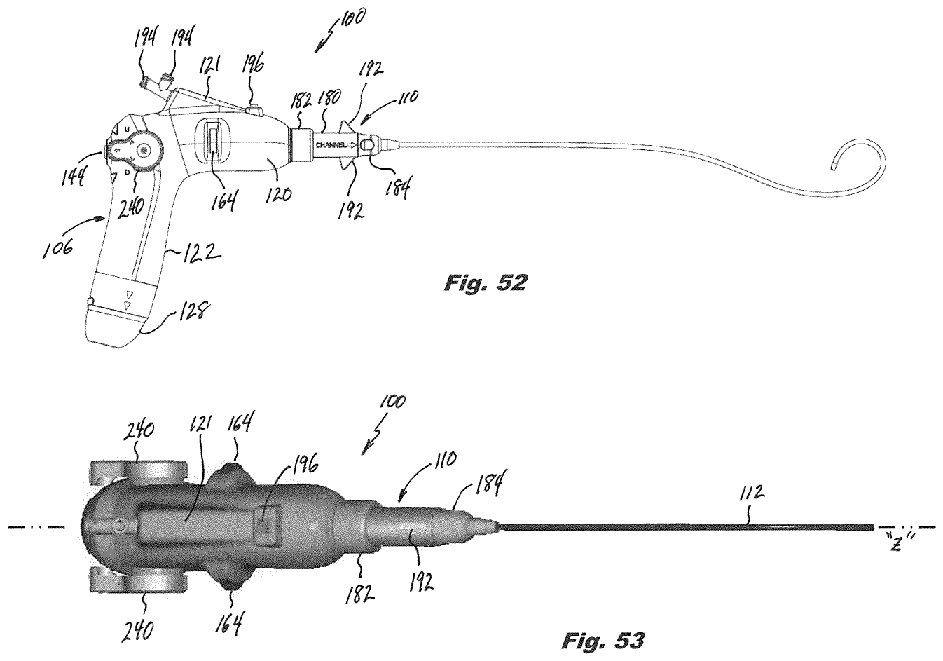

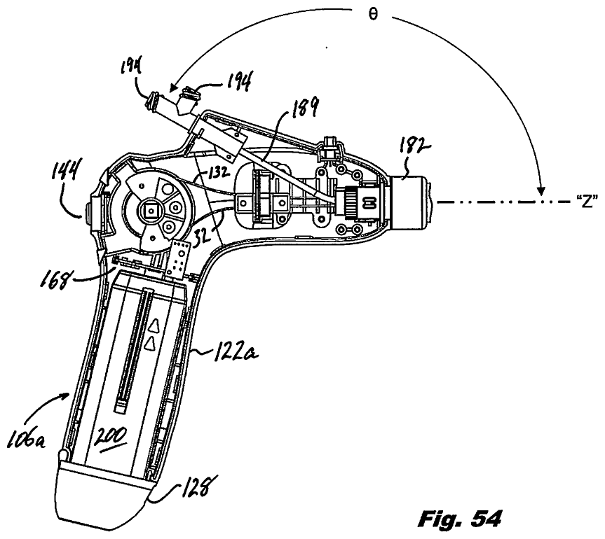

Referring now to FIGS. 51-56, another exemplary embodiment of a ureteroscope 100 according to the present disclosure is shown. In this exemplary embodiment, many of the components are designated by reference numerals discussed above and are the same as or similar to such like components and serve the same or similar functions as such like components. However, in this exemplary embodiment, the deflection wheel levers 240 of the deflection control system 130 differ. In addition, the body 120 of the housing assembly 106 includes the port extension 121, as described herein. The switch 144, seen in FIG. 51, is moved to the proximal end of the housing assembly 106 and is accessible from an exterior of the housing assembly 106. Further, multiple sight members 192 can be associated with the body 180 of the hub assembly 110. In addition, the reusable portion 104 is configured to better dissipate heat generated by PCB 168 during operation of the ureteroscope 100 as described below.

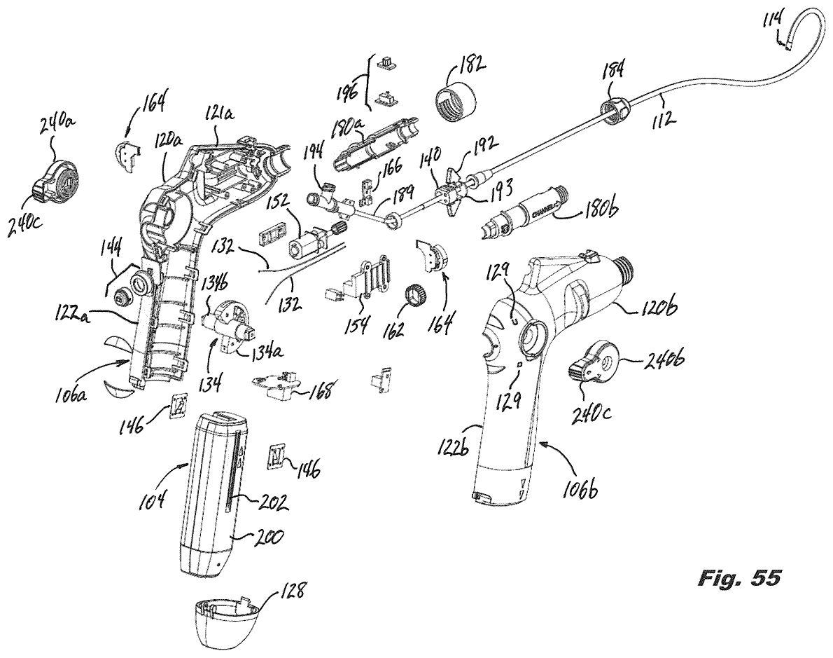

In this exemplary embodiment, the deflection control system 130 includes a pair of deflection wheel levers 240 attached to arms 134b of the cable wheel 134. More specifically, a left deflection wheel lever 240a is attached to the end of the arm 134b passing through the aperture 124 in the left housing cover 106a, and a right deflection wheel lever 240b is attached to the other end of the arm 134b passing through the aperture 124 in the right housing cover 106b, for example. The left and right deflection wheel levers 240 are rotatable relative to the housing assembly 106 such that clockwise and counter-clockwise rotation of the left and/or right deflection wheel levers 240 cause the cannula 112 and thus the imaging module 114 to deflect in the deflection plane (DP), seen in FIG. 39, between the first full deflection position and the second full deflection position described above. It is noted that the deflection wheel levers 240 have one rib 240c extending therefrom, as shown in FIG. 55. However, as described herein, the deflection wheel levers 240 may have multiple ribs 240c. The rib or ribs 240c facilitate easy rotation of the deflection wheel levers 240 by a clinician. It is also noted that the body 120 and/or handle 122 of the housing assembly 106 may include one or more indicia 129 that is indicative of the direction of deflection of the distal portion of the cannula 112 in the deflection plane (DP). In this embodiment, the one or more indicia 129 include two structural marks “U” and “D” representing a deflection of the distal portion of the cannula 112 in an upward direction in the deflection plane (DP) and a downward direction in the deflection plane (DP), respectively.

Continuing to refer to FIGS. 51-55, the port extension 121 is defined by the first extension portion 121a, seen in FIG. 55, of the first housing portion 200a and the second extension portion 121b of the second housing portion 200b that when joined form the port extension 121, seen in FIG. 51. The port extension 121 moves the one or more luer ports 194 proximally and raises the one or more luer ports 194 up and away from the body 120 and thus from the deflection wheel levers 240 of the deflection control system 130, reducing possible interference with the operation of the deflection wheel levers 240 when surgical instruments are inserted into a luer port 194 or fluid is introduced into or aspirated from the luer ports 194. In the embodiment shown, and as described herein, the port extension 121 is configured so that at least one of the luer ports 194 is at a predefined angle “θ” relative to the cannula axis “Z” as shown in FIGS. 54. As a non-limiting example, the predefined angle may be in the range of about 10 degrees to about 80 degrees. The one or more luer ports 194 are in communication with the channel 115 in the cannula 112 so that surgical instruments or fluid can be passed from the one or more luer ports 194, through the channel 115 in the cannula 112 and exit the distal part of the cannula. As a non-limiting example, the one or more luer ports 194 can be connected to the channel 115, seen in FIG. 48, via a semi-rigid tube 189, seen in FIGS. 54 and 55. In this embodiment, the one or more luer ports 194 remain fixed relative to the port extension 121 of the housing assembly 106.

As described above, in instances where a photograph or video of the target area within a patient is desired, a photo switch 144 within the housing assembly 106 of the single-use portion 102 and accessible from an exterior of the housing assembly 106 may be used. In this embodiment, the photo switch 144, seen in FIG. 51, is moved to the proximal end of the housing assembly 106 and is accessible from an exterior of the housing assembly 106. The photo switch 144 may be electrically connected to the imaging module 114 via internal wires (not shown) in the single-use portion 102, so that when the clinician activates the photo switch 144, the camera 114a of the imaging module 114 snaps a photo of the target area.

Continuing to refer to FIGS. 51-55, to provide the clinician with the location of the deflection plane (DP) of the cannula 112, seen in FIG. 43, the hub assembly 110 includes multiple sight members 192 extending from the body 180 of the hub assembly 110 so that the sight members 192 are aligned with the deflection plane (DP) of the cannula 112. In this embodiment, the sight members 192 may be color coded so that a first color, e.g., red and green or black and white, may be indicative of deflection of the distal portion of the cannula 112 in a first direction in the deflection plane (DP), and a second color, e.g., red and green, may be indicative of deflection of the distal portion of the cannula 112 in a second direction in the deflection plane (DP). Further, the first color may be associated with, for example, the “U” indicia 129, seen in FIG. 51, representing a deflection of the distal portion of the cannula 112 in the upward direction corresponding to the first color sight member 192, and the “D” the indicia 129 representing a deflection of the distal portion of the cannula 112 in the downward direction corresponding to the second color sight member 192. Alternatively, a single sight member 192 may be used, extending up in the illustrated view, an colored in a contrasting color such as black.

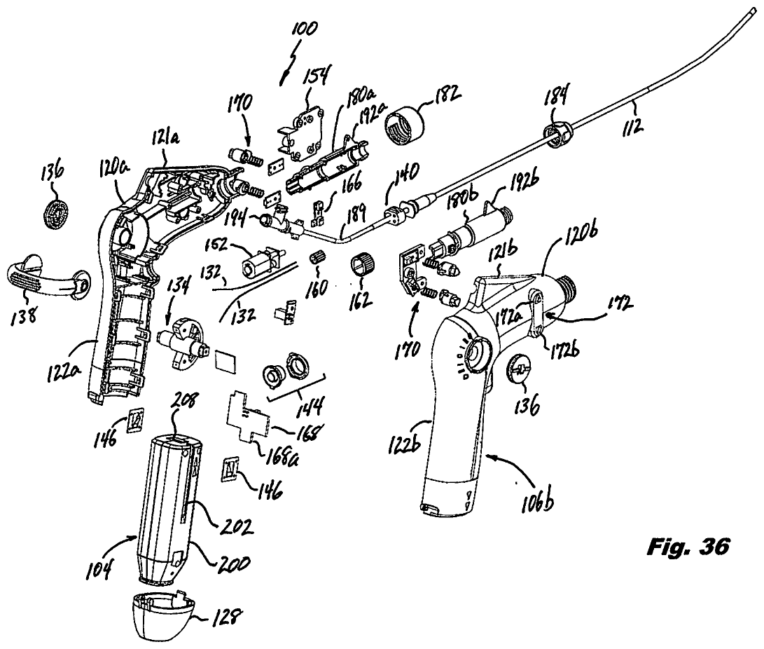

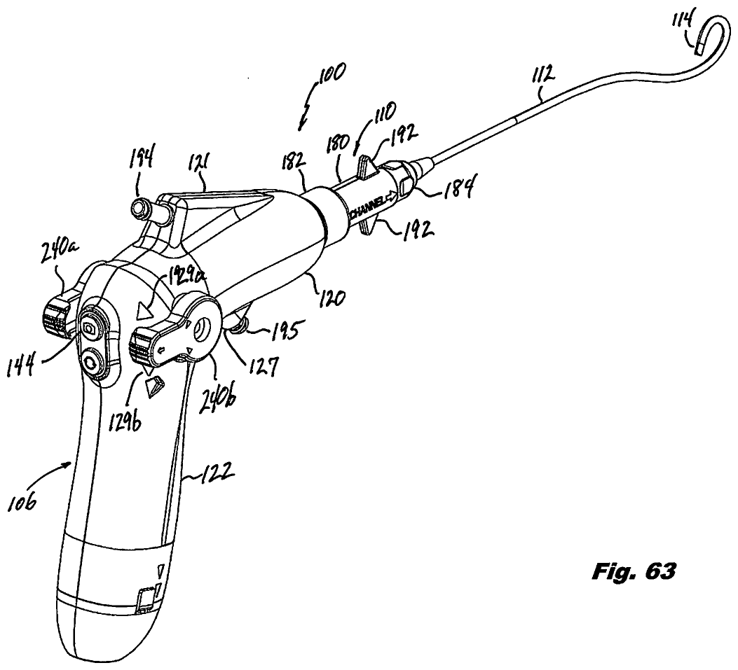

Further, as the hub assembly 110 is rotated relative to handle 122 by, for example, the rotation drive system 150, the sight members 192 rotate providing the clinician a visual indication of the deflection plane (DP) of the cannula 112, similar to that shown in FIG. 63. In the exemplary embodiment shown in FIG. 55, the sight members 192 are integral with or monolithically formed into a hub 193 that may be part of the cable housing 140 or a separate member that is positioned within the body 180. In another exemplary embodiment, the sight members 192 may be formed by a left sight member portion 192a and a right sight member 192b that may be integral with or monolithically formed into the left body cover 180a and a right body cover 180b, similar to that shown in FIG. 36, so that when the left body cover 180a and a right body cover 180b are joined to form the body 180, the sight members 192 are formed.

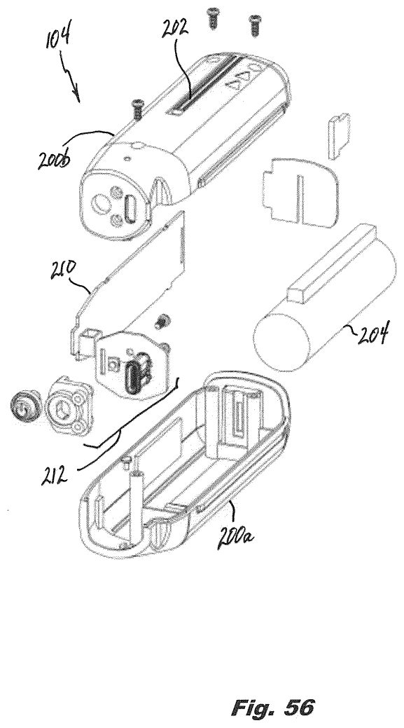

Referring again to FIG. 56, another exemplary embodiment of the reusable portion 104 is shown. In this exemplary embodiment, the housing 200a,b of the reusable portion 104 is configured to better dissipate heat generated by the rechargeable battery 204 and/or the control and processing electronics 210 within the reusable portion 104 during operation of the ureteroscope 100. In this exemplary embodiment, the first housing portion 200a and the second housing portion 200b of the housing 200 can be made of different materials, where at least one of the housing portions 200a and 200b may be made of a thermally conductive material that transfers heat from within the hollow portion of the housing 200 to the exterior of the housing 200. For example, the first housing portion 200a may be made of a plastic material and the second housing portion 200b may be made of a metallic material. The plastic material may be a thermoplastic material. Non-limiting examples of thermoplastic materials include polycarbonate (PC), and a combination of polycarbonate (PC) and acrylonitrile-butadiene-styrene terpolymer (ABS). Non-limiting examples of the metallic material include aluminum, aluminum nitride, copper, gold and silver. In this exemplary embodiment, the first housing portion 200a and a second housing portion 200b can be joined by, for example, adhesives or mechanical fasteners. A non-limiting example of mechanical fasteners includes snap-fit connections. In another exemplary embodiment, the first housing portion 200a and the second housing portion 200b of the housing 200 can be made with one or more openings, e.g., slots, (not shown) configured to permit heat generated within the hollow portion of the housing 200 to escape the housing.

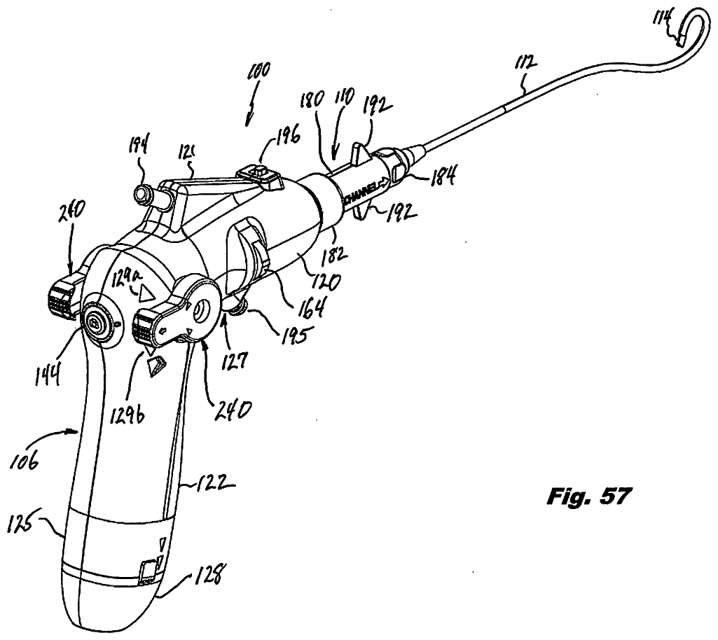

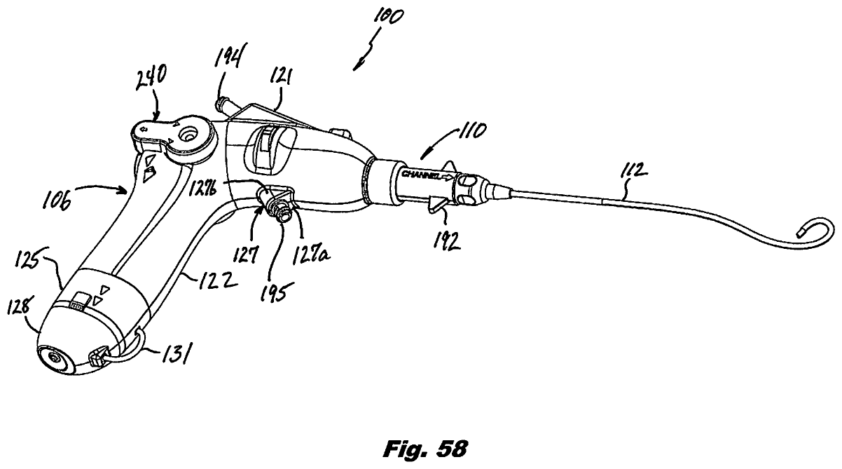

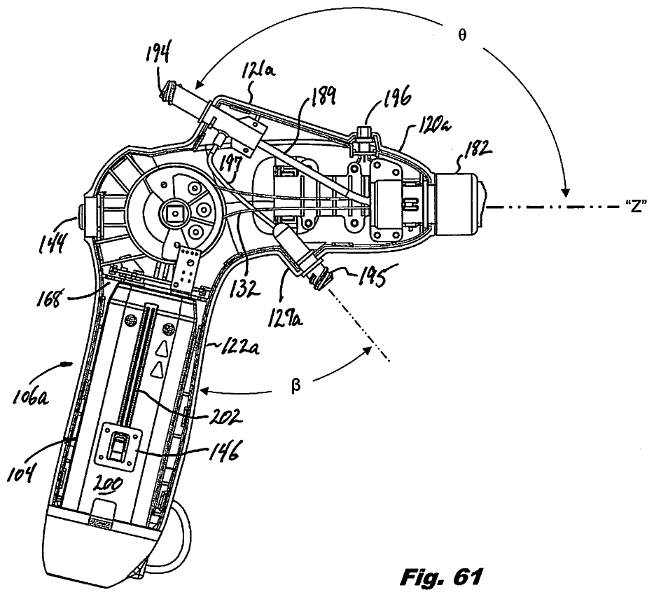

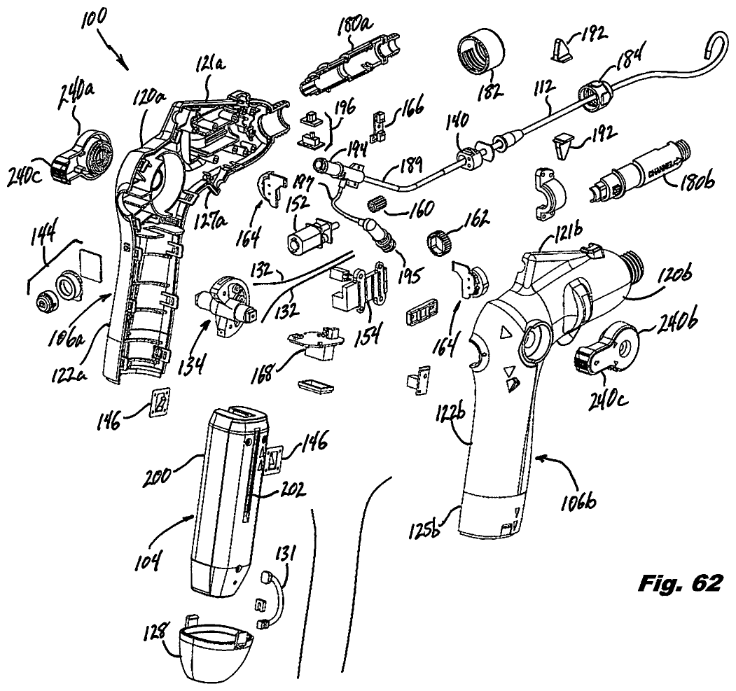

Referring now to FIGS. 57-62, another exemplary embodiment of a ureteroscope 100 according to the present disclosure is shown. In this exemplary embodiment, many of the components are designated by reference numerals discussed above and are the same as or similar to such like components and serve the same or similar functions as such like components. However, in this exemplary embodiment, the body 120 of the housing assembly 106 includes a second port extension 127 defined by a first extension portion 127a of the first housing portion 200a and a second extension portion 127b of the second housing portion 200b that when joined form the port extension 127. The second port extension 127 includes one or more luer ports 195 extending from a bottom side of the housing 200, as shown in FIG. 58. The one or more luer ports 195 of the second port extension 127 may be in communication, e.g., fluid communication, with the one or more luer ports 194 of the port extension 121 using, for example, a semi-rigid tube 197, shown in FIG. 61. In another exemplary embodiment, the one or more luer ports 195 of the second port extension 127 may be in communication, e.g., fluid communication, with the semi-rigid tube 189 connecting the one or more luer ports 194 to the channel 115 of the cannula 112 via the semi-rigid tube 197. In this exemplary embodiment, the one or more luer ports 194 remain fixed relative to the port extension 121 of the housing assembly 106, and the one or more luer ports 195 remain fixed relative to the port extension 127 of the housing assembly 106.

Preferably, the one or more luer ports 194 and 195 shown in FIGS. 57-62, include a single luer port 194 extending from the port extension 121 and a single luer port 195 extending from the second port extension 127, as shown. In this exemplary configuration, the luer port 194 extending from the port extension 121 may be used as a device port where surgical instruments and other devices may be inserted into the luer port 194 and passed through the cannula 112 to exit the distal portion (or end) of the cannula 112. In addition, the luer port 195 extending from the second port extension 127 may be used as a fluid port where fluid may be introduced into luer port 195, and passed through the channel 115 to exit the distal end of the cannula 112, or fluid at the surgical site may be aspirated from the surgical site through the channel 115 of the cannula 112 to exit the luer port 195 to a suitable container. In the embodiment shown in FIGS. 57-62, the port extension 121 is configured so that the luer port 194 is at a predefined angle “θ” relative to the cannula axis “Z” as shown in FIG. 61. As a non-limiting example, the predefined angle may be in the range of about 10 degrees to about 80 degrees. The port extension 127 is configured so that the luer port 195 is at a predefined angle “β” relative to the handle 122 of the housing assembly 106, as shown in FIG. 61. As a non-limiting example, the predefined angle may be in the range of about 10 degrees and about 80 degrees.

As described above, the one or more luer ports 194 of the port extension 121 moves the luer ports 194 proximally and raises the one or more luer ports 194 of the port extension 121 up and away from the lever 240 of the deflection control system 130, reducing possible interference with the operation of the lever 240 when surgical instruments are inserted into the luer port 194 or fluid is introduced into or aspirated from the luer port 194. Further, having the one or more luer ports 195 at the bottom of the housing assembly 106 and angled away from the handle 122 toward the distal direction, permits accidental fluid leaks at the luer port 195 to flow away from the handle 122.

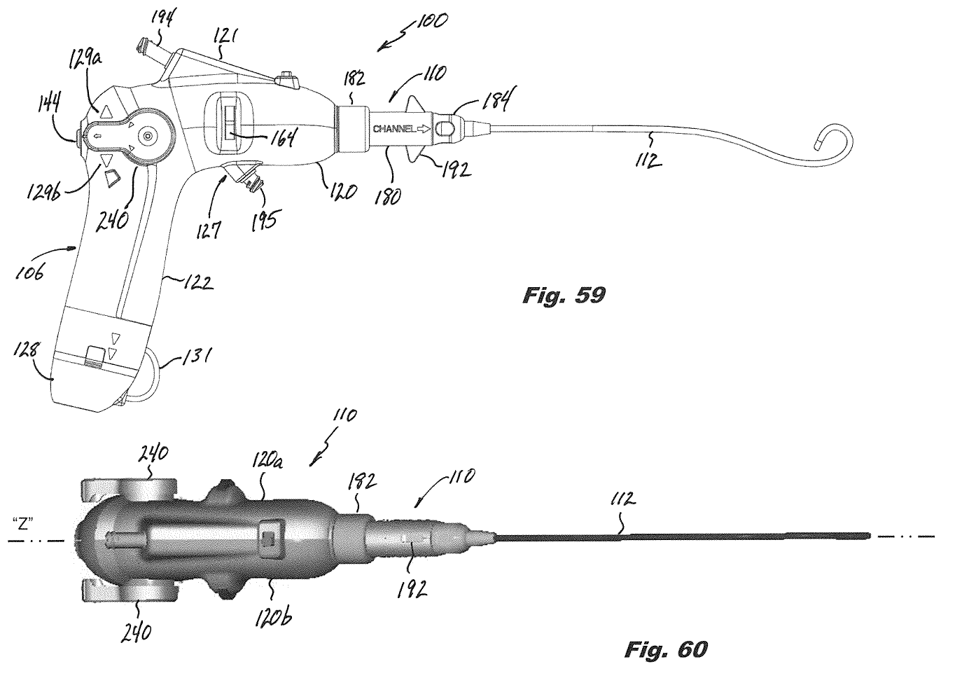

In this embodiment, the sight members 192 may also be color coded so that a first color, e.g., red, may be indicative of deflection of the distal portion of the cannula 112 in a first direction in the deflection plane (DP), and a second color, e.g., green, may be indicative of deflection of the distal portion of the cannula 112 in a second direction in the deflection plane (DP). Alternatively, only a single sight member can be used, pointing up. It is also noted that the body 120 and/or handle 122 of the housing assembly 106 may include the one or more structural indicia 129 that may also be indicative of the direction of deflection of the distal portion of the cannula 112 in the deflection plane (DP). As a non-limiting example, the one or more indicia 129 may include a first pointer 129a and a second pointer 129b. Further, the first color may be associated with, for example, the first pointer 129a, seen in FIG. 59, representing a deflection of the distal portion of the cannula 112 in the first direction corresponding to the first color sight member 192, and the second color may be associated with, for example, the second pointer 129b, seen in FIG. 59, representing a deflection of the distal portion of the cannula 112 in the second direction corresponding to the second color sight member 192.

In embodiment described herein, the cap 128 may be secured to the distal part 125 of the handle 122 by a hinge, e.g., a living hinge or a mechanical hinge, such that the cap 128 is pivotable between an open position permitting access to the open distal part 125 of the handle 122 and a closed position preventing access to the open distal part 125 of the handle 122. In another exemplary embodiment, the cap 128 may be secured to the distal part 125 of the handle 122 by a tether 131, such that the cap 128 is movable between an open position permitting access to the open distal part 125 of the handle 122 and a closed position preventing access to the open distal part 125 of the handle 122. The cap 128 may be removably secured to the distal part 125 of the handle 122 by a snap-fit connection.

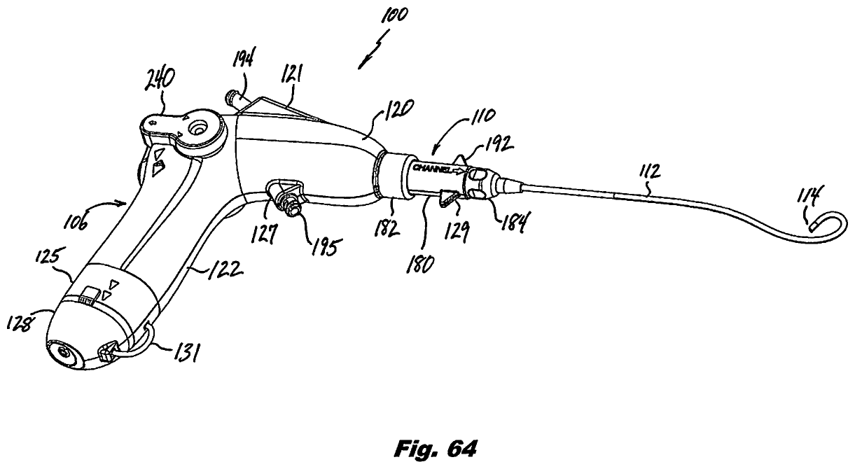

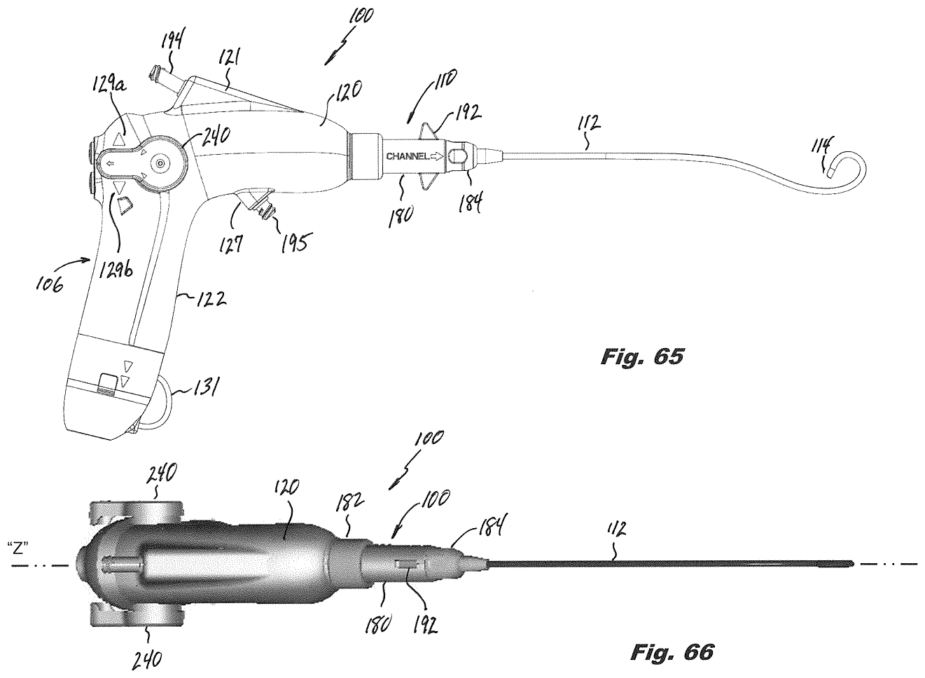

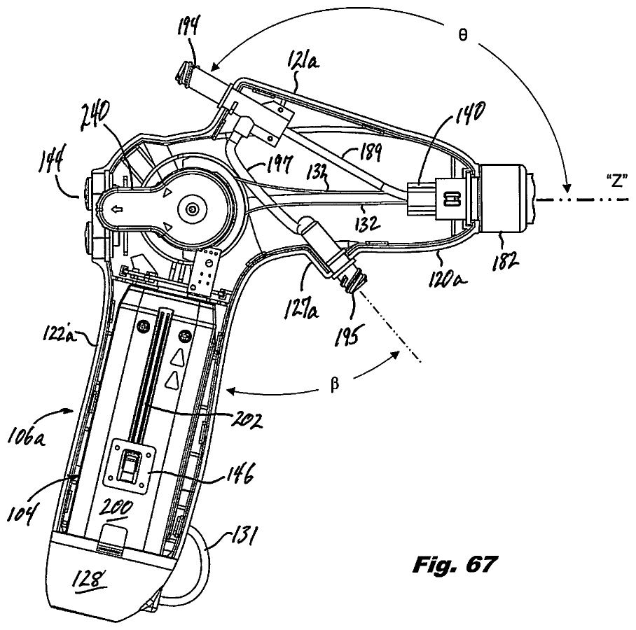

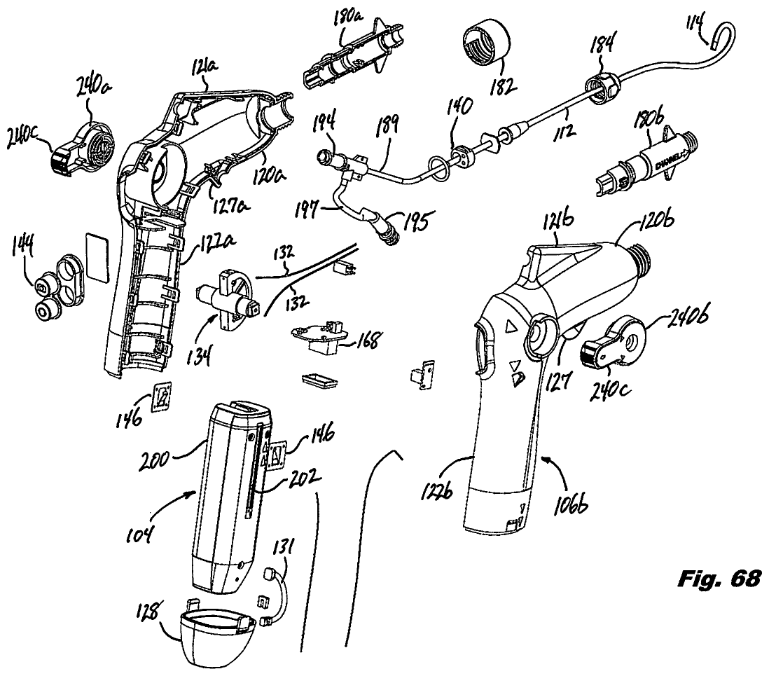

Referring now to FIGS. 63-68, another exemplary embodiment of a ureteroscope 100 according to the present disclosure is shown. In this exemplary embodiment, many of the components are designated by reference numerals discussed above and are the same as or similar to such like components and serve the same or similar functions as such like components. However, in this exemplary embodiment, the body 120 of the housing assembly 106 includes a second port extension 127 defined by a first extension portion 127a of the first housing portion 200a and a second extension portion 127b of the second housing portion 200b that when joined form the port extension 127. The second port extension 127 includes one or more luer ports 195 extending from a bottom side of the housing 200, as shown in FIG. 64. The one or more luer ports 195 of the second port extension 127 may be in communication, e.g., fluid communication, with the one or more luer ports 194 of the port extension 121 using, for example, a semi-rigid tube 197, shown in FIG. 67. In another exemplary embodiment, the one or more luer ports 195 of the second port extension 127 may be in communication, e.g., fluid communication, with the semi-rigid tube 189 connecting the one or more luer ports 194 to the channel 115 of the cannula 112 via the semi-rigid tube 197. In this exemplary embodiment, the one or more luer ports 194 remain fixed relative to the port extension 121 of the housing assembly 106, and the one or more luer ports 195 remain fixed relative to the port extension 127 of the housing assembly 106.

As seen in FIG. 63, this example of a ureteroscope 100 includes an optional switch 144a, which can be used in this or any other embodiment of endoscope or ureteroscope 100 to control additional functions such as commanding changes in imaging parameters such as image brightness by controlling the power to the LEDs in the imaging module 114 or by controlling image processing parameters, or storing freezeframes of a video being displayed, or some other operation of the imaging module or of motor 152 or of processor 1000 and display 1002.

FIG. 63 illustrates two sight members 192, one pointing up and another pointing down in the orientation shown in FIG. 63. While this is one example, it is preferable in at least some embodiments to eliminate the sight member 192 that points down to thereby eliminate a possible ambiguity, especially if handle 122 is rotated relative to the patient and room through a large angle. Accordingly, in preferred embodiments only the sight member 192 that points up in FIG. 63 is used. The example of FIG. 30 illustrates the use of only a single sight member 192.

For similar reasons, the arrow 129b that points down in the orientation seen in FIG. 63 can be eliminated in this and other examples of the endoscope or ureteroscope 100. In each case, the single sight member 192 and single arrow 129a are preferably colored black.

As seen most clearly in FIG. 66, hollow body or hub 180 has structural notation “CHANNEL” and an arrow pointing distally. This notation is on only one side of body 180, to inform the clinician that the sensor 114a is on the same side as the notation CHANNEL on hub 180 and the channel 115 is at the side of body or hub 180 that is not seen in FIG. 63. The notation CHANNEL and the arrow next to it can be formed such that they also provide a tactile indication so that a clinician can feel them with a finger to confirm the relative position of the sensor and channel. For example, the letters and arrow can be raised or indented. This applies to all examples of the endoscope of ureteroscope described in this patent specification.

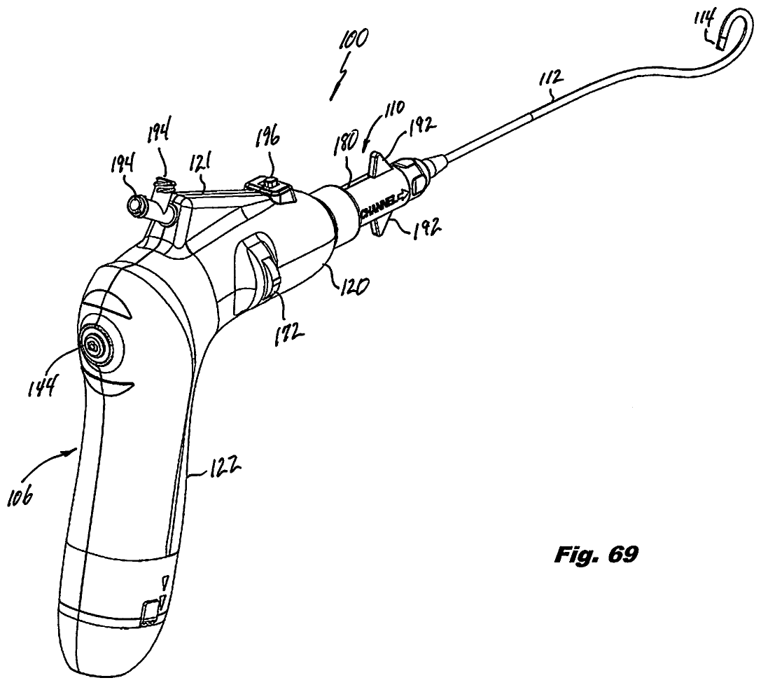

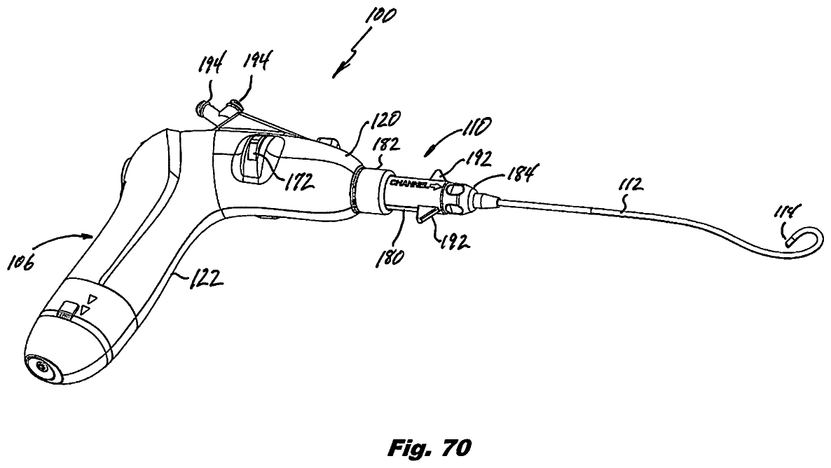

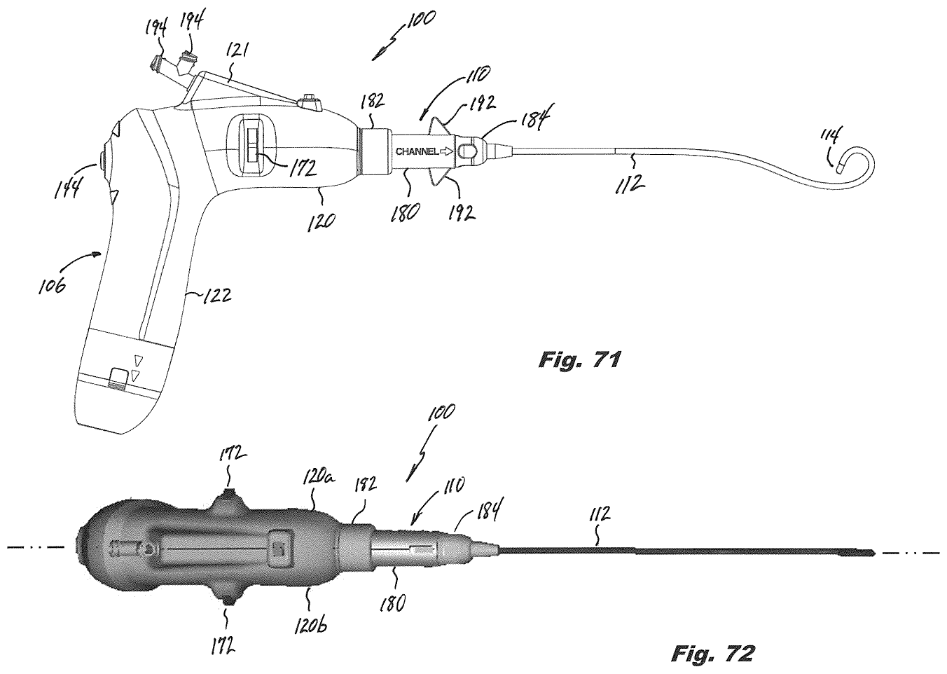

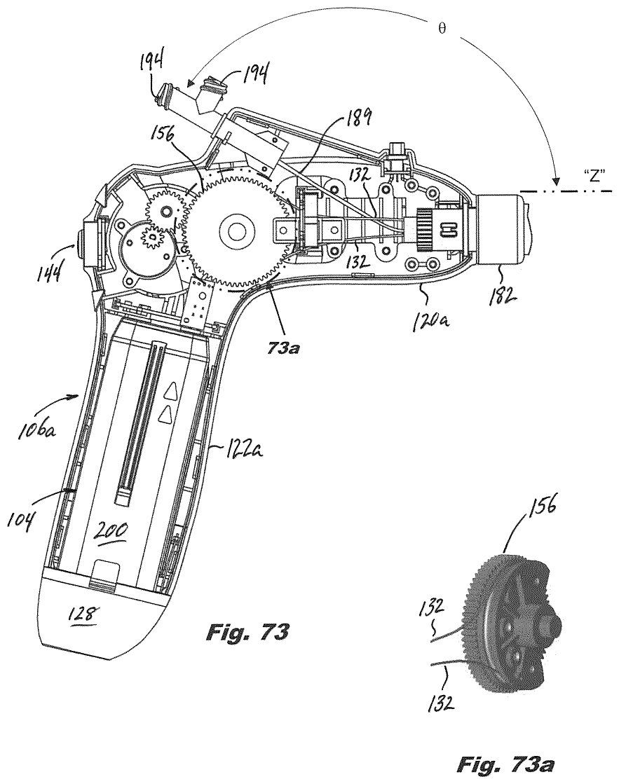

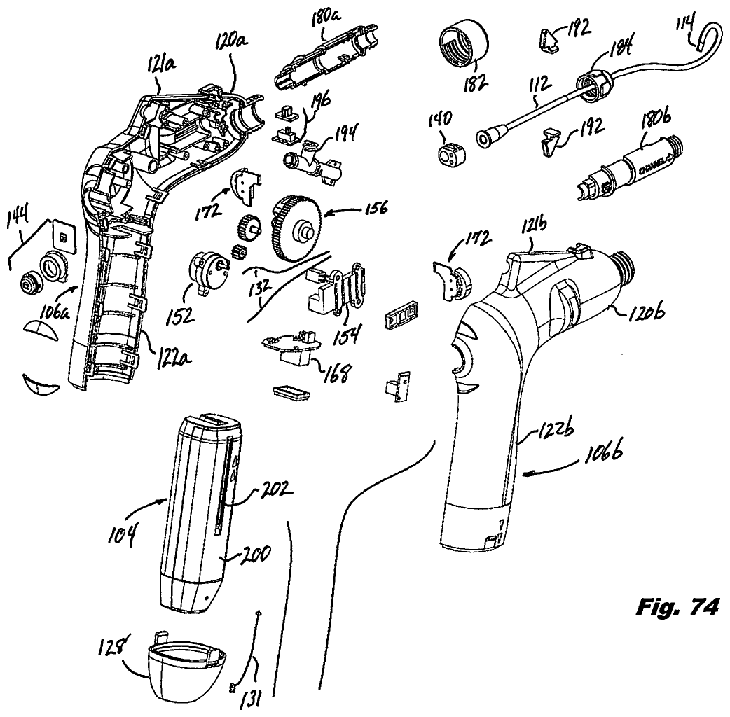

FIGS. 69-74 illustrate another exemplary embodiment of a ureteroscope 100 according to the present disclosure. This example is otherwise like the example illustrated in FIGS. 27-32 but uses a different way to control the deflection of angulation of the tip of cannula 112 and uses manual rotation of the cannula relative to the handle. In the example of FIGS. 69-74, a switch 172 that protrudes radially from each side of body 120 controls the deflection of the tip of cannula 112. Switch 172 can be a rocker switch or a rotary switch or any other suitable switch that enables control over deflection in both angular directions by being manually moved up or down or pressed at a top or bottom end. Switch 172 controls motor 152 in manner similar to the control over deflection by lever 138 in FIG. 27, except that gearing arrangement 156 is somewhat different in FIG. 74. Providing a mirror image switch 172 on the side not seen in FIG. 69 enables ambidextrous operation as the clinician can hold handle 122 in either hand. Rotation of cannula 112 relative to handle 122 is manual in this example. The clinician can grasp body 180 and manually rotate it relative to handle 122 while also holding handle 122 to thereby rotate cannula 112 relative to handle 122.

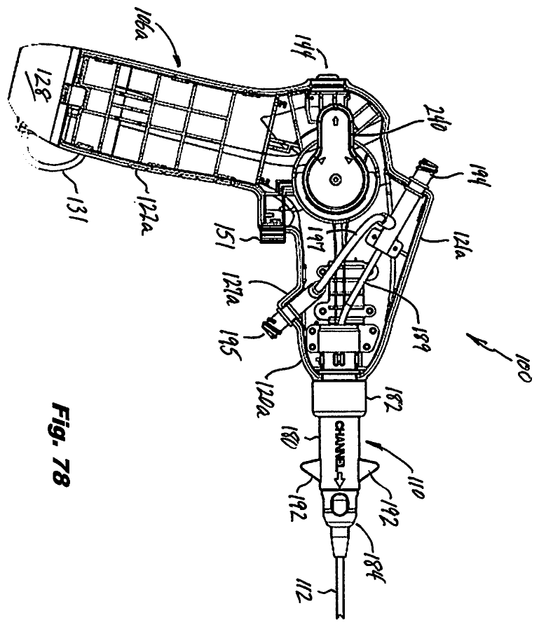

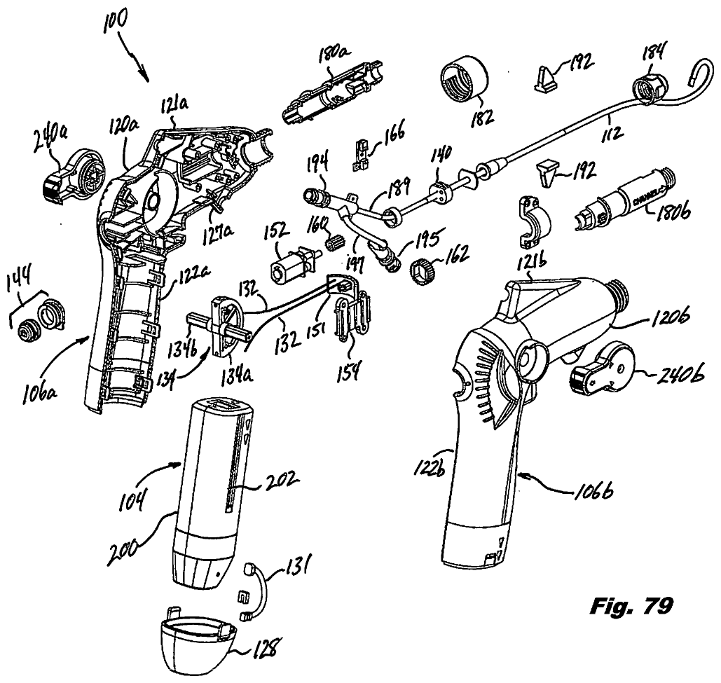

FIGS. 75-79 illustrate another exemplary embodiment of a ureteroscope 100 according to the present disclosure. This example is otherwise like the example illustrated in FIGS. 57-62 but differs in how it implements cannula tip deflection relative to the cannula axis of the cannula and how it implements cannula rotation relative to the handle. In the example of FIGS. 75-79, control over cannula tip deflection if through pivoting levers 240a and 240b. Mirror image levers are provided to make operation ambidextrous so the clinician can hold handle 122 in either hand to operate a lever 240 with a thumb. The levers operatively connect to motor 152 (FIG. 79) to cause the motor to deflect the cannula tip up when the clinician pivots either lever 240 up from a midpoint in the orientation of FIG. 75, or to deflect the cannula tip down when the clinician pivots the lever 240 down from the midpoint. Deflection motion stops when the lever 240 stops pivoting, and the deflection angle remains the same while the lever pivot position stays the same. The levers 240 rotate cable wheel 134 that in turn controls motor 152 to drive cannula tip deflection through gear arrangement 156.

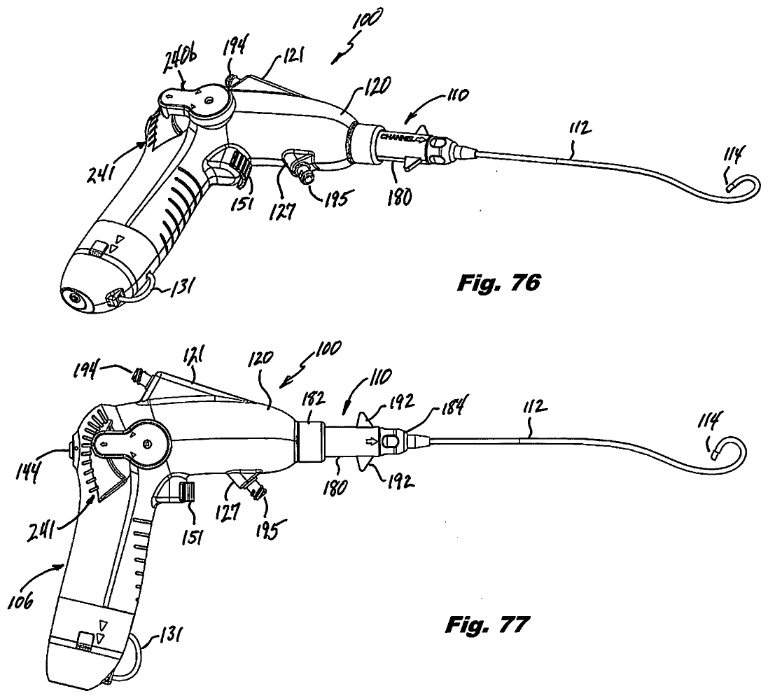

In this example, control over rotation of cannula 112 relative to handle 122 is through a slider 151 (FIG. 76) that slides to the left or to the right as seen in the distal direction from handle 122. When slider 151 is in a central position relative to handle 122, the cannula is as seen in FIGS. 75-77 and as seen in phantom lines in FIG. 39, i.e., the cannula is essentially straight. When the clinician starts moving slider 151 to the left or to the right from a central position, such as by using either forefinger, the cannula rotates in one direction or the opposite direction relative to handle 122. Slider 151 operatively connects to hub 180 and thus to cannula 112 to rotate the hub and c cannula as a unit relative to handle 122 about the cannula's long axis. For example, the cannula rotates from the deflection plane seen in FIG. 39 toward and then to one or the other of the deflection planes seen in FIGS. 40 and 41. Rotation stops when motion of slider 151 stops so that a rotation position is maintained while slider 151 is held in one position. While the positions of the ports for surgical instruments and fluid differ between the examples of FIGS. 39-41 and FIGS. 75-79, the motion of cannula 112 is the same.

An additional feature of the example of FIGS. 75-79 are structural marks 241 on each side of body 120, just proximal to the paths of the proximal ends of levers 240. Marks 240 enable visual and, if desired tactile, indication of the degree of pivoting of levers 240. Marks 241 can be raised ridges or indentations into body 120, configured to help the clinician hold levers 240 at a desired position by keeping the clinician's thumb from slipping over marks 241.

Notably, tube 189 (FIG. 78) enables rotation of cannula 112 relative to handle 122 without interference with smooth entry of surgical implements into channel 115 or movements of such implements in the channel.

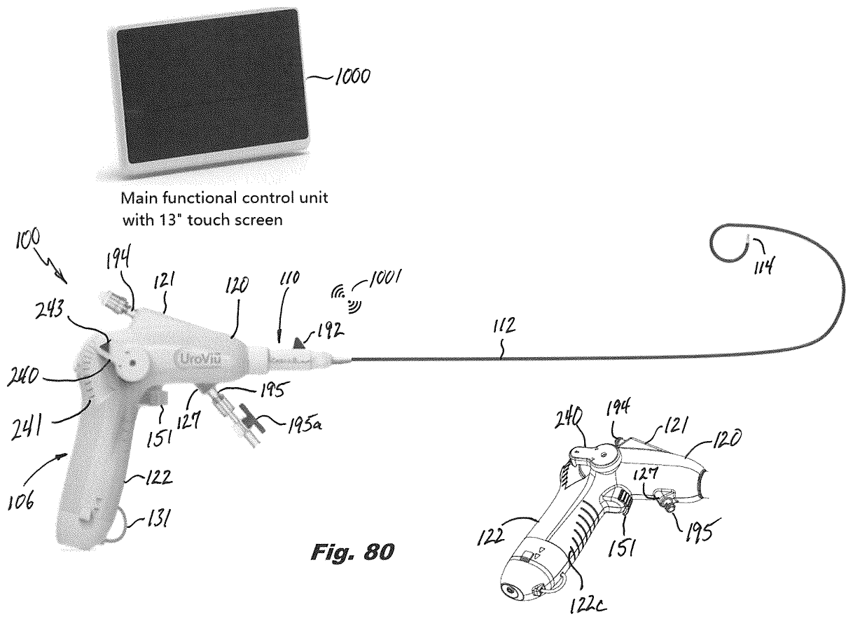

FIG. 80 illustrates a currently preferred example of endoscope of ureteroscope 100 that is otherwise like FIG. 75 but is to scale of a selected product and additionally shows an arrow mark 243 on lever 240. Mark 243 points up in the orientation seen in FIG. 80 and indicates the direction in which the cannula tip is deflected when lever 240 moves up, in the direction of the arrow. In FIG. 80, lever 240 has moved up and the tip of cannula 112 has deflected up from the long axis of the cannula. In addition. FIG. 80 shows only a single sight member 192, colored black, rather than the less preferred option of using two sight members as in FIG. 75 for example. FIG. 80 further includes a perspective view of handle 122 and body 120, showing optional ridges of indentations 122c at the distal side of handle 120, to help prevent the handle slipping in the clinician's hand. Still in addition, FIG. 80 illustrates external processing/display unit 1000, to which endoscope/ureteroscope 100 can connect wirelessly, for example through a WiFi link 1001, or through cable such as illustrated in FIG. 14.

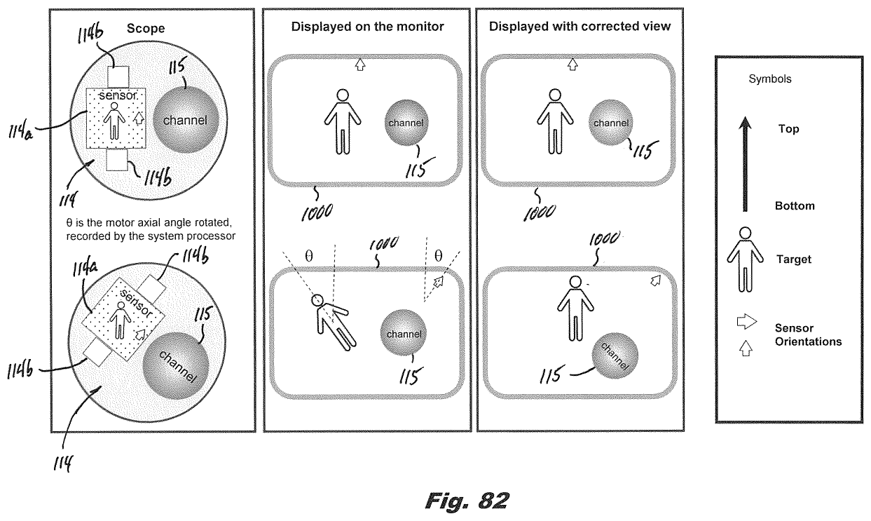

FIGS. 81 and 82 illustrate images that can be seen on a display 1002 in processor unit 1000 for several different positions of cannula 112 relative to a patient.

FIG. 81 shows in the left panel two possible orientations of camera (sensor) 114a and channel 115 (such as a working channel) relative to the patient and the room. In the upper image channel 115 is to the right of the sensor 114a and in the lower image the cannula has been rotated 90 degrees so the channel is below the sensor. The arrows in the sensor icons indicate the direction of view of the sensor. The middle panel shows an upper image with a patient icon and shows channel 115 and an arrow pointing up to indicate the direction of view of the sensor relative to the channel. The lower image shows an arrow indicating the rotated position of the direction of view of the sensor and still shows the channel 115 but also has rotated the patient icon by carrying out a simple image rotation. This view may not be intuitively understood.

To make the displayed view more informative and intuitive, ureteroscope 100 and/or unit 1000 are configured with electronics to generate and display the images seen in the third panel from the left in FIG. 81, which panel is labeled “Displayed with corrected view.” In this panel, the upper image is the same as in the panel on the immediate left but the lower image correctly indicates that the channel 115 is below the sensor 114a and that the sensor's viewpoint is to the right. The rightmost panel shows the symbols used in the other panels.,

FIGS. 82 is otherwise like FIG. 81 but shows a rotation of cannula 112 by 45 degrees (angle Theta). In this case, the lower image in the second panel from the left has been corrected to the lower image in the third panel. The meaning of the arrows and of the position of the channel 115 relative to sensor 114a is the same as in the FIG. 81.

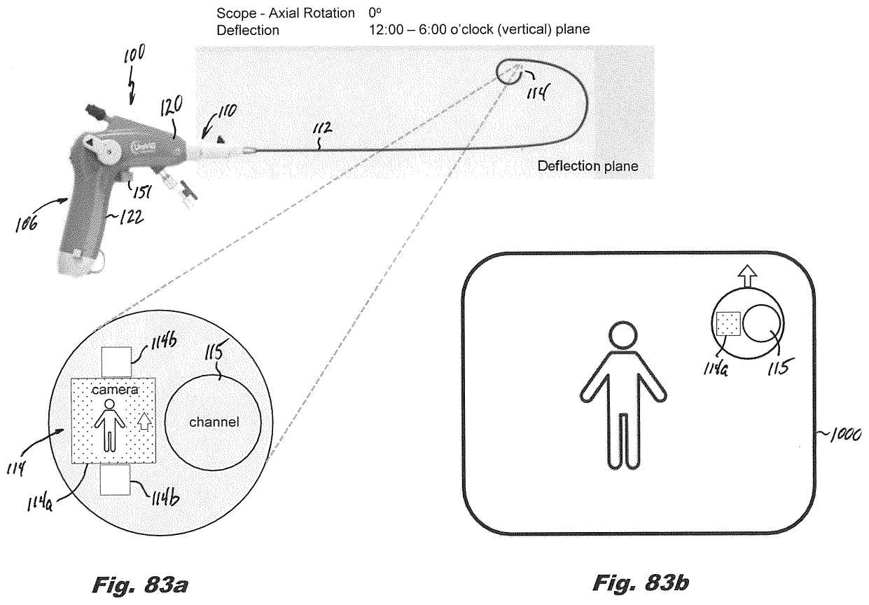

FIGS. 83a-85b illustrate images that can be seen on the screen 1002 of unit 1000 for various positions of the deflection plane of cannula 112 relative to handle 122. FIG. 83a shows a deflection plane that is vertical relative to a coordinate system such as of a room and channel 115 is to the right of sensor 114a. FIG. 83b shows the corrected image displayed on the screen of unit 1000, with the patient icon and an icon of the sensor and channel and an arrow indicative of positions and direction of view. FIGS. 84a and 84b are similar but here the deflection plane is horizontal relative to the room and the tip of cannula 112 is deflected to the left. The displayed image seen in FIG. 74b correctly shows the positional relationships. The arrow in this case points to the left. FIGS. 85a and 85b are otherwise like FIGS. 84a and 84b but in this case the tip of cannula 112 is deflected to the right, so that the arrow in FIG. 85b points to the right.

FIGS. 86-89 illustrate an example of steps in using the endoscope/ureteroscope 100 and of images that can be shown on the display of unit 1000. In step 1, the display enables the clinician to select the kidney to be worked on by tapping on the icon of the right or left kidney. If the ureteroscope 100 is in the illustrated default position relative to the room, i.e., with the deflection plane vertical, the display shows a circle with a dash line indicating the deflection plane orientation relative to the room and that the channel is to the right of the sensor. In step 2, the clinician has selected the right kidney and the display highlights the icon for the right kidney—orange in this example. In step 3, the clinician inserts the cannula 112 into the patient. There are several known ways to get the cannula tip to and into the kidney. One is to use a guidewire under fluoroscopic or other imaging to reach the kidney and then to thread the cannula over the guidewire. In step 3, the clinician has guided the cannula tip in the right kidney and has used rotation control 150 to rotate the cannula deflection plane to a roughly horizontal position, with the direction of view of the sensor toward the interior of the kidney. Unit 1000 displays the highlighted right kidney, with a symbol indicating that the tip of the cannula is into the kidney and displays in the circle below a horizontal dash line to indicate the orientation of the deflection plane and also displays in the circle the position of the channel 115 above the dashed line to show that the channel is above the sensor. In step 4, the clinician has turned on sensor 114a so the display shows an interior area of the kidney and has rotated the cannula 112 relative to the handle 122 so the deflection plane is angled to the horizontal, by sliding slider 151 or rotating handle 122 relative to the patient and room.

In or after step 4, the clinician can pass through channel 115 a surgical implement such as a laser fiber optic cable or bundle to act on kidney stones, Tube 189 (FIG. 78) is configured to enable such passage and manipulation of a surgical implement in channel 115 despite rotation of cannula 115 relative to handle 122, without twisting or interfering with the surgical implement. In a preferred example, tube 189 is flexible and made of a material such as Teflon, is at least 200 mm long, is continuous, and is longer than the distance along a straight from where its proximal and distal ends attach. Tube 189 hangs free, without any attachments or seams between its proximal and distal ends, and its length is sufficient to allow for rotation of the cannula relative to the handle by at least 180 degrees in each direction without stretching or pinching tube 189.

Notably, rotation of the deflection plane (of the cannula) can be done in three ways. One is by motorized deflection using control system 130, motor 152, and slider 151 (FIG. 76). Another is to manually rotate hollow body or hub 180 relative to handle 122. Yet another is to rotate handle 122 about the cannula axis relative to the patient and the room. According to some examples, motorized rotation can be used to rotate the deflection plane through greater angles, such as angles of 22 degrees or more, and to use manual rotation to finely tune the orientation of the deflection plane, for example through angles of less than 22 degrees.

Axial rotation of the cannula 112 relative to the handle 122 to change the deflection plane of the cannula and independent deflection of the cannula tip relative to the cannula's long axis enable the sensor 114 and the distal opening of channel 115 to traverse the entire target space in a kidney. Using control over rotation and deflection as described above, for example motorized rotation and deflection using motor 152, and a pistol-grip handle 122, have been found to be more ergonomic and lower-cost that known ureteroscopes.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered as illustrative and not restrictive, and the body of work described herein is not to be limited to the details given herein, which may be modified within the scope and equivalents of the appended claims.

What is claimed is:

1. An ergonomic ureteroscope with power-assisted, controlled cannula rotation relative to a handle and concurrently operable cannula tip deflection control, comprising:
a housing, a pistol-grip handle extending down from the housing, a cannula extending distally from the housing along a cannula axis, a channel through the cannula, and an imaging module at a distal tip of the cannula, wherein the cannula is sufficiently long to reach into an adult patient's kidneys through the patient's urethra and ureter passages;
an upper port that is at a proximal upper portion of the housing, is inclined toward the proximal direction, and is configured to communicate with the channel through the cannula, and a lower port that at a lower portion of the housing, is inclined toward the distal direction, and also is configured to communicate with the channel through the cannula;
a motorized deflection drive system configured to deflect the distal tip of the cannula relative to the cannula axis in opposed angular directions in a cannula deflection plane, said assembly comprising a motor located in the housing and operatively coupled to the cannula to drive deflection of the cannula and a thumb-operated pivoting lever on each side of the housing for ambidextrous control over the motor to initiate the deflection and to set the deflection angle by pivoting the lever; and
a rotation drive system configured to rotate the cannula and thus the cannula deflection plane relative to the housing and comprising a finger operated slider that is at a front side of a proximal portion of the handle and is operatively coupled to the cannula to rotate the cannula relative to handle through an angle corresponding to sliding motion of the slider relative to the handle;
wherein the deflection drive system and the rotation drive system are configured to enable concurrent operation to rotate the cannula and deflection plane relative to the handle and to deflect the cannula tip relative to the cannula axis.

2. The ureteroscope of claim 1, further including an image processor and display operatively coupled with the deflection drive system and with the rotation drive system and configured to display an image that shows a current relative position of the imaging module and the channel and the current direction of view of the imaging module as the cannula rotates relative to the handle and the cannula tip deflects relative to the cannula axis.

3. The ureteroscope of claim 2, in which the image processor and display are further configured to show an icon of a patient such that the displayed current relative position and direction of view also indicate how the patient's is positioned relative to the imaging module, the channel, and the direction of view.

4. The ureteroscope of claim 2, in which the image processor and display are further configured to display icons of left and right kidneys in a first step of a medical procedure and in response to a tap on a selected kidney icon to highlight the kidney icon and to show entry of the cannula tip in the kidney.

5. The ureteroscope of claim 4, in which the image processor and display are further configured to show an image of the kidney interior taken with the imaging module and to respond to rotation of the deflection plane after the cannula tip has entered the kidney to update the displayed current relative position and direction of view.

6. The ureteroscope of claim 1, further including a hub between the housing and the cannula, which hub is configured to rotate with the cannula relative to the handle, and a structural indicium on a side of the hub indicative of the relative position of the imaging module and the channel.

7. The ureteroscope of claim 6, further including a sight member extending up from the hub when the hub is at an initial rotation position relative to the handle and rotating with the hub as the cannula rotates relative to the handle.

8. The ureteroscope of claim 1, including a port extension rising up from the housing supporting the upper port at a position spaced up from the housing to facilitate insertion of a surgical instrument through the upper port without interfering with the pivoting levers.

9. The ureteroscope of claim 1, further including structural marks on the housing that are adjacent a moving end of the pivoting levers and are configured to facilitate holding the pivoting levers in position and avoid slippage of a finger operating the pivoting levers.

10. A ureteroscope comprising:
a single-use portion including:
a housing assembly having a body and a pistol-grip handle that extends away from the body and has a proximal part that is integral with the housing and an open distal part;
a hub assembly extending distally along a cannula axis from the body, the hub assembly having a body and two proximal ports that extend in different directions transverse to the cannula axis such that at least one of the ports is in a line of sight along the cannula axis from a viewpoint a selected distance proximal from the housing assembly;
a cannula that extends distally from the hub assembly along the cannula axis and has an imaging module at a distal part, wherein the hub assembly and the cannula are mounted for rotation about the cannula axis relative to the housing assembly body and the cannula is sufficiently long and flexible to reach a kidney of an adult patient when inserted through a urethra, bladder and ureter;
an internal channel that has a proximal part at said proximal ports and extends therefrom to a distal port at a distal part of the cannula and is configured to provide a path for one or more surgical implements through one of both of said proximal ports to and distally out of the distal part;
a rotation drive system located at the housing assembly and operatively coupled with the hub assembly to rotate the hub assembly and the cannula about the cannula axis relative to the housing assembly, the rotation drive system including a manually operated control configured to move relative to the handle to provide a visual and tactile indication of a rotated position of the cannula relative to the handle; and
a deflection control system located at the housing assembly and interactive with the distal part of the cannula, such that when the deflection control system is activated the distal part of the cannula deflects in a positive direction between a first position and a second position along and in a negative direction between the first position and a third position along a deflection plane;
wherein the positions of the proximal ports relative to the handle provide an immediate visual and tactile indication of the deflection plane in which the distal part of the cannula is located relative to the orientation of the handle;
a reusable portion including:
a housing having a proximal part and a distal part and configured for insertion in the open distal part of the handle;
at least one electrical contact at the proximal part of the housing and accessible from an exterior of the reusable portion housing, the at least one electrical contact is configured to mate with one or more electrical contacts within the handle when the proximal part of the housing is inserted in the handle; and
a cap configured to releasably mate with the open distal part of the handle so that when the reusable portion is inserted in the open distal part of the handle the cap covers the open distal part of the handle and encloses the distal part of the reusable portion housing.

11. An ureteroscope comprising:
a single-use portion including:
a housing assembly having a body and a handle extending away from the housing body, the handle has a proximal part that is integral with the housing and an open distal part;
a hub assembly extending distally along a cannula axis from the body, the hub assembly having a body and at least one port extending from the body at a predefined angle relative to the hub assembly body;
a cannula that extends distally from the hub assembly along the cannula axis and has an imaging module at a distal part, wherein the hub assembly and the cannula are mounted for rotation about the cannula axis relative to the housing assembly body, the cannula having at least one channel extending from a proximal part of the cannula to the distal part of the cannula;
a rotation drive system located within the housing assembly and operatively coupled with the hub assembly to rotate the hub assembly and the cannula about the cannula axis; and
a deflection control system located within the housing assembly and interactive with the distal part of the cannula, such that when the deflection control system is activated the distal part of the cannula can deflect in a positive direction between a first position and a second position along a deflection plane, or the distal part of the cannula can deflect in a negative direction between the first position and a third position along the deflection plane;
a reusable portion including:
a housing having a proximal part and a distal part and configured for insertion in the open distal part of the handle;
at least one electrical contact at the proximal part of the housing and accessible from an exterior of the reusable portion housing, the at least one electrical contact is configured to mate with one or more electrical contacts within the handle when the proximal part of the housing is inserted in the handle; and
a cap configured to releasably mate with the open distal part of the handle so that when the reusable portion is inserted in the open distal part of the handle the cap covers the open distal part of the handle and encloses the distal part of the reusable portion housing.

12. The ureteroscope of claim 11, wherein the rotation drive system comprises a powered rotation drive system to rotate the hub assembly and the cannula about the cannula axis.

13. The ureteroscope of claim 12, wherein the powered rotation drive system comprises at least one selectively activated motor to rotate the hub assembly and the cannula about the cannula axis.

14. The ureteroscope of claim 12, wherein the rotation drive system comprises a manual rotation drive system to rotate the hub assembly and the cannula about the cannula axis.

15. The ureteroscope of claim 14, wherein the manual rotation drive system comprises at least one thumbwheel to rotate the hub assembly and the cannula about the cannula axis.

16. The ureteroscope of claim 11, wherein the deflection control system comprises a manually operated deflection control system interactive with the distal part of the cannula.

17. The ureteroscope of claim 11, wherein the manually operated deflection control system comprises a lever positioned on an exterior of the housing assembly and at least one push-pull cable operatively connected to the lever and the cannula such that motion of the lever in a first direction causes the distal part of the cannula to deflect in a positive direction between the first position and the second position, and motion of the lever in a second direction causes the distal part of the cannula to deflect in a negative direction between the first position and the third position.

18. The ureteroscope of claim 11, wherein the manually operated deflection control system comprises at least one deflection wheel lever positioned on an exterior of the housing assembly and at least one push-pull cable operatively connected to the deflection wheel lever and the cannula such that motion of the deflection wheel lever in a first direction causes the distal part of the cannula to deflect in a positive direction between the first position and the second position, and motion of the deflection wheel lever in a second direction causes the distal part of the cannula to deflect in a negative direction between the first position and the third position.

19. The ureteroscope of claim 11, wherein the reusable portion includes a battery and control and processing electronics configured to control the imaging module to take images in a field of view and to receive image data from the imaging module.

20. The ureteroscope of claim 11, wherein the reusable portion includes a facility to convey image data from the endoscope to an external processing/display unit.

21. The ureteroscope of claim 20, wherein the facility is configured to convey image data by wireless transmission using a Point-to-Point Wi-Fi protocol.

22. The ureteroscope of claim 21, in which said facility in the reusable portion is configured to convert received image data into display images and convey display images to an external unit for display.

23. The ureteroscope of claim 11, further comprising a manual switch at the distal part of the reusable portion, wherein at least the portion of the cap that is over the switch is sufficiently flexible for manual operation of the switch through the cap.
